SEQUENCE LISTING (559 sequences)

SEQ ID NO: 1 - protein, 683 aa, Homo sapiens
DYKDDDDKGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNFHHHHHH

SEQ ID NO: 2 - protein, 656 aa, Macaca fascicularis
DYKDDDDKAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEFHHHHHH

SEQ ID NO: 3 - protein, 656 aa, Callithrix jacchus
DYKDDDDKVPQLYWDDLKKMLSEKLDTTDFTSTIKLLNENSYVPREAGSQKDENLAWYIENQFRECKLSKVWRDEHFVKIQVKDSAQNSVTITGTNSEFVYLVENPGGYVAYSQNATVTGKLVHANFGTEKDFEDLDSPVNGSLVIVRAGKITFAEKVANAESANAIGVLIYMDKTKFPIVDADVSFFGHAHLGTGDPYTPGFPSFNHTQFPPSRSSGLPNIPVQTISRAAAERLFENMEGDCPSDWKTDSSCRMVTSQNKSVKLSVSNVMKEIKILNIFGVIKGFVEPDRYVVVGAQRDSWGPGAAKSSVGTALLLELAQTFSDMVLKDGFQPSRSIVFASWSAGDFGSVGATEWLEGYLSSLHLKAFTFINLDKAVLGTSNFRVSASPLLYALIEKTMQEVKHPVTGLSLYQDSNWASKVEKLTFDNAAFPFLAYSGIPAVSFCFCEDTDYPYLGTTMDTYKELIEKIPQLNKVARAAAEVAGQFMIKLTHDVELNLDYERYNSQLLSFLRNLNQYRADIREMGLSLQWLYSARGDFFRATSRLTTDFKNSEKTDRFVMKELNDRVMKVEYYFLSPYVSPRESPFRHIFWGSGSHTLSALLEHLKLRKTNNRAFNETLFRNQLALATWTIQGAANALSGDIWDIDNEFHHHHHH

SEQ ID NO: 4 - protein, 655 aa, Rattus rattus
DYKDDDDKSSRLFWADLKTLLSEKLNSIEFTDIIKQLSQNTYTPREAGSQKDENLAYYIENLFHDFKFSKVWRDEHYVKIQVKNSVSQNLVTINSGSNIDPVEAPEGYVAFSKAGEVTGKLVHANFGTKKDFEELNYSVNGSLVIVRAGKITFAEKVANAQSFNAIGVLIYMDRNTFPVVEADLQFFGHAHLGTGDPYTPGFPSFNHTQFPPSQSSGLPSIPVQTISRAAAEKLFKNMEGNCPPSWNIDSSCKLELSQNQNVKLTVNNVLKETRILNIFGVIKGYEEPDRYIVVGAQRDAWGPGVAKSSVGTGLLLKLAQVFSDMISKDGFRPSRSIIFASWTAGDYGAVGATEWLEGYLSSLHLKAFTYINLDKVVLGTSNFKVSASPLLYTLMGKIMQDVKHPIDGKYLYRDSNWISKIEELSLDNAAFPFLAYSGIPAVSFCFCEDEDYPYLGTKLDTYEILIQKVPQLNQMVRTAAEVAGQFIIKLTHDIELTLDYEMYNSKLLSFMKDLNQFKADIKDMGLSLQWLYSARGDYFRATSRLTTDFHNAEKTNRFVMREINDRIMKVEYHFLSPYVSPRESPFRHIFWGSGSHTLSALVENLRLRQKNITAFNETLFRNQLALATWTIQGVANALSGDIWNIDNEFHHHHHH

SEQ ID NO: 5 - protein, 657 aa, Mus musculus
DYKDDDDKSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEFHHHHHH

SEQ ID NO: 6 - protein, 129 aa, synthetic construct
  REGION 1..129: note = TfR1 (VHH)
QVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 7 - protein, 8 aa, synthetic construct
  REGION 1..8: note = TfR1 HCDR1
GLTFSNYA

SEQ ID NO: 8 - protein, 8 aa, synthetic construct
  REGION 1..8: note = TfR1 HCDR2
IGGSGGTW

SEQ ID NO: 9 - protein, 22 aa, synthetic construct
  REGION 1..22: note = TfR1 HCDR3
AADQRAGSYSSGWYTRSSDSLY

SEQ ID NO: 10 - protein, 584 aa, synthetic construct
  REGION 1..584: note = BBBB434 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 11 - protein, 218 aa, synthetic construct
  REGION 1..218: note = BBBB434 LC
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 449 aa, synthetic construct
  REGION 1..449: note = BBBB434 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 13 - protein, 584 aa, synthetic construct
  REGION 1..584: note = BBBB978 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 14 - protein, 215 aa, synthetic construct
  REGION 1..215: note = BBBB978 LC
EIVMTQSPATLSVSPGERATLSCRASQSVSSNLAWYQQKPGQAPRLLIYGASTRATGIPARFSGSGSGTEFTLTISSLQSEDFAVYYCQQLILWPPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, synthetic construct
  REGION 1..449: note = BBBB978 HC2
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 16 - protein, 582 aa, synthetic construct
  REGION 1..582: note = BBBB1009 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 17 - protein, 214 aa, synthetic construct
  REGION 1..214: note = BBBB1009 LC
DIQMTQSPSSLSASVGDRVTITCKASQDINRYLNWFQQKPGKAPKTLIYRANRLLDGVPSRFSGSGSGTDFTLTISSLQPEDFATYYCQQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 447 aa, synthetic construct
  REGION 1..447: note = BBBB1009 HC2
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 19 - protein, 582 aa, synthetic construct
  REGION 1..582: note = BBBB1011 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 20 - protein, 214 aa, synthetic construct; identical to SEQ ID NO: 17
  REGION 1..214: note = BBBB1011 LC

SEQ ID NO: 21 - protein, 420 aa, synthetic construct
  REGION 1..420: note = BBBB1011 HC2
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQ

SEQ ID NO: 22 - protein, 580 aa, synthetic construct
  REGION 1..580: note = BBBB1073 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 23 - protein, 219 aa, synthetic construct
  REGION 1..219: note = BBBB1073 LC
DVVMTQSPLSLPVTLGQPASISCKSSQSLLDSRAKTYLTWLQQRPGQSPRRLIYLVSKLDSGVPDRFSGSGSGTDFTLKISRVEAEDVGVYYCWQGTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 445 aa, synthetic construct
  REGION 1..445: note = BBBB1073 HC2
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 25 - protein, 581 aa, synthetic construct
  REGION 1..581: note = BBBB1215 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLVESGGGLVQTGGSLRLSCAASGLTFSNYAMGWFRQAPGKKREFVAHIGGSGGTWRYADSVKGRFTISRDNAKNMVYLQMNSLNTEDTAIYYCAADQRAGSYSSGWYTRSSDSLYWGQGTQVTVSS

SEQ ID NO: 26 - protein, 218 aa, synthetic construct; identical to SEQ ID NO: 11
  REGION 1..218: note = BBBB1215 LC

SEQ ID NO: 27 - protein, 446 aa, synthetic construct
  REGION 1..446: note = BBBB1215 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVYPPSQEEMTKNQVSLTCLVKGFYPSDIAVEWESNGQPENNYKTTPPVLDSDGSFALVSRLTVDKSRWQEGNVFSCSVMHEALHNHYTQKSLSLSLG

SEQ ID NO: 28 - protein, 248 aa, synthetic construct
  REGION 1..248: note = TfR2 (ScFV)
EVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 29 - protein, 8 aa, synthetic construct
  REGION 1..8: note = TfR2 HCDR1
GFTFRNAW

SEQ ID NO: 30 - protein, 9 aa, synthetic construct
  REGION 1..9: note = TfR2 HCDR2
IKRKIDGGT

SEQ ID NO: 31 - protein, 15 aa, synthetic construct
  REGION 1..15: note = TfR2 HCDR3
TTDPSRIPVAGAFDY

SEQ ID NO: 32 - protein, 7 aa, synthetic construct
  REGION 1..7: note = TfR2 LCDR1
QSVSSTY

SEQ ID NO: 33 - protein, 7 aa, synthetic construct
  REGION 1..7: note = TfR2 LCDR2
GASSRAT

SEQ ID NO: 34 - protein, 9 aa, synthetic construct
  REGION 1..9: note = TfR2 LCDR3
QQYGSSPYT

SEQ ID NO: 35 - protein, 703 aa, synthetic construct
  REGION 1..703: note = BBBB501 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 36 - protein, 218 aa, synthetic construct; identical to SEQ ID NO: 11
  REGION 1..218: note = BBBB501 LC

SEQ ID NO: 37 - protein, 449 aa, synthetic construct; identical to SEQ ID NO: 12
  REGION 1..449: note = BBBB501 HC2

SEQ ID NO: 38 - protein, 703 aa, synthetic construct
  REGION 1..703: note = BBBB951 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSEVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 39 - protein, 218 aa, synthetic construct; identical to SEQ ID NO: 11
  REGION 1..218: note = BBBB951 LC

SEQ ID NO: 40 - protein, 449 aa, synthetic construct; identical to SEQ ID NO: 12
  REGION 1..449: note = BBBB951 HC2

SEQ ID NO: 41 - protein, 703 aa, synthetic construct
  REGION 1..703: note = BBBB979 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 42 - protein, 215 aa, synthetic construct; identical to SEQ ID NO: 14
  REGION 1..215: note = BBBB979 LC

SEQ ID NO: 43 - protein, 449 aa, synthetic construct; identical to SEQ ID NO: 15
  REGION 1..449: note = BBBB979 HC2

SEQ ID NO: 44 - protein, 701 aa, synthetic construct
  REGION 1..701: note = BBBB1020 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 45 - protein, 214 aa, synthetic construct; identical to SEQ ID NO: 17
  REGION 1..214: note = BBBB1020 LC

SEQ ID NO: 46 - protein, 447 aa, synthetic construct
  REGION 1..447: note = BBBB1020 HC2
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 47 - protein, 701 aa, synthetic construct
  REGION 1..701: note = BBBB1018 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 48 - protein, 214 aa, synthetic construct; identical to SEQ ID NO: 17
  REGION 1..214: note = BBBB1018 LC

SEQ ID NO: 49 - protein, 447 aa, synthetic construct; identical to SEQ ID NO: 18
  REGION 1..447: note = BBBB1018 HC2

SEQ ID NO: 50 - protein, 699 aa, synthetic construct
  REGION 1..699: note = BBBB1076 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 51 - protein, 219 aa, synthetic construct; identical to SEQ ID NO: 23
  REGION 1..219: note = BBBB1076 LC

SEQ ID NO: 52 - protein, 445 aa, synthetic construct; identical to SEQ ID NO: 24
  REGION 1..445: note = BBBB1076 HC2

SEQ ID NO: 53 - protein, 700 aa, synthetic construct
  REGION 1..700: note = BBBB1216 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSEVQLVESGGGLVKPGGSLRLSCAASGFTFRNAWMSWVRQAPGKGLEWVGRIKRKIDGGTTDYAAPVKGRFTISRDDSKNTLYLQMNSLKTEDTAVYYCTTDPSRIPVAGAFDYWGQGILVTVSSGTEGKSSGSGSESKSTEIVLTQSPGTLSLSPGERATLSCRASQSVSSTYLAWYQQKPGQAPRLLIYGASSRATGIPDRFSGSGSGTDFTLTISSLEPEDFAVYYCQQYGSSPYTFGQGTKLEIK

SEQ ID NO: 54 - protein, 218 aa, synthetic construct; identical to SEQ ID NO: 11
  REGION 1..218: note = BBBB1216 LC

SEQ ID NO: 55 - protein, 446 aa, synthetic construct; identical to SEQ ID NO: 27
  REGION 1..446: note = BBBB1216 HC2

SEQ ID NO: 56 - protein, 246 aa, synthetic construct
  REGION 1..246: note = TfR3 (scFv)
EVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 57 - protein, 11 aa, synthetic construct
  REGION 1..11: note = TfR3 HCDR1
GFTFSSYNMNW

SEQ ID NO: 58 - protein, 10 aa, synthetic construct
  REGION 1..10: note = TfR3 HCDR2
SSISSSSSYI

SEQ ID NO: 59 - protein, 13 aa, synthetic construct
  REGION 1..13: note = TfR3 HCDR3
AREGISAYDALNV

SEQ ID NO: 60 - protein, 9 aa, synthetic construct
  REGION 1..9: note = TfR3 LCDR1
SSSNIGNNY

SEQ ID NO: 61 - protein, 7 aa, synthetic construct
  REGION 1..7: note = TfR3 LCDR2
DNNKRPS

SEQ ID NO: 62 - protein, 11 aa, synthetic construct
  REGION 1..11: note = TfR3 LCDR3
GTWDSSLSAVV

SEQ ID NO: 63 - protein, 701 aa, synthetic construct
  REGION 1..701: note = BBBB509 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 64 - protein, 218 aa, synthetic construct; identical to SEQ ID NO: 11
  REGION 1..218: note = BBBB509 LC

SEQ ID NO: 65 - protein, 449 aa, synthetic construct; identical to SEQ ID NO: 12
  REGION 1..449: note = BBBB509 HC2

SEQ ID NO: 66 - protein, 701 aa, synthetic construct
  REGION 1..701: note = BBBB946 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 67 - protein, 218 aa, synthetic construct; identical to SEQ ID NO: 11
  REGION 1..218: note = BBBB946 LC

SEQ ID NO: 68 - protein, 449 aa, synthetic construct
  REGION 1..449: note = BBBB946 HC2
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 69 - protein, 701 aa, synthetic construct
  REGION 1..701: note = BBBB974 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 70 - protein, 215 aa, synthetic construct; identical to SEQ ID NO: 14
  REGION 1..215: note = BBBB974 LC

SEQ ID NO: 71 - protein, 449 aa, synthetic construct; identical to SEQ ID NO: 15
  REGION 1..449: note = BBBB974 HC2

SEQ ID NO: 72 - protein, 699 aa, synthetic construct
  REGION 1..699: note = BBBB1023 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 73 - protein, 214 aa, synthetic construct; identical to SEQ ID NO: 17
  REGION 1..214: note = BBBB1023 LC

SEQ ID NO: 74 - protein, 447 aa, synthetic construct; identical to SEQ ID NO: 46
  REGION 1..447: note = BBBB1023 HC2

SEQ ID NO: 75 - protein, 699 aa, synthetic construct
  REGION 1..699: note = BBBB1021 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 76 - protein, 214 aa, synthetic construct; identical to SEQ ID NO: 17
  REGION 1..214: note = BBBB1021 LC

SEQ ID NO: 77 - protein, 447 aa, synthetic construct; identical to SEQ ID NO: 18
  REGION 1..447: note = BBBB1021 HC2

SEQ ID NO: 78 - protein, 697 aa, synthetic construct
  REGION 1..697: note = BBBB1079 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 79 - protein, 219 aa, synthetic construct; identical to SEQ ID NO: 23
  REGION 1..219: note = BBBB1079 LC

SEQ ID NO: 80 - protein, 445 aa, synthetic construct; identical to SEQ ID NO: 24
  REGION 1..445: note = BBBB1079 HC2

SEQ ID NO: 81 - protein, 697 aa, synthetic construct
  REGION 1..697: note = BBBB1217 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTV

SEQ ID NO: 82 - protein, 218 aa, synthetic construct; identical to SEQ ID NO: 11
  REGION 1..218: note = BBBB1217 LC

SEQ ID NO: 83 - protein, 446 aa, synthetic construct; identical to SEQ ID NO: 27
  REGION 1..446: note = BBBB1217 HC2

SEQ ID NO: 84 - protein, 244 aa, synthetic construct
  REGION 1..244: note = TfR4 (scFv)
EVQLLESGGGLVQPGGSLRLSCAASGFTFSSYAMNWVRQAPGKGLEWVSGISGSGVSTNYADSVKGRFTISRDNSKNTLDLQMNSLRAEDTAVYYCAKEDYDSSGYYPFDYWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 85 - protein, 11 aa, synthetic construct
  REGION 1..11: note = TfR4 HCDR1
GFTFSSYAMNW

SEQ ID NO: 86 - protein, 11 aa, synthetic construct
  REGION 1..11: note = TfR4 HCDR2
SGISGSGVSTN

SEQ ID NO: 87 - protein, 16 aa, synthetic construct
  REGION 1..16: note = TfR4 HCDR3
CAKEDYDSSGYYPFDY

SEQ ID NO: 88 - protein, 7 aa, synthetic construct
  REGION 1..7: note = TfR4 LCDR1
HKLGDKF

SEQ ID NO: 89 - protein, 7 aa, synthetic construct
  REGION 1..7: note = TfR4 LCDR2
QDRKRPS

SEQ ID NO: 90 - protein, 9 aa, synthetic construct
  REGION 1..9: note = TfR4 LCDR3
QTWYSSTVI

SEQ ID NO: 91 - protein, 699 aa, synthetic construct
  REGION 1..699: note = BBBB520 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCAASGFTFSSYAMNWVRQAPGKGLEWVSGISGSGVSTNYADSVKGRFTISRDNSKNTLDLQMNSLRAEDTAVYYCAKEDYDSSGYYPFDYWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 92 - protein, 218 aa, synthetic construct; identical to SEQ ID NO: 11
  REGION 1..218: note = BBBB520 LC

SEQ ID NO: 93 - protein, 449 aa, synthetic construct; identical to SEQ ID NO: 12
  REGION 1..449: note = BBBB520 HC2

SEQ ID NO: 94 - protein, 699 aa, synthetic construct
  REGION 1..699: note = BBBB975 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCAASGFTFSSYAMNWVRQAPGKGLEWVSGISGSGVSTNYADSVKGRFTISRDNSKNTLDLQMNSLRAEDTAVYYCAKEDYDSSGYYPFDYWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 95 - protein, 215 aa, synthetic construct; identical to SEQ ID NO: 14
  REGION 1..215: note = BBBB975 LC

SEQ ID NO: 96 - protein, 449 aa, synthetic construct; identical to SEQ ID NO: 15
  REGION 1..449: note = BBBB975 HC2

SEQ ID NO: 97 - protein, 701 aa, synthetic construct
  REGION 1..701: note = BBBB1026 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCAASGFTFSSYAMNWVRQAPGKGLEWVSGISGSGVSTNYADSVKGRFTISRDNSKNTLDLQMNSLRAEDTAVYYCAKEDYDSSGYYPFDYWGQGTLVTVSSGGSEGKSSGSGSESKSTGGSSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 98 - protein, 214 aa, synthetic construct; identical to SEQ ID NO: 17
  REGION 1..214: note = BBBB1026 LC

SEQ ID NO: 99 - protein, 447 aa, synthetic construct; identical to SEQ ID NO: 46
  REGION 1..447: note = BBBB1026 HC2

SEQ ID NO: 100 - protein, 701 aa, synthetic construct
  REGION 1..701: note = BBBB1024 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCAASGFTFSSYAMNWVRQAPGKGLEWVSGISGSGVSTNYADSVKGRFTISRDNSKNTLDLQMNSLRAEDTAVYYCAKEDYDSSGYYPFDYWGQGTLVTVSSGGSEGKSSGSGSESKSTGGSSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 101 - protein, 214 aa, synthetic construct; identical to SEQ ID NO: 17
  REGION 1..214: note = BBBB1024 LC

SEQ ID NO: 102 - protein, 447 aa, synthetic construct; identical to SEQ ID NO: 18
  REGION 1..447: note = BBBB1024 HC2

SEQ ID NO: 103 - protein, 699 aa, synthetic construct
  REGION 1..699: note = BBBB1082 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCAASGFTFSSYAMNWVRQAPGKGLEWVSGISGSGVSTNYADSVKGRFTISRDNSKNTLDLQMNSLRAEDTAVYYCAKEDYDSSGYYPFDYWGQGTLVTVSSGGSEGKSSGSGSESKSTGGSSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 104 - protein, 219 aa, synthetic construct; identical to SEQ ID NO: 23
  REGION 1..219: note = BBBB1082 LC

SEQ ID NO: 105 - protein, 445 aa, synthetic construct; identical to SEQ ID NO: 24
  REGION 1..445: note = BBBB1082 HC2

SEQ ID NO: 106 - protein, 696 aa, synthetic construct
  REGION 1..696: note = BBBB1218 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSEVQLLESGGGLVQPGGSLRLSCAASGFTFSSYAMNWVRQAPGKGLEWVSGISGSGVSTNYADSVKGRFTISRDNSKNTLDLQMNSLRAEDTAVYYCAKEDYDSSGYYPFDYWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 107 - protein, 218 aa, synthetic construct; identical to SEQ ID NO: 11
  REGION 1..218: note = BBBB1218 LC

SEQ ID NO: 108 - protein, 446 aa, synthetic construct; identical to SEQ ID NO: 27
  REGION 1..446: note = BBBB1218 HC2

SEQ ID NO: 109 - protein, 246 aa, synthetic construct
  REGION 1..246: note = TfR5 (scFv)
QVQLVESGGGVVQPGRSLRLSCAASGFTFSSDAMHWVRQAPGKGLEWVAVIWYDGSNKYSADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYRDSNNFDVLFGGGTKLTVL

SEQ ID NO: 110 - protein, 11 aa, synthetic construct
  REGION 1..11: note = TfR5 HCDR1
GFTFSSDAMHW

SEQ ID NO: 111 - protein, 11 aa, synthetic construct
  REGION 1..11: note = TfR5 HCDR2
AVIWYDGSNKY

SEQ ID NO: 112 - protein, 12 aa, synthetic construct
  REGION 1..12: note = TfR5 HCDR3
CARDRQWLAFDY

SEQ ID NO: 113 - protein, 9 aa, synthetic construct
  REGION 1..9: note = TfR5 LCDR1
SSDVGGYNF

SEQ ID NO: 114 - protein, 7 aa, synthetic construct
  REGION 1..7: note = TfR5 LCDR2
EVSKRPS

SEQ ID NO: 115 - protein, 11 aa, synthetic construct
  REGION 1..11: note = TfR5 LCDR3
SYRDSNNFDVL

SEQ ID NO: 116 - protein, 701 aa, synthetic construct
  REGION 1..701: note = BBBB534 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGVVQPGRSLRLSCAASGFTFSSDAMHWVRQAPGKGLEWVAVIWYDGSNKYSADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYRDSNNFDVLFGGGTKLTVL

SEQ ID NO: 117 - protein, 218 aa, synthetic construct; identical to SEQ ID NO: 11
  REGION 1..218: note = BBBB534 LC

SEQ ID NO: 118 - protein, 449 aa, synthetic construct; identical to SEQ ID NO: 12
  REGION 1..449: note = BBBB534 HC2

SEQ ID NO: 119 - protein, 701 aa, synthetic construct
  REGION 1..701: note = BBBB945 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSQVQLVESGGGVVQPGRSLRLSCAASGFTFSSDAMHWVRQAPGKGLEWVAVIWYDGSNKYSADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYRDSNNFDVLFGGGTKLTVL

SEQ ID NO: 120 - protein, 218 aa, synthetic construct; identical to SEQ ID NO: 11
  REGION 1..218: note = BBBB945 LC

SEQ ID NO: 121 - protein, 449 aa, synthetic construct; identical to SEQ ID NO: 68
  REGION 1..449: note = BBBB945 HC2

SEQ ID NO: 122 - protein, 701 aa, synthetic construct
  REGION 1..701: note = BBBB973 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGVVQPGRSLRLSCAASGFTFSSDAMHWVRQAPGKGLEWVAVIWYDGSNKYSADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYRDSNNFDVLFGGGTKLTVL

SEQ ID NO: 123 - protein, 215 aa, synthetic construct; identical to SEQ ID NO: 14
  REGION 1..215: note = BBBB973 LC

SEQ ID NO: 124 - protein, 449 aa, synthetic construct; identical to SEQ ID NO: 15
  REGION 1..449: note = BBBB973 HC2

SEQ ID NO: 125 - protein, 699 aa, synthetic construct
  REGION 1..699: note = BBBB1035 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGVVQPGRSLRLSCAASGFTFSSDAMHWVRQAPGKGLEWVAVIWYDGSNKYSADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYRDSNNFDVLFGGGTKLTVL

SEQ ID NO: 126 - protein, 214 aa, synthetic construct; identical to SEQ ID NO: 17
  REGION 1..214: note = BBBB1035 LC

SEQ ID NO: 127 - protein, 447 aa, synthetic construct; identical to SEQ ID NO: 46
  REGION 1..447: note = BBBB1035 HC2

SEQ ID NO: 128 - protein, 699 aa, synthetic construct
  REGION 1..699: note = BBBB1033 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLVESGGGVVQPGRSLRLSCAASGFTFSSDAMHWVRQAPGKGLEWVAVIWYDGSNKYSADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYRDSNNFDVLFGGGTKLTVL

SEQ ID NO: 129 - protein, 214 aa, synthetic construct; identical to SEQ ID NO: 17
  REGION 1..214: note = BBBB1033 LC

SEQ ID NO: 130 - protein, 447 aa, synthetic construct; identical to SEQ ID NO: 18
  REGION 1..447: note = BBBB1033 HC2

SEQ ID NO: 131 - protein, 698 aa, synthetic construct
  REGION 1..698: note = BBBB1219 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLVESGGGVVQPGRSLRLSCAASGFTFSSDAMHWVRQAPGKGLEWVAVIWYDGSNKYSADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYRDSNNFDVLFGGGTKLTVL

SEQ ID NO: 132 - protein, 218 aa, synthetic construct; identical to SEQ ID NO: 11
  REGION 1..218: note = BBBB1219 LC

SEQ ID NO: 133 - protein, 446 aa, synthetic construct; identical to SEQ ID NO: 27
  REGION 1..446: note = BBBB1219 HC2

SEQ ID NO: 134 - protein, 244 aa, synthetic construct
  REGION 1..244: note = TfR6 (scFv)
EVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 135 - protein, 11 aa, synthetic construct
  REGION 1..11: note = TfR6 HCDR1
GFTFNNYVMNW

SEQ ID NO: 136 - protein, 8 aa, synthetic construct
  REGION 1..8: note = TfR6 HCDR2
ISGSGGTT

SEQ ID NO: 137 - protein, 16 aa, synthetic construct
  REGION 1..16: note = TfR6 HCDR3
CAKEDDDSTGYYPFDY

SEQ ID NO: 138 - protein, 7 aa, synthetic construct
  REGION 1..7: note = TfR6 LCDR1
KLGDKFV

SEQ ID NO: 139 - protein, 7 aa, synthetic construct
  REGION 1..7: note = TfR6 LCDR2
QDSKRPS

SEQ ID NO: 140 - protein, 9 aa, synthetic construct
  REGION 1..9: note = TfR6 LCDR3
QTWDRSTVV

SEQ ID NO: 141 - protein, 699 aa, synthetic construct
  REGION 1..699: note = BBBB537 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 142 - protein, 218 aa, synthetic construct; identical to SEQ ID NO: 11
  REGION 1..218: note = BBBB537 LC

SEQ ID NO: 143 - protein, 449 aa, synthetic construct; identical to SEQ ID NO: 12
  REGION 1..449: note = BBBB537 HC2

SEQ ID NO: 144 - protein, 699 aa, synthetic construct
  REGION 1..699: note = BBBB989 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSEVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 145 - protein, 218 aa, synthetic construct; identical to SEQ ID NO: 11
  REGION 1..218: note = BBBB989 LC

SEQ ID NO: 146 - protein, 449 aa, synthetic construct; identical to SEQ ID NO: 68
  REGION 1..449: note = BBBB989 HC2

SEQ ID NO: 147 - protein, 699 aa, synthetic construct
  REGION 1..699: note = BBBB977 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 148 - protein, 215 aa, synthetic construct; identical to SEQ ID NO: 14
  REGION 1..215: note = BBBB977 LC

SEQ ID NO: 149 - protein, 449 aa, synthetic construct; identical to SEQ ID NO: 15
  REGION 1..449: note = BBBB977 HC2

SEQ ID NO: 150 - protein, 697 aa, synthetic construct
  REGION 1..697: note = BBBB1038 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 151 - protein, 214 aa, synthetic construct; identical to SEQ ID NO: 17
  REGION 1..214: note = BBBB1038 LC

SEQ ID NO: 152 - protein, 446 aa, synthetic construct
  REGION 1..446: note = BBBB1038 HC2
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSP

SEQ ID NO: 153 - protein, 697 aa, synthetic construct
  REGION 1..697: note = BBBB1036 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 154 - protein, 214 aa, synthetic construct; identical to SEQ ID NO: 17
  REGION 1..214: note = BBBB1036 LC

SEQ ID NO: 155 - protein, 447 aa, synthetic construct; identical to SEQ ID NO: 18
  REGION 1..447: note = BBBB1036 HC2

SEQ ID NO: 156 - protein, 695 aa, synthetic construct
  REGION 1..695: note = BBBB1085 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 157 - protein, 219 aa, synthetic construct; identical to SEQ ID NO: 23
  REGION 1..219: note = BBBB1085 LC

SEQ ID NO: 158 - protein, 445 aa, synthetic construct; identical to SEQ ID NO: 24
  REGION 1..445: note = BBBB1085 HC2

SEQ ID NO: 159 - protein, 696 aa, synthetic construct
  REGION 1..696: note = BBBB1220 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSEVQLLESGGGLVQPGGSLRLSCAASGFTFNNYVMNWVRQAPGKGLEWVSGISGSGGTTYYTDSVKGRFTISRDNSKNTLYLQMHSLRAEDTAIYYCAKEDDDSTGYYPFDYWGQGTLVTVSSGTEGKSSGSGSESKSTSYELTQPPSASVSPGQTASITCSGDKLGDKFVCWYQQKPGQSPVLAIYQDSKRPSGIPERFSGSNSGNTATLTISGTQAMDEADYYCQTWDRSTVVFGGGTKLTVL

SEQ ID NO: 160 - protein, 218 aa, synthetic construct; identical to SEQ ID NO: 11
  REGION 1..218: note = BBBB1220 LC

SEQ ID NO: 161 - protein, 446 aa, synthetic construct; identical to SEQ ID NO: 27
  REGION 1..446: note = BBBB1220 HC2

SEQ ID NO: 162 - protein, 244 aa, synthetic construct
  REGION 1..244: note = TfR7
EVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 163 - protein, 11 aa, synthetic construct
  REGION 1..11: note = TfR7 HCDR1
GFTFSSYAMNW

SEQ ID NO: 164 - protein, 8 aa, synthetic construct
  REGION 1..8: note = TfR7 HCDR2
ISGSGGHT

SEQ ID NO: 165 - protein, 16 aa, synthetic construct
  REGION 1..16: note = TfR7 HCDR3
CAREGYDSSGYNPFDY

SEQ ID NO: 166 - protein, 7 aa, synthetic construct
  REGION 1..7: note = TfR7 LCDR1
KLGDKYA

SEQ ID NO: 167 - protein, 7 aa, synthetic construct
  REGION 1..7: note = TfR7 LCDR2
QDSKRPS

SEQ ID NO: 168 - protein, 9 aa, synthetic construct
  REGION 1..9: note = TfR7 LCDR3
QAWDSSTVV

SEQ ID NO: 169 - protein, 699 aa, synthetic construct
  REGION 1..699: note = BBBB543 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 170 - protein, 218 aa, synthetic construct; identical to SEQ ID NO: 11
  REGION 1..218: note = BBBB543 LC

SEQ ID NO: 171 - protein, 449 aa, synthetic construct; identical to SEQ ID NO: 12
  REGION 1..449: note = BBBB543 HC2

SEQ ID NO: 172 - protein, 699 aa, synthetic construct
  REGION 1..699: note = BBBB1112 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 173 - protein, 218 aa, synthetic construct; identical to SEQ ID NO: 11
  REGION 1..218: note = BBBB1112 LC

SEQ ID NO: 174 - protein, 449 aa, synthetic construct; identical to SEQ ID NO: 12
  REGION 1..449: note = BBBB1112 HC2

SEQ ID NO: 175 - protein, 699 aa, synthetic construct
  REGION 1..699: note = BBBB969 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 176 - protein, 215 aa, synthetic construct; identical to SEQ ID NO: 14
  REGION 1..215: note = BBBB969 LC

SEQ ID NO: 177 - protein, 449 aa, synthetic construct; identical to SEQ ID NO: 15
  REGION 1..449: note = BBBB969 HC2

SEQ ID NO: 178 - protein, 697 aa, synthetic construct
  REGION 1..697: note = BBBB1048 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 179 - protein, 214 aa, synthetic construct; identical to SEQ ID NO: 17
  REGION 1..214: note = BBBB1048 LC

SEQ ID NO: 180 - protein, 447 aa, synthetic construct; identical to SEQ ID NO: 46
  REGION 1..447: note = BBBB1048 HC2

SEQ ID NO: 181 - protein, 697 aa, synthetic construct
  REGION 1..697: note = BBBB1046 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 182 - protein, 214 aa, synthetic construct; identical to SEQ ID NO: 17
  REGION 1..214: note = BBBB1046 LC

SEQ ID NO: 183 - protein, 447 aa, synthetic construct; identical to SEQ ID NO: 18
  REGION 1..447: note = BBBB1046 HC2

SEQ ID NO: 184 - protein, 695 aa, synthetic construct
  REGION 1..695: note = BBBB1088 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 185 - protein, 219 aa, synthetic construct; identical to SEQ ID NO: 23
  REGION 1..219: note = BBBB1088 LC

SEQ ID NO: 186 - protein, 445 aa, synthetic construct; identical to SEQ ID NO: 24
  REGION 1..445: note = BBBB1088 HC2

SEQ ID NO: 187 - protein, 696 aa, synthetic construct
  REGION 1..696: note = BBBB1221 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 188 - protein, 218 aa, synthetic construct; identical to SEQ ID NO: 11
  REGION 1..218: note = BBBB1221 LC

SEQ ID NO: 189 - protein, 446 aa, synthetic construct; identical to SEQ ID NO: 27
  REGION 1..446: note = BBBB1221 HC2

SEQ ID NO: 190 - protein, 247 aa, synthetic construct
  REGION 1..247: note = TfR8
QLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTVL

SEQ ID NO: 191 - protein, 10 aa, synthetic construct
  REGION 1..10: note = TfR8 HCDR1
GGSISSTSYY

SEQ ID NO: 192 - protein, 7 aa, synthetic construct
  REGION 1..7: note = TfR8 HCDR2
IYYSGNT

SEQ ID NO: 193 - protein, 15 aa, synthetic construct
  REGION 1..15: note = TfR8 HCDR3
ARHDWYGGSYGWFDP

SEQ ID NO: 194 - protein, 6 aa, synthetic construct
  REGION 1..6: note = TfR8 LCDR1
SLRSYY

SEQ ID NO: 195 - protein, 7 aa, synthetic construct
  REGION 1..7: note = TfR8 LCDR2
AKNNRPS

SEQ ID NO: 196 - protein, 11 aa, synthetic construct
  REGION 1..11: note = TfR8 LCDR3
NSRDSSGNHMV

SEQ ID NO: 197 - protein, 702 aa, synthetic construct
  REGION 1..702: note = BBBB556 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTVL

SEQ ID NO: 198 - protein, 218 aa, synthetic construct; identical to SEQ ID NO: 11
  REGION 1..218: note = BBBB556 LC

SEQ ID NO: 199 - protein, 449 aa, synthetic construct; identical to SEQ ID NO: 12
  REGION 1..449: note = BBBB556 HC2

SEQ ID NO: 200 - protein, 702 aa, synthetic construct
  REGION 1..702: note = BBBB993 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSQLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTVL

SEQ ID NO: 201 - protein, 218 aa, synthetic construct; identical to SEQ ID NO: 11
  REGION 1..218: note = BBBB993 LC

SEQ ID NO: 202 - protein, 449 aa, synthetic construct; identical to SEQ ID NO: 68
  REGION 1..449: note = BBBB993 HC2

SEQ ID NO: 203 - protein, 702 aa, synthetic construct
  REGION 1..702: note = BBBB983 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTVL

SEQ ID NO: 204 - protein, 215 aa, synthetic construct; identical to SEQ ID NO: 14
  REGION 1..215: note = BBBB983 LC

SEQ ID NO: 205 - protein, 449 aa, synthetic construct; identical to SEQ ID NO: 15
  REGION 1..449: note = BBBB983 HC2

SEQ ID NO: 206 - protein, 700 aa, synthetic construct
  REGION 1..700: note = BBBB1051 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTVL

SEQ ID NO: 207 - protein, 214 aa, synthetic construct; identical to SEQ ID NO: 17
  REGION 1..214: note = BBBB1051 LC

SEQ ID NO: 208 - protein, 447 aa, synthetic construct; identical to SEQ ID NO: 46
  REGION 1..447: note = BBBB1051 HC2

SEQ ID NO: 209 - protein, 700 aa, synthetic construct
  REGION 1..700: note = BBBB1049 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTVL

SEQ ID NO: 210 - protein, 214 aa, synthetic construct; identical to SEQ ID NO: 17
  REGION 1..214: note = BBBB1049 LC

SEQ ID NO: 211 - protein, 447 aa, synthetic construct; identical to SEQ ID NO: 18
  REGION 1..447: note = BBBB1049 HC2

SEQ ID NO: 212 - protein, 698 aa, synthetic construct
  REGION 1..698: note = BBBB1091 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTVL

SEQ ID NO: 213 - protein, 219 aa, synthetic construct; identical to SEQ ID NO: 23
  REGION 1..219: note = BBBB1091 LC

SEQ ID NO: 214 - protein, 445 aa, synthetic construct; identical to SEQ ID NO: 24
  REGION 1..445: note = BBBB1091 HC2

SEQ ID NO: 215 - protein, 698 aa, synthetic construct
  REGION 1..698: note = BBBB1222 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQLRLQESGPGLVKPSETLSLTCTVSGGSISSTSYYWGWIRQPPGKGLGWIGSIYYSGNTYYNPSLKSRVTISVDTSKNQFSLTLSSVTAADTAVYYCARHDWYGGSYGWFDPWGQGTLVAVSSGTEGKSSGSGSESKSTSSELTQDPAVSVALGQTVRITCQGDSLRSYYASWYQQKPGQAPVLVIYAKNNRPSGIPDRFSGSSSGNTASLTITGAQAEDEADYYCNSRDSSGNHMVFGGGTTLTV

SEQ ID NO: 216 - protein, 218 aa, synthetic construct; identical to SEQ ID NO: 11
  REGION 1..218: note = BBBB1222 LC

SEQ ID NO: 217 - protein, 446 aa, synthetic construct; identical to SEQ ID NO: 27
  REGION 1..446: note = BBBB1222 HC2

SEQ ID NO: 218 - protein, 243 aa, synthetic construct
  REGION 1..243: note = TfR9
QVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 219 - protein, 8 aa, synthetic construct
  REGION 1..8: note = TfR9 HCDR1
GGSFSGYY

SEQ ID NO: 220 - protein, 7 aa, synthetic construct
  REGION 1..7: note = TfR9 HCDR2
FIHSGST

SEQ ID NO: 221 - protein, 15 aa, synthetic construct
  REGION 1..15: note = TfR9 HCDR3
ARGSMDSSGFYSFDF

SEQ ID NO: 222 - protein, 6 aa, synthetic construct
  REGION 1..6: note = TfR9 LCDR1
KLGDKF

SEQ ID NO: 223 - protein, 6 aa, synthetic construct
  REGION 1..6: note = TfR9 LCDR2
QDRKRP

SEQ ID NO: 224 - protein, 9 aa, synthetic construct
  REGION 1..9: note = TfR9 LCDR3
QTWYSSTVI

SEQ ID NO: 225 - protein, 698 aa, synthetic construct
  REGION 1..698: note = BBBB557 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 226 - protein, 218 aa, synthetic construct; identical to SEQ ID NO: 11
  REGION 1..218: note = BBBB557 LC

SEQ ID NO: 227 - protein, 449 aa, synthetic construct; identical to SEQ ID NO: 12
  REGION 1..449: note = BBBB557 HC2

SEQ ID NO: 228 - protein, 698 aa, synthetic construct
  REGION 1..698: note = BBBB970 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 229 - protein, 215 aa, synthetic construct; identical to SEQ ID NO: 14
  REGION 1..215: note = BBBB970 LC

SEQ ID NO: 230 - protein, 449 aa, synthetic construct; identical to SEQ ID NO: 15
  REGION 1..449: note = BBBB970 HC2

SEQ ID NO: 231 - protein, 696 aa, synthetic construct
  REGION 1..696: note = BBBB1055 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 232 - protein, 214 aa, synthetic construct; identical to SEQ ID NO: 17
  REGION 1..214: note = BBBB1055 LC

SEQ ID NO: 233 - protein, 447 aa, synthetic construct; identical to SEQ ID NO: 46
  REGION 1..447: note = BBBB1055 HC2

SEQ ID NO: 234 - protein, 696 aa, synthetic construct
  REGION 1..696: note = BBBB1053 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 235 - protein, 214 aa, synthetic construct; identical to SEQ ID NO: 17
  REGION 1..214: note = BBBB1053 LC

SEQ ID NO: 236 - protein, 447 aa, synthetic construct; identical to SEQ ID NO: 18
  REGION 1..447: note = BBBB1053 HC2

SEQ ID NO: 237 - protein, 694 aa, synthetic construct
  REGION 1..694: note = BBBB1094 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 238 - protein, 219 aa, synthetic construct; identical to SEQ ID NO: 23
  REGION 1..219: note = BBBB1094 LC

SEQ ID NO: 239 - protein, 445 aa, synthetic construct; identical to SEQ ID NO: 24
  REGION 1..445: note = BBBB1094 HC2

SEQ ID NO: 240 - protein, 695 aa, synthetic construct
  REGION 1..695: note = BBBB1223 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 241 - protein, 218 aa, synthetic construct; identical to SEQ ID NO: 11
  REGION 1..218: note = BBBB1223 LC

SEQ ID NO: 242 - protein, 446 aa, synthetic construct; identical to SEQ ID NO: 27
  REGION 1..446: note = BBBB1223 HC2

SEQ ID NO: 243 - protein, 243 aa, synthetic construct
  REGION 1..243: note = TfR10
EVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGRGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 244 - protein, 8 aa, synthetic construct
  REGION 1..8: note = TfR10 HCDR1
GYSFTSYW

SEQ ID NO: 245 - protein, 8 aa, synthetic construct
  REGION 1..8: note = TfR10 HCDR2
IDPSDSYT

SEQ ID NO: 246 - protein, 13 aa, synthetic construct
  REGION 1..13: note = TfR10 HCDR3
ARMYKGRGHLFDY

SEQ ID NO: 247 - protein, 6 aa, synthetic construct
  REGION 1..6: note = TfR10 LCDR1
QSVSKA

SEQ ID NO: 248 - protein, 7 aa, synthetic construct
  REGION 1..7: note = TfR10 LCDR2
AASNRAT

SEQ ID NO: 249 - protein, 9 aa, synthetic construct
  REGION 1..9: note = TfR10 LCDR3
QQYYRAPYT

SEQ ID NO: 250 - protein, 698 aa, synthetic construct
  REGION 1..698: note = BBBB354 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGRGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 251 - protein, 218 aa, synthetic construct; identical to SEQ ID NO: 11
  REGION 1..218: note = BBBB354 LC

SEQ ID NO: 252 - protein, 449 aa, synthetic construct; identical to SEQ ID NO: 12
  REGION 1..449: note = BBBB354 HC2

SEQ ID NO: 253 - protein, 698 aa, synthetic construct
  REGION 1..698: note = BBBB932 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSEVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGRGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 254 - protein, 218 aa, synthetic construct; identical to SEQ ID NO: 11
  REGION 1..218: note = BBBB932 LC

SEQ ID NO: 255 - protein, 449 aa, synthetic construct; identical to SEQ ID NO: 68
  REGION 1..449: note = BBBB932 HC2

SEQ ID NO: 256 - protein, 698 aa, synthetic construct
  REGION 1..698: note = BBBB383 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGRGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 257 - protein, 215 aa, synthetic construct; identical to SEQ ID NO: 14
  REGION 1..215: note = BBBB383 LC

SEQ ID NO: 258 - protein, 449 aa, synthetic construct; identical to SEQ ID NO: 15
  REGION 1..449: note = BBBB383 HC2

SEQ ID NO: 259 - protein, 695 aa, synthetic construct
  REGION 1..695: note = BBBB1224 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGGGSGGSEVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGRGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 260 - protein, 218 aa, synthetic construct; identical to SEQ ID NO: 11
  REGION 1..218: note = BBBB1224 LC

SEQ ID NO: 261 - protein, 446 aa, synthetic construct; identical to SEQ ID NO: 27
  REGION 1..446: note = BBBB1224 HC2

SEQ ID NO: 262 - protein, 243 aa, synthetic construct
  REGION 1..243: note = TfR 11 (svFc)
EVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGHGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 263 - protein, 8 aa, synthetic construct
  REGION 1..8: note = TfR 11 HCDR1
GYSFTSYW

SEQ ID NO: 264 - protein, 8 aa, synthetic construct
  REGION 1..8: note = TfR 11 HCDR2
IDPSDSYT

SEQ ID NO: 265 - protein, 13 aa, synthetic construct
  REGION 1..13: note = TfR 11 HCDR3
ARMYKGHGHLFDY

SEQ ID NO: 266 - protein, 6 aa, synthetic construct
  REGION 1..6: note = TfR 11 LCDR1
QSVSKA

SEQ ID NO: 267 - protein, 7 aa, synthetic construct
  REGION 1..7: note = TfR 11 LCDR2
AASNRAT

SEQ ID NO: 268 - protein, 9 aa, synthetic construct
  REGION 1..9: note = TfR 11 LCDR3
QQYYRAPYT

SEQ ID NO: 269 - protein, 698 aa, synthetic construct
  REGION 1..698: note = BBBB368 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGHGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 270 - protein, 218 aa, synthetic construct; identical to SEQ ID NO: 11
  REGION 1..218: note = BBBB368 LC

SEQ ID NO: 271 - protein, 449 aa, synthetic construct; identical to SEQ ID NO: 12
  REGION 1..449: note = BBBB368 HC2

SEQ ID NO: 272 - protein, 698 aa, synthetic construct
  REGION 1..698: note = BBBB934 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGSGGSEVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGHGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 273 - protein, 218 aa, synthetic construct; identical to SEQ ID NO: 11
  REGION 1..218: note = BBBB934 LC

SEQ ID NO: 274 - protein, 449 aa, synthetic construct; identical to SEQ ID NO: 68
  REGION 1..449: note = BBBB934 HC2

SEQ ID NO: 275 - protein, 698 aa, synthetic construct
  REGION 1..698: note = BBBB426 HC1
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVQSGAEVKKPGESLKISCKGSGYSFTSYWIGWVRQMPGKGLEWMGIIDPSDSYTRYSPSFQGQVTISADKSISTAYLQWSSLKASDTAMYYCARMYKGHGHLFDYWGQGTLVTVSSGTEGKSSGSGSESKSTEIVLTQSPATLSLSPGERATLSCRASQSVSKALAWYQQKPGQAPRLLIYAASNRATGIPARFSGSGSGTDFTLTISSLEPEDFAVYYCQQYYRAPYTFGQGTKVEIK

SEQ ID NO: 276 - protein, 215 aa, synthetic construct; identical to SEQ ID NO: 14
  REGION 1..215: note = BBBB426 LC

SEQ ID NO: 277 - protein, 449 aa, synthetic construct; identical to SEQ ID NO: 15
  REGION 1..449: note = BBBB426 HC2

SEQ ID NO: 278 - protein, 243 aa, synthetic construct
  REGION 1..243: note = TfR 12
QVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFASWYQQKPGQSPVLVIYQDRKRPSGIPERFSGSNSGNTATLTISGTQAMDEADYYCQTWYSSTVIFGGGTKLTVL

SEQ ID NO: 279 - protein, 8 aa, synthetic construct
  REGION 1..8: note = TfR 12 HCDR1
GGSFSGYY

SEQ ID NO: 280 - protein, 7 aa, synthetic construct
  REGION 1..7: note = TfR 12 HCDR2
FIHSGST

SEQ ID NO: 281 - protein, 15 aa, synthetic construct
  REGION 1..15: note = TfR 12 HCDR3
ARGSMDSSGFYSFDF

SEQ ID NO: 282 - protein, 6 aa, synthetic construct
  REGION 1..6: note = TfR 12 LCDR1
KLGDKF

SEQ ID NO: 283 - protein, 6 aa, synthetic construct
  REGION 1..6: note = TfR 12 LCDR2
QDRKRP

SEQ ID NO: 284 - protein, 9 aa, synthetic construct
  REGION 1..9: note = TfR 12 LCDR3
QTWYSSTVI

SEQ ID NO: 285 - protein, 696 aa, synthetic construct
  REGION 1..696: note = BBBB1136 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFASWYQQKPGQSPVLVIYQDRKRPSGIPERFSGSNSGNTATLTISGTQAMDEADYYCQTWYSSTVIFGGGTKLTVL

SEQ ID NO: 286 - protein, 214 aa, synthetic construct; identical to SEQ ID NO: 17
  REGION 1..214: note = BBBB1136 LC

SEQ ID NO: 287 - protein, 447 aa, synthetic construct; identical to SEQ ID NO: 46
  REGION 1..447: note = BBBB1136 HC2

SEQ ID NO: 288 - protein, 696 aa, synthetic construct
  REGION 1..696: note = BBBB1134 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAQVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFASWYQQKPGQSPVLVIYQDRKRPSGIPERFSGSNSGNTATLTISGTQAMDEADYYCQTWYSSTVIFGGGTKLTVL

SEQ ID NO: 289 - protein, 214 aa, synthetic construct; identical to SEQ ID NO: 17
  REGION 1..214: note = BBBB1134 LC

SEQ ID NO: 290 - protein, 447 aa, synthetic construct; identical to SEQ ID NO: 18
  REGION 1..447: note = BBBB1134 HC2

SEQ ID NO: 291 - protein, 244 aa, synthetic construct
  REGION 1..244: note = TfR 13
EVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 292 - protein, 11 aa, synthetic construct
  REGION 1..11: note = TfR 13 HCDR1
GFTFSSYAMNW

SEQ ID NO: 293 - protein, 8 aa, synthetic construct
  REGION 1..8: note = TfR 13 HCDR2
ISGSGGHT

SEQ ID NO: 294 - protein, 15 aa, synthetic construct
  REGION 1..15: note = TfR 13 HCDR3
AREGYDSSGYNPFDY

SEQ ID NO: 295 - protein, 7 aa, synthetic construct
  REGION 1..7: note = TfR 13 LCDR1
KLGDKYA

SEQ ID NO: 296 - protein, 7 aa, synthetic construct
  REGION 1..7: note = TfR 13 LCDR2
QDSKRPS

SEQ ID NO: 297 - protein, 9 aa, synthetic construct
  REGION 1..9: note = TfR 13 LCDR3
QAWDSSTVV

SEQ ID NO: 298 - protein, 697 aa, synthetic construct
  REGION 1..697: note = BBBB1133 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 299 - protein, 214 aa, synthetic construct; identical to SEQ ID NO: 17
  REGION 1..214: note = BBBB1133 LC

SEQ ID NO: 300 - protein, 447 aa, synthetic construct; identical to SEQ ID NO: 46
  REGION 1..447: note = BBBB1133 HC2

SEQ ID NO: 301 - protein, 697 aa, synthetic construct
  REGION 1..697: note = BBBB1131 HC1
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 302 - protein, 214 aa, synthetic construct; identical to SEQ ID NO: 17
  REGION 1..214: note = BBBB1131 LC

SEQ ID NO: 303 - protein, 447 aa, synthetic construct; identical to SEQ ID NO: 18
  REGION 1..447: note = BBBB1131 HC2

SEQ ID NO: 304 - protein, 695 aa, synthetic construct
  REGION 1..695: note = BBBB1166 HC1
QVQLVQSGAEVKKPGASVKVSCKASGHVFTSYDMYWVRQAPGQGLEWIGYIDSDSGDTSYNQKFKGRVTLTVDTSTSTVYMELSSLRSEDTAVYYCAYYRYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGGGAGGAEVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 305 - protein, 219 aa, synthetic construct
  REGION 1..219: note = BBBB1166 LC
DVVMTQSPLSLPVTLGQPASISCKSSQSLLDSRAKTYLTWLQQRPGQSPRRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 306 - protein, 445 aa, synthetic construct; identical to SEQ ID NO: 24
  REGION 1..445: note = BBBB1166 HC2

SEQ ID NO: 307 - protein, 449 aa, synthetic construct; identical to SEQ ID NO: 15
  REGION 1..449: note = BBBB456 HC1

SEQ ID NO: 308 - protein, 215 aa, synthetic construct; identical to SEQ ID NO: 14
  REGION 1..215: note = BBBB456 LC

SEQ ID NO: 309 - protein, 449 aa, synthetic construct
  REGION 1..449: note = BBBB456 HC2
QVQLVQSGAEVKKPGSSVKVSCKASGGTLSHYGVSWVRQAPGQGLEWMGNIIPGIGTANYAQKFQGRVTITADESTSTAYMELSSLRSEDTAVYYCARSGGTKYGMLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 310 - protein, 448 aa, synthetic construct
  REGION 1..448: note = PT1B844 HC
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 214 aa, synthetic construct; identical to SEQ ID NO: 17
  REGION 1..214: note = PT1B844 & PT1B916 LC

SEQ ID NO: 312 - protein, 6 aa, synthetic construct
  REGION 1..6: note = Linker 1
GGSGGS

SEQ ID NO: 313 - protein, 6 aa, synthetic construct
  REGION 1..6: note = Linker 2
GGAGGA

SEQ ID NO: 314 - protein, 16 aa, synthetic construct
  REGION 1..16: note = Linker 3
GTEGKSSGSGSESKST

SEQ ID NO: 315 - protein, 448 aa, synthetic construct
  REGION 1..448: note = PT1B916 HC
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 129 aa, synthetic construct
  REGION 1..129: note = TfR 14
QVQLVESGGGLVQTGGSLRLSCAASGLTFSNYAMGWFRQAPGKKREFVAHIGGSGGTWRCADSVKGRFTISRDNAKNMVYLQMNSLNTEDTAIYYCAADQRAGSYSSGWYTRSSDSLYWGQGTQVTVSS

SEQ ID NO: 317 - protein, 8 aa, synthetic construct
  REGION 1..8: note = TfR 14 HCDR1
GLTFSNYA

SEQ ID NO: 318 - protein, 8 aa, synthetic construct
  REGION 1..8: note = TfR 14 HCDR2
IGGSGGTW

SEQ ID NO: 319 - protein, 22 aa, synthetic construct
  REGION 1..22: note = TfR 14 HCDR3
AADQRAGSYSSGWYTRSSDSLY

SEQ ID NO: 320 - protein, 584 aa, synthetic construct
  REGION 1..584: note = BBBB432 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVQTGGSLRLSCAASGLTFSNYAMGWFRQAPGKKREFVAHIGGSGGTWRCADSVKGRFTISRDNAKNMVYLQMNSLNTEDTAIYYCAADQRAGSYSSGWYTRSSDSLYWGQGTQVTVSS

SEQ ID NO: 321 - protein, 218 aa, synthetic construct; identical to SEQ ID NO: 11
  REGION 1..218: note = BBBB432 LC

SEQ ID NO: 322 - protein, 449 aa, synthetic construct; identical to SEQ ID NO: 12
  REGION 1..449: note = BBBB432 HC2

SEQ ID NO: 323 - protein, 129 aa, synthetic construct
  REGION 1..129: note = TfR 15
QVQLVESGGGLVQAGGSLRLSCAASGLTFSSYVMGWFRQAPGKEREFVAHINGDGKFRYADSVKGWFTISRDNANNMVYLQMNSLNTEDTAIYYCASDQRAGSLSSGWYSRRSYDTLYWGQGTQVTVSS

SEQ ID NO: 324 - protein, 8 aa, synthetic construct
  REGION 1..8: note = TfR 15 HCDR1
GLTFSSYV

SEQ ID NO: 325 - protein, 7 aa, synthetic construct
  REGION 1..7: note = TfR 15 HCDR2
INGDGKF

SEQ ID NO: 326 - protein, 23 aa, synthetic construct
  REGION 1..23: note = TfR 15 HCDR3
ASDQRAGSLSSGWYSRRSYDTLY

SEQ ID NO: 327 - protein, 584 aa, synthetic construct
  REGION 1..584: note = BBBB435 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVQAGGSLRLSCAASGLTFSSYVMGWFRQAPGKEREFVAHINGDGKFRYADSVKGWFTISRDNANNMVYLQMNSLNTEDTAIYYCASDQRAGSLSSGWYSRRSYDTLYWGQGTQVTVSS

SEQ ID NO: 328 - protein, 218 aa, synthetic construct; identical to SEQ ID NO: 11
  REGION 1..218: note = BBBB435 LC

SEQ ID NO: 329 - protein, 449 aa, synthetic construct; identical to SEQ ID NO: 12
  REGION 1..449: note = BBBB435 HC2

SEQ ID NO: 330 - protein, 128 aa, synthetic construct
  REGION 1..128: note = TFR 16
QVQLVESGGGLVQPGGSLRLSCAASGFTFSSYAMGWFRQAPGKEREFVAQISWSGRWRYADSVKGRFTISRDNANNMVYLQMNSLNTEDTAIYYCVSDQRPGTLSSGWYSRSSDTLYWGQGTKVTVSS

SEQ ID NO: 331 - protein, 8 aa, synthetic construct
  REGION 1..8: note = TfR 16 HCDR1
GFTFSSYA

SEQ ID NO: 332 - protein, 7 aa, synthetic construct
  REGION 1..7: note = TfR 16 HCDR2
ISWSGRW

SEQ ID NO: 333 - protein, 22 aa, synthetic construct
  REGION 1..22: note = TfR 16 HCDR3
VSDQRPGTLSSGWYSRSSDTLY

SEQ ID NO: 334 - protein, 583 aa, synthetic construct
  REGION 1..583: note = BBBB436 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVQPGGSLRLSCAASGFTFSSYAMGWFRQAPGKEREFVAQISWSGRWRYADSVKGRFTISRDNANNMVYLQMNSLNTEDTAIYYCVSDQRPGTLSSGWYSRSSDTLYWGQGTKVTVSS

SEQ ID NO: 335 - protein, 218 aa, synthetic construct; identical to SEQ ID NO: 11
  REGION 1..218: note = BBBB436 LC

SEQ ID NO: 336 - protein, 449 aa, synthetic construct; identical to SEQ ID NO: 12
  REGION 1..449: note = BBBB436 HC2

SEQ ID NO: 337 - protein, 115 aa, synthetic construct
  REGION 1..115: note = TfR 17
QVHLVESGGGLVQAGGSLTLSCAASVRISSANVVGWYREAPGKQRELVAQSISGGDPYYLNSVKGRFTISRDNAKNTVYLQMNSLKPEDTAVYYCNYWNEGIRYGGQGTQVTVSS

SEQ ID NO: 338 - protein, 9 aa, synthetic construct
  REGION 1..9: note = TfR 17 HCDR1
VRISSANVV

SEQ ID NO: 339 - protein, 7 aa, synthetic construct
  REGION 1..7: note = TfR 17 HCDR2
SISGGDP

SEQ ID NO: 340 - protein, 9 aa, synthetic construct
  REGION 1..9: note = TfR 17 HCDR3
NYWNEGIRY

SEQ ID NO: 341 - protein, 570 aa, synthetic construct
  REGION 1..570: note = BBBB439 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVHLVESGGGLVQAGGSLTLSCAASVRISSANVVGWYREAPGKQRELVAQSISGGDPYYLNSVKGRFTISRDNAKNTVYLQMNSLKPEDTAVYYCNYWNEGIRYGGQGTQVTVSS

SEQ ID NO: 342 - protein, 218 aa, synthetic construct; identical to SEQ ID NO: 11
  REGION 1..218: note = BBBB439 LC

SEQ ID NO: 343 - protein, 449 aa, synthetic construct; identical to SEQ ID NO: 12
  REGION 1..449: note = BBBB439 HC2

SEQ ID NO: 344 - protein, 249 aa, synthetic construct
  REGION 1..249: note = TfR 18
EVQLVESGGGLVQPGGSLRLSCAASGFTFSSYWMHWVRQAPGKGLVWVSRINNIGNSRSYADSVKGRFTISRDNAKNTLYLQMNSLSAEDTAVYYCARAGNWDRDTFDIWGQGTMVTVSSGTEGKSSGSGSESKSTDIVMTQSPDSLAVSLGERATINCKSSQSVLYSSNNKIYLAWYQQKPGQPPKLLIYWASTRESGVPDRFSGSGSGTDFTLTISSLQAEDVAVYYCQQYYSTPYTFGQGTKLEIK

SEQ ID NO: 345 - protein, 8 aa, synthetic construct
  REGION 1..8: note = TfR 18 HCDR1
GFTFSSYW

SEQ ID NO: 346 - protein, 8 aa, synthetic construct
  REGION 1..8: note = TfR 18 HCDR2
INNIGNSR

SEQ ID NO: 347 - protein, 13 aa, synthetic construct
  REGION 1..13: note = TfR 18 HCDR3
ARAGNWDRDTFDI

SEQ ID NO: 348 - protein, 12 aa, synthetic construct
  REGION 1..12: note = TfR 18 LCDR1
QSVLYSSNNKIY

SEQ ID NO: 349 - skipped

SEQ ID NO: 350 - protein, 9 aa, synthetic construct
  REGION 1..9: note = TfR 18 LCDR3
QQYYSTPYT

SEQ ID NO: 351 - protein, 704 aa, synthetic construct
  REGION 1..704: note = BBBB459 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVQPGGSLRLSCAASGFTFSSYWMHWVRQAPGKGLVWVSRINNIGNSRSYADSVKGRFTISRDNAKNTLYLQMNSLSAEDTAVYYCARAGNWDRDTFDIWGQGTMVTVSSGTEGKSSGSGSESKSTDIVMTQSPDSLAVSLGERATINCKSSQSVLYSSNNKIYLAWYQQKPGQPPKLLIYWASTRESGVPDRFSGSGSGTDFTLTISSLQAEDVAVYYCQQYYSTPYTFGQGTKLEIK

SEQ ID NO: 352 - protein, 218 aa, synthetic construct; identical to SEQ ID NO: 11
  REGION 1..218: note = BBBB459 LC

SEQ ID NO: 353 - protein, 449 aa, synthetic construct; identical to SEQ ID NO: 12
  REGION 1..449: note = BBBB459 HC2

SEQ ID NO: 354 - protein, 247 aa, synthetic construct
  REGION 1..247: note = TfR 19
EVQLVESGGGLVKPGGSLRLSCAASGFTFSRYSMNWVRQAPGKGLEWVSSISSSSTNIYYADSVKGRFTLSRDNAKNSLYLQMNSLRAEDTAVYYCARDYMWKVFDYWGQGTLVTVSSGTEGKSSGSGSESKSTDIVMTQTPLSLPVTPGEPASISCRSSQSLLDSDDGNIFLDWYLQKPGQSPQLLIYTLSYRASGVPDRFSGSGSGTDFTLKISRVEAEDVGVYYCMQRIEFPITFGQGTRLEIK

SEQ ID NO: 355 - protein, 8 aa, synthetic construct
  REGION 1..8: note = TfR 19 HCDR1
GFTFSRYS

SEQ ID NO: 356 - protein, 8 aa, synthetic construct
  REGION 1..8: note = TfR 19 HCDR2
ISSSSTNI

SEQ ID NO: 357 - protein, 11 aa, synthetic construct
  REGION 1..11: note = TfR 19 HCDR3
ARDYMWKVFDY

SEQ ID NO: 358 - protein, 12 aa, synthetic construct
  REGION 1..12: note = TfR 19 LCDR1
QSLLDSDDGNIF

SEQ ID NO: 359 - skipped

SEQ ID NO: 360 - protein, 9 aa, synthetic construct
  REGION 1..9: note = TfR 19 LCDR3
MQRIEFPIT

SEQ ID NO: 361 - protein, 702 aa, synthetic construct
  REGION 1..702: note = BBBB464 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVKPGGSLRLSCAASGFTFSRYSMNWVRQAPGKGLEWVSSISSSSTNIYYADSVKGRFTLSRDNAKNSLYLQMNSLRAEDTAVYYCARDYMWKVFDYWGQGTLVTVSSGTEGKSSGSGSESKSTDIVMTQTPLSLPVTPGEPASISCRSSQSLLDSDDGNIFLDWYLQKPGQSPQLLIYTLSYRASGVPDRFSGSGSGTDFTLKISRVEAEDVGVYYCMQRIEFPITFGQGTRLEIK

SEQ ID NO: 362 - protein, 218 aa, synthetic construct; identical to SEQ ID NO: 11
  REGION 1..218: note = BBBB464 LC

SEQ ID NO: 363 - protein, 449 aa, synthetic construct; identical to SEQ ID NO: 12
  REGION 1..449: note = BBBB464 HC2

SEQ ID NO: 364 - protein, 247 aa, synthetic construct
  REGION 1..247: note = TfR 20
EVQLVESGGGLVKPGGSLRLSCAASGFTFSRYSMNWVRQAPGKGLEWVSSISSSSTNINYADSVRGRFTISRDNAKNSLYLQMNSLRAEDTAVYYCAREYMWKVFDYWGQGTLVTVSSGTEGKSSGSGSESKSTDIVMTQTPLSLPVTPGEPASISCRSSQSLLDSDDGNIFLDWFLQKPGQSPQLLIYTVSYRASGVPDRFSGSGSGTDFTLKISRVEAEDVGVYYCMQRIEFPITFGQGTRLEIK

SEQ ID NO: 365 - protein, 8 aa, synthetic construct
  REGION 1..8: note = TfR 20 HCDR1
GFTFSRYS

SEQ ID NO: 366 - protein, 8 aa, synthetic construct
  REGION 1..8: note = TfR 20 HCDR2
ISSSSTNI

SEQ ID NO: 367 - protein, 11 aa, synthetic construct
  REGION 1..11: note = TfR 20 HCDR3
AREYMWKVFDY

SEQ ID NO: 368 - protein, 12 aa, synthetic construct
  REGION 1..12: note = TfR 20 LCDR1
QSLLDSDDGNIF

SEQ ID NO: 369 - skipped

SEQ ID NO: 370 - protein, 9 aa, synthetic construct
  REGION 1..9: note = TfR 20 LCDR3
MQRIEFPIT

SEQ ID NO: 371 - protein, 702 aa, synthetic construct
  REGION 1..702: note = BBBB467 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVKPGGSLRLSCAASGFTFSRYSMNWVRQAPGKGLEWVSSISSSSTNINYADSVRGRFTISRDNAKNSLYLQMNSLRAEDTAVYYCAREYMWKVFDYWGQGTLVTVSSGTEGKSSGSGSESKSTDIVMTQTPLSLPVTPGEPASISCRSSQSLLDSDDGNIFLDWFLQKPGQSPQLLIYTVSYRASGVPDRFSGSGSGTDFTLKISRVEAEDVGVYYCMQRIEFPITFGQGTRLEIK

SEQ ID NO: 372 - protein, 218 aa, synthetic construct; identical to SEQ ID NO: 11
  REGION 1..218: note = BBBB467 LC

SEQ ID NO: 373 - protein, 449 aa, synthetic construct; identical to SEQ ID NO: 12
  REGION 1..449: note = BBBB467 HC2

SEQ ID NO: 374 - protein, 242 aa, synthetic construct
  REGION 1..242: note = TfR 21
EVQLVESGGGLVQPGGSLRLSCAASGFTFNSYDMHWVRQVTGKGLEWVSAIDTAGDTYYPGSVKGRFTISREDAKNSLYLQMNSLRAGDTAVYYCARDRLGYYGLDVWGQGTTVTVSSGTEGKSSGSGSESKSTEIVLTQSPGTLSLSPGERATLSCRASQSVSSSYLAWYQQKPGQAPRLLIYGASSRATGIPDRVSGSGSGTDFSLTISRLEPEDFAVYYCQQYDRSPITFGGGTKVEIK

SEQ ID NO: 375 - protein, 8 aa, synthetic construct
  REGION 1..8: note = TfR 21 HCDR1
GFTFNSYD

SEQ ID NO: 376 - protein, 7 aa, synthetic construct
  REGION 1..7: note = TfR 21 HCDR2
IDTAGDT

SEQ ID NO: 377 - protein, 12 aa, synthetic construct
  REGION 1..12: note = TfR 21 HCDR3
ARDRLGYYGLDV

SEQ ID NO: 378 - protein, 7 aa, synthetic construct
  REGION 1..7: note = TfR 21 LCDR1
QSVSSSY

SEQ ID NO: 379 - skipped

SEQ ID NO: 380 - protein, 9 aa, synthetic construct
  REGION 1..9: note = TfR 21 LCDR3
QQYDRSPIT

SEQ ID NO: 381 - protein, 697 aa, synthetic construct
  REGION 1..697: note = BBBB476 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVQPGGSLRLSCAASGFTFNSYDMHWVRQVTGKGLEWVSAIDTAGDTYYPGSVKGRFTISREDAKNSLYLQMNSLRAGDTAVYYCARDRLGYYGLDVWGQGTTVTVSSGTEGKSSGSGSESKSTEIVLTQSPGTLSLSPGERATLSCRASQSVSSSYLAWYQQKPGQAPRLLIYGASSRATGIPDRVSGSGSGTDFSLTISRLEPEDFAVYYCQQYDRSPITFGGGTKVEIK

SEQ ID NO: 382 - protein, 218 aa, synthetic construct; identical to SEQ ID NO: 11
  REGION 1..218: note = BBBB476 LC

SEQ ID NO: 383 - protein, 449 aa, synthetic construct; identical to SEQ ID NO: 12
  REGION 1..449: note = BBBB476 HC2

SEQ ID NO: 384 - protein, 248 aa, synthetic construct
  REGION 1..248: note = TfR 22
EVQVVESGGGLVKPGGSLRLSCAASGLTFNNHNMNWVRQAPGKGLEWVSYISSSSSYKYYADSVKGRFTISRDNAKNSLNLQMNSLRAEDTAVYYCARDGIAAFDAFDIWGQGTMVTVSSGTEGKSSGSGSESKSTDVVMTQSPLSLPVTLGHTASISCRSSQSLVYSDGITYLYWFQQRPGQSPRRLFYKVSNRDSGVPDRFSGSGSGTDFTLTISRVEAEDVGVYYCMQGTHWPPTFGQGTKVEIK

SEQ ID NO: 385 - protein, 8 aa, synthetic construct
  REGION 1..8: note = TfR 22 HCDR1
GLTFNNHN

SEQ ID NO: 386 - protein, 8 aa, synthetic construct
  REGION 1..8: note = TfR 22 HCDR2
ISSSSSYK

SEQ ID NO: 387 - protein, 12 aa, synthetic construct
  REGION 1..12: note = TfR 22 HCDR3
ARDGIAAFDAFD

SEQ ID NO: 388 - protein, 11 aa, synthetic construct
  REGION 1..11: note = TfR 22 LCDR1
QSLVYSDGITY

SEQ ID NO: 389 - skipped

SEQ ID NO: 390 - protein, 9 aa, synthetic construct
  REGION 1..9: note = TfR 22 LCDR3
MQGTHWPPT

SEQ ID NO: 391 - protein, 703 aa, synthetic construct
  REGION 1..703: note = BBBB478 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQVVESGGGLVKPGGSLRLSCAASGLTFNNHNMNWVRQAPGKGLEWVSYISSSSSYKYYADSVKGRFTISRDNAKNSLNLQMNSLRAEDTAVYYCARDGIAAFDAFDIWGQGTMVTVSSGTEGKSSGSGSESKSTDVVMTQSPLSLPVTLGHTASISCRSSQSLVYSDGITYLYWFQQRPGQSPRRLFYKVSNRDSGVPDRFSGSGSGTDFTLTISRVEAEDVGVYYCMQGTHWPPTFGQGTKVEIK

SEQ ID NO: 392 - protein, 218 aa, synthetic construct; identical to SEQ ID NO: 11
  REGION 1..218: note = BBBB478 LC

SEQ ID NO: 393 - protein, 449 aa, synthetic construct; identical to SEQ ID NO: 12
  REGION 1..449: note = BBBB478 HC2

SEQ ID NO: 394 - protein, 237 aa, synthetic construct
  REGION 1..237: note = TfR 23
QVTLRESGPALVKPTQTLTLTCTFSGFSLSTSGMSVSWVRQPPGKALEWLALIDWRDDKFYSTSLRTRLTISQDTSKNQVVLTMTNMDPVDTATYFCAGIPGYWGQGTLVTVSSGTEGKSSGSGSESKSTDIVMTQSQKFLSTSVGDRVSVTCKASQNVATNVVWYQRKPGQSPKALIYSASFRYSEVPDRFTGGGSGTDFTLTISNVQSEDLAEYVCQQYNNYPFTFGGGTKLEIK

SEQ ID NO: 395 - protein, 10 aa, synthetic construct
  REGION 1..10: note = TfR 23 HCDR1
GFSLSTSGMS

SEQ ID NO: 396 - protein, 7 aa, synthetic construct
  REGION 1..7: note = TfR 23 HCDR2
IDWRDDK

SEQ ID NO: 397 - protein, 6 aa, synthetic construct
  REGION 1..6: note = TfR 23 HCDR3
AGIPGY

SEQ ID NO: 398 - protein, 6 aa, synthetic construct
  REGION 1..6: note = TfR 23 LCDR1
QNVATN

SEQ ID NO: 399 - skipped

SEQ ID NO: 400 - protein, 9 aa, synthetic construct
  REGION 1..9: note = TfR 23 LCDR3
QQYNNYPFT

SEQ ID NO: 401 - protein, 692 aa, synthetic construct
  REGION 1..692: note = BBBB479 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVTLRESGPALVKPTQTLTLTCTFSGFSLSTSGMSVSWVRQPPGKALEWLALIDWRDDKFYSTSLRTRLTISQDTSKNQVVLTMTNMDPVDTATYFCAGIPGYWGQGTLVTVSSGTEGKSSGSGSESKSTDIVMTQSQKFLSTSVGDRVSVTCKASQNVATNVVWYQRKPGQSPKALIYSASFRYSEVPDRFTGGGSGTDFTLTISNVQSEDLAEYVCQQYNNYPFTFGGGTKLEIK

SEQ ID NO: 402 - protein, 218 aa, synthetic construct; identical to SEQ ID NO: 11
  REGION 1..218: note = BBBB479 LC

SEQ ID NO: 403 - protein, 449 aa, synthetic construct; identical to SEQ ID NO: 12
  REGION 1..449: note = BBBB479 HC2

SEQ ID NO: 404 - protein, 245 aa, synthetic construct
  REGION 1..245: note = TfR 24
QVLLVESGGGVVQPGRSLRLSCAASGFTFSTYDMHWVRQAPGKGLEWVAVIWYDGTNRYYADSVKGRFTISRDNSKNTLYLQMNSLRAEDTAVYYCARDHGYTKFSDAFDFWGQGTMVTVSSGTEGKSSGSGSESKSTDIQMTQSPSAMSASVGDSVTITCRASQGISNYLVWFQQKPGKDPKRLIYAASSLQSGVPSRFSGSGSGTEFTLTISSLQPEDFATYYCLQHNSYPLTFGGGTKVEIK

SEQ ID NO: 405 - protein, 8 aa, synthetic construct
  REGION 1..8: note = TfR 24 HCDR1
GFTFSTYD

SEQ ID NO: 406 - protein, 8 aa, synthetic construct
  REGION 1..8: note = TfR 24 HCDR2
IWYDGTNR

SEQ ID NO: 407 - protein, 15 aa, synthetic construct
  REGION 1..15: note = TfR 24 HCDR3
ARDHGYTKFSDAFDF

SEQ ID NO: 408 - protein, 6 aa, synthetic construct
  REGION 1..6: note = TfR 24 LCDR1
QGISNY

SEQ ID NO: 409 - skipped

SEQ ID NO: 410 - protein, 9 aa, synthetic construct
  REGION 1..9: note = TfR 24 LCDR3
LQHNSYPLT

SEQ ID NO: 411 - protein, 700 aa, synthetic construct
  REGION 1..700: note = BBBB482 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVLLVESGGGVVQPGRSLRLSCAASGFTFSTYDMHWVRQAPGKGLEWVAVIWYDGTNRYYADSVKGRFTISRDNSKNTLYLQMNSLRAEDTAVYYCARDHGYTKFSDAFDFWGQGTMVTVSSGTEGKSSGSGSESKSTDIQMTQSPSAMSASVGDSVTITCRASQGISNYLVWFQQKPGKDPKRLIYAASSLQSGVPSRFSGSGSGTEFTLTISSLQPEDFATYYCLQHNSYPLTFGGGTKVEIK

SEQ ID NO: 412 - protein, 218 aa, synthetic construct; identical to SEQ ID NO: 11
  REGION 1..218: note = BBBB482 LC

SEQ ID NO: 413 - protein, 449 aa, synthetic construct; identical to SEQ ID NO: 12
  REGION 1..449: note = BBBB482 HC2

SEQ ID NO: 414 - protein, 243 aa, synthetic construct
  REGION 1..243: note = TfR 25
QVQLQESGPGLVKPSETLSLTCTVSGGSISAYYWLWIRQPAGKGLEWLGRIYASGTTYYNPSLKSRITMSLDTSRNQFSLRLRSVTAADTAVYYCARQETDTTGYDYFDYWGQGTLVTVSSGTEGKSSGSGSESKSTSYELTQPPSVSMSPGQTASITCSGDKLGDKFVCWYQKQPGQSPVLVIYQDNKRPSGIPERFTGSNSGNTATLTISETQAMDEADYYCQTWDRSDAVFGGGTKLTVL

SEQ ID NO: 415 - protein, 8 aa, synthetic construct
  REGION 1..8: note = TfR 25 HCDR1
GGSISAYY

SEQ ID NO: 416 - protein, 7 aa, synthetic construct
  REGION 1..7: note = TfR 25 HCDR2
IYASGTT

SEQ ID NO: 417 - protein, 15 aa, synthetic construct
  REGION 1..15: note = TfR 25 HCDR3
ARQETDTTGYDYFDY

SEQ ID NO: 418 - protein, 6 aa, synthetic construct
  REGION 1..6: note = TfR 25 LCDR1
KLGDKF

SEQ ID NO: 419 - skipped

SEQ ID NO: 420 - protein, 9 aa, synthetic construct
  REGION 1..9: note = TfR 25 LCDR3
QTWDRSDAV

SEQ ID NO: 421 - protein, 698 aa, synthetic construct
  REGION 1..698: note = BBBB486 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLQESGPGLVKPSETLSLTCTVSGGSISAYYWLWIRQPAGKGLEWLGRIYASGTTYYNPSLKSRITMSLDTSRNQFSLRLRSVTAADTAVYYCARQETDTTGYDYFDYWGQGTLVTVSSGTEGKSSGSGSESKSTSYELTQPPSVSMSPGQTASITCSGDKLGDKFVCWYQKQPGQSPVLVIYQDNKRPSGIPERFTGSNSGNTATLTISETQAMDEADYYCQTWDRSDAVFGGGTKLTVL

SEQ ID NO: 422 - protein, 218 aa, synthetic construct; identical to SEQ ID NO: 11
  REGION 1..218: note = BBBB486 LC

SEQ ID NO: 423 - protein, 449 aa, synthetic construct; identical to SEQ ID NO: 12
  REGION 1..449: note = BBBB486 HC2

SEQ ID NO: 424 - protein, 246 aa, synthetic construct
  REGION 1..246: note = TfR 26
EVQLVESGGGLVQPGGSLRLSCVASGFTFSYYWMHWVRQAPGKGLVWVSRISSDGSSTTYADSVSGRFTISRDNAKNTLYLQMNSLRAEDTAVYYCAREQRWLKSYYYGMDVWGQGTTVTVSSGTEGKSSGSGSESKSTDIQLTQSPSFLSASVGDRVTITCRASQGINSYLGWYQQKQGKAPKLLIHAASTLQSGVPSRFSGSGSGTEFTFTISSLQPEDFASYYCQQLNSYPLTFGGGTKVENK

SEQ ID NO: 425 - protein, 8 aa, synthetic construct
  REGION 1..8: note = TfR 26 HCDR1
GFTFSYYW

SEQ ID NO: 426 - protein, 8 aa, synthetic construct
  REGION 1..8: note = TfR 26 HCDR2
ISSDGSST

SEQ ID NO: 427 - protein, 16 aa, synthetic construct
  REGION 1..16: note = TfR 26 HCDR3
AREQRWLKSYYYGMDV

SEQ ID NO: 428 - protein, 6 aa, synthetic construct
  REGION 1..6: note = TfR 26 LCDR1
QGINSY

SEQ ID NO: 429 - skipped

SEQ ID NO: 430 - protein, 9 aa, synthetic construct
  REGION 1..9: note = TfR 26 LCDR3
QQLNSYPLT

SEQ ID NO: 431 - protein, 701 aa, synthetic construct
  REGION 1..701: note = BBBB500 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVQPGGSLRLSCVASGFTFSYYWMHWVRQAPGKGLVWVSRISSDGSSTTYADSVSGRFTISRDNAKNTLYLQMNSLRAEDTAVYYCAREQRWLKSYYYGMDVWGQGTTVTVSSGTEGKSSGSGSESKSTDIQLTQSPSFLSASVGDRVTITCRASQGINSYLGWYQQKQGKAPKLLIHAASTLQSGVPSRFSGSGSGTEFTFTISSLQPEDFASYYCQQLNSYPLTFGGGTKVENK

SEQ ID NO: 432 - protein, 218 aa, synthetic construct; identical to SEQ ID NO: 11
  REGION 1..218: note = BBBB500 LC

SEQ ID NO: 433 - protein, 449 aa, synthetic construct; identical to SEQ ID NO: 12
  REGION 1..449: note = BBBB500 HC2

SEQ ID NO: 434 - protein, 243 aa, synthetic construct
  REGION 1..243: note = TfR 27
DVQLVESGGGLVKPGGSLRLSCAASAFRFSNFNMNWVRQAPGKGLEWVSSITSTGTYIYYADSLEGRFTISRDNAKNSLFLQMNSLRAEDTAVYYCARQGIPAWDAFDLWGQGTMVTVSSGTEGKSSGSGSESKSTDIQMTQSPSAMSASVGDRVTITCRASQGISNYLAWFQQKPGKVPKRLIYAASSLQSGVPSRFSGSGSGTEFTLTISSLQPEDFATYYCLQHNSYPYTFGQGTKLEIK

SEQ ID NO: 435 - protein, 8 aa, synthetic construct
  REGION 1..8: note = TfR 27 HCDR1
AFRFSNFN

SEQ ID NO: 436 - protein, 8 aa, synthetic construct
  REGION 1..8: note = TfR 27 HCDR2
ITSTGTYI

SEQ ID NO: 437 - protein, 13 aa, synthetic construct
  REGION 1..13: note = TfR 27 HCDR3
ARQGIPAWDAFDL

SEQ ID NO: 438 - protein, 6 aa, synthetic construct
  REGION 1..6: note = TfR 27 LCDR1
QGISNY

SEQ ID NO: 439 - skipped

SEQ ID NO: 440 - protein, 9 aa, synthetic construct
  REGION 1..9: note = TfR 27 LCDR3
LQHNSYPYT

SEQ ID NO: 441 - protein, 698 aa, synthetic construct
  REGION 1..698: note = BBBB504 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSDVQLVESGGGLVKPGGSLRLSCAASAFRFSNFNMNWVRQAPGKGLEWVSSITSTGTYIYYADSLEGRFTISRDNAKNSLFLQMNSLRAEDTAVYYCARQGIPAWDAFDLWGQGTMVTVSSGTEGKSSGSGSESKSTDIQMTQSPSAMSASVGDRVTITCRASQGISNYLAWFQQKPGKVPKRLIYAASSLQSGVPSRFSGSGSGTEFTLTISSLQPEDFATYYCLQHNSYPYTFGQGTKLEIK

SEQ ID NO: 442 - protein, 218 aa, synthetic construct; identical to SEQ ID NO: 11
  REGION 1..218: note = BBBB504 LC

SEQ ID NO: 443 - protein, 449 aa, synthetic construct; identical to SEQ ID NO: 12
  REGION 1..449: note = BBBB504 HC2

SEQ ID NO: 444 - protein, 243 aa, synthetic construct
  REGION 1..243: note = TfR 28
EVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTDIQMTQSPSAMSASVGDRVTITCRASQGISNFFAWFQQKPGKVPKRLIYAASSLQSGVPSRFSGSGSGTEFTLTISSLQPEDFATYYCLQHDSYPLTFGGGTKVEIK

SEQ ID NO: 445 - protein, 8 aa, synthetic construct
  REGION 1..8: note = TfR 28 HCDR1
GFTFSSYN

SEQ ID NO: 446 - protein, 8 aa, synthetic construct
  REGION 1..8: note = TfR 28 HCDR2
ISSSSSYI

SEQ ID NO: 447 - protein, 13 aa, synthetic construct
  REGION 1..13: note = TfR 28 HCDR3
AREGISAYDALNV

SEQ ID NO: 448 - protein, 6 aa, synthetic construct
  REGION 1..6: note = TfR 28 LCDR1
QGISNF

SEQ ID NO: 449 - skipped

SEQ ID NO: 450 - protein, 9 aa, synthetic construct
  REGION 1..9: note = TfR 28 LCDR3
LQHDSYPLT

SEQ ID NO: 451 - protein, 698 aa, synthetic construct
  REGION 1..698: note = BBBB508 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLVESGGGLVKPGGSLRLSCVASGFTFSSYNMNWVRQAPGKGLEWVSSISSSSSYIYYADSVKGRFTISRDNAKNSLYLQMNSLRAEDTAVYYCAREGISAYDALNVWGQGTMVTVSSGTEGKSSGSGSESKSTDIQMTQSPSAMSASVGDRVTITCRASQGISNFFAWFQQKPGKVPKRLIYAASSLQSGVPSRFSGSGSGTEFTLTISSLQPEDFATYYCLQHDSYPLTFGGGTKVEIK

SEQ ID NO: 452 - protein, 218 aa, synthetic construct; identical to SEQ ID NO: 11
  REGION 1..218: note = BBBB508 LC

SEQ ID NO: 453 - protein, 449 aa, synthetic construct; identical to SEQ ID NO: 12
  REGION 1..449: note = BBBB508 HC2

SEQ ID NO: 454 - protein, 246 aa, synthetic construct; identical to SEQ ID NO: 56
  REGION 1..246: note = TfR 29

SEQ ID NO: 455 - protein, 8 aa, synthetic construct
  REGION 1..8: note = TfR 29 HCDR1
GFTFSSYN

SEQ ID NO: 456 - protein, 8 aa, synthetic construct
  REGION 1..8: note = TfR 29 HCDR2
ISSSSSYI

SEQ ID NO: 457 - protein, 13 aa, synthetic construct
  REGION 1..13: note = TfR 29 HCDR3
AREGISAYDALNV

SEQ ID NO: 458 - protein, 8 aa, synthetic construct
  REGION 1..8: note = TfR 29 LCDR1
SSNIGNNY

SEQ ID NO: 459 - skipped

SEQ ID NO: 460 - protein, 11 aa, synthetic construct
  REGION 1..11: note = TfR 29 LCDR3
GTWDSSLSAVV

SEQ ID NO: 461 - protein, 701 aa, synthetic construct; identical to SEQ ID NO: 63
  REGION 1..701: note = BBBB509 HC1

SEQ ID NO: 462 - protein, 218 aa, synthetic construct; identical to SEQ ID NO: 11
  REGION 1..218: note = BBBB509 LC

SEQ ID NO: 463 - protein, 449 aa, synthetic construct; identical to SEQ ID NO: 12
  REGION 1..449: note = BBBB509 HC2

SEQ ID NO: 464 - protein, 245 aa, synthetic construct
  REGION 1..245: note = TfR 30
QVQLVESGGGLVKPGGSLRLSCAASGFTFSDYYMSWIRQAPGKGLECISYISSSSVTIYYADSVKGRFTISRDNAKNSLYLQMNSLRAEDTAVYYCAREDSDTSGYDPFDHWGQGTLVTVSSGTEGKSSGSGSESKSTEIVMTQSPATLSVSPGERATLSCRASQSVSSNLAWYQQKPGQAPRLLIYGASTRATGIPARFSGSGSGTEFTLTISSLQSEDFAVYYCQQYNNWPYTFGQGTKLEIK

SEQ ID NO: 465 - protein, 8 aa, synthetic construct
  REGION 1..8: note = TfR 30 HCDR1
GFTFSDYY

SEQ ID NO: 466 - protein, 8 aa, synthetic construct
  REGION 1..8: note = TfR 30 HCDR2
ISSSSVTI

SEQ ID NO: 467 - protein, 15 aa, synthetic construct
  REGION 1..15: note = TfR 30 HCDR3
AREDSDTSGYDPFDH

SEQ ID NO: 468 - protein, 6 aa, synthetic construct
  REGION 1..6: note = TfR 30 LCDR1
QSVSSN

SEQ ID NO: 469 - skipped

SEQ ID NO: 470 - protein, 9 aa, synthetic construct
  REGION 1..9: note = TfR 30 LCDR3
QQYNNWPYT

SEQ ID NO: 471 - protein, 700 aa, synthetic construct
  REGION 1..700: note = BBBB522 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVKPGGSLRLSCAASGFTFSDYYMSWIRQAPGKGLECISYISSSSVTIYYADSVKGRFTISRDNAKNSLYLQMNSLRAEDTAVYYCAREDSDTSGYDPFDHWGQGTLVTVSSGTEGKSSGSGSESKSTEIVMTQSPATLSVSPGERATLSCRASQSVSSNLAWYQQKPGQAPRLLIYGASTRATGIPARFSGSGSGTEFTLTISSLQSEDFAVYYCQQYNNWPYTFGQGTKLEIK

SEQ ID NO: 472 - protein, 218 aa, synthetic construct; identical to SEQ ID NO: 11
  REGION 1..218: note = BBBB522 LC

SEQ ID NO: 473 - protein, 449 aa, synthetic construct; identical to SEQ ID NO: 12
  REGION 1..449: note = BBBB522 HC2

SEQ ID NO: 474 - protein, 244 aa, synthetic construct
  REGION 1..244: note = TfR 31
QVQLVESGGGLVKPGGSLRLSCEASGFTLSDYDMTWIRQAPGKGLEWVSYINSGGNIISYADSVKGRFTISRDNAKNSLYLQMNSLRAEDTAVFYCARLYYDTSGYSSFDYWGQGIQVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 475 - protein, 8 aa, synthetic construct
  REGION 1..8: note = TfR 31 HCDR1
GFTLSDYD

SEQ ID NO: 476 - protein, 8 aa, synthetic construct
  REGION 1..8: note = TfR 31 HCDR2
INSGGNII

SEQ ID NO: 477 - protein, 15 aa, synthetic construct
  REGION 1..15: note = TfR 31 HCDR3
ARLYYDTSGYSSFDY

SEQ ID NO: 478 - protein, 6 aa, synthetic construct
  REGION 1..6: note = TfR 31 LCDR1
KLGDKF

SEQ ID NO: 479 - skipped

SEQ ID NO: 480 - protein, 9 aa, synthetic construct
  REGION 1..9: note = TfR 31 LCDR3
QTWYSSTVI

SEQ ID NO: 481 - protein, 699 aa, synthetic construct
  REGION 1..699: note = BBBB529 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGLVKPGGSLRLSCEASGFTLSDYDMTWIRQAPGKGLEWVSYINSGGNIISYADSVKGRFTISRDNAKNSLYLQMNSLRAEDTAVFYCARLYYDTSGYSSFDYWGQGIQVTVSSGTEGKSSGSGSESKSTSYEVTQPPSVSVSPGQTASITCSGHKLGDKFACWYQQKPGQSPVLVIYQDRKRPSGIPERFSGSNSGNTATLTISGTQAMDEADYYCQTWYSSTVIFGGGTKLTVL

SEQ ID NO: 482 - protein, 218 aa, synthetic construct; identical to SEQ ID NO: 11
  REGION 1..218: note = BBBB529 LC

SEQ ID NO: 483 - protein, 449 aa, synthetic construct; identical to SEQ ID NO: 12
  REGION 1..449: note = BBBB529 HC2

SEQ ID NO: 484 - protein, 244 aa, synthetic construct
  REGION 1..244: note = TfR 32
EVQLLESGGGLVQPGGSLRLSCAASGFTFSSYAMSWVRQAPGKGLEWVSGISAGGGSTYYADSVKGRFTVSRGNSKNTLYLQMNSLRAEDTAVYFCAKREDDTTGYHYFDYWGQGTLVAVSSGTEGKSSGSGSESKSTSYELTQAPSVSVSPGQTASITCSGDKLGDKFTCWYQQKPGHSPVLVIYQDTKRPSGIPERFSGSNSGNTATLTISGIQAMDEADYYCQAWDRTTVVFGGGTKLTVL

SEQ ID NO: 485 - protein, 8 aa, synthetic construct
  REGION 1..8: note = TfR 32 HCDR1
GFTFSSYA

SEQ ID NO: 486 - protein, 8 aa, synthetic construct
  REGION 1..8: note = TfR 32 HCDR2
ISAGGGST

SEQ ID NO: 487 - protein, 15 aa, synthetic construct
  REGION 1..15: note = TfR 32 HCDR3
AKREDDTTGYHYFDY

SEQ ID NO: 488 - protein, 6 aa, synthetic construct
  REGION 1..6: note = TfR 32 LCDR1
KLGDKF

SEQ ID NO: 489 - skipped

SEQ ID NO: 490 - protein, 9 aa, synthetic construct
  REGION 1..9: note = TfR 32 LCDR3
QAWDRTTVV

SEQ ID NO: 491 - protein, 699 aa, synthetic construct
  REGION 1..699: note = BBBB530 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCAASGFTFSSYAMSWVRQAPGKGLEWVSGISAGGGSTYYADSVKGRFTVSRGNSKNTLYLQMNSLRAEDTAVYFCAKREDDTTGYHYFDYWGQGTLVAVSSGTEGKSSGSGSESKSTSYELTQAPSVSVSPGQTASITCSGDKLGDKFTCWYQQKPGHSPVLVIYQDTKRPSGIPERFSGSNSGNTATLTISGIQAMDEADYYCQAWDRTTVVFGGGTKLTVL

SEQ ID NO: 492 - protein, 218 aa, synthetic construct; identical to SEQ ID NO: 11
  REGION 1..218: note = BBBB530 LC

SEQ ID NO: 493 - protein, 449 aa, synthetic construct; identical to SEQ ID NO: 12
  REGION 1..449: note = BBBB530 HC2

SEQ ID NO: 494 - protein, 246 aa, synthetic construct
  REGION 1..246: note = TfR 33
QVQLQESGPGLVKPSDTLSLTCAVSGYSISSSNWWGWIRQPPGKGLEWIGYIYFSGSTYFNPSLKSRVTMSVDTSKNQFSLRLSSVTAVDTAVYYCARKKGAYDYADAFDIWGQGTMVTVSSGTEGKSSGSGSESKSTSYVVTQPPSVSVAPGQTARITCGGNKIGSKSVHWFQQKPGQAPVLVVYDDSDRPSGIPERFSGSNSGNTATLTISRVEAGDEADYYCQVWDSSSDHVVFGGGTKLIVL

SEQ ID NO: 495 - protein, 10 aa, synthetic construct
  REGION 1..10: note = TfR 33 HCDR1
GYSISSSNWW

SEQ ID NO: 496 - protein, 7 aa, synthetic construct
  REGION 1..7: note = TfR 33 HCDR2
IYFSGST

SEQ ID NO: 497 - protein, 15 aa, synthetic construct
  REGION 1..15: note = TfR 33 HCDR3
ARKKGAYDYADAFDI

SEQ ID NO: 498 - protein, 6 aa, synthetic construct
  REGION 1..6: note = TfR 33 LCDR1
KIGSKS

SEQ ID NO: 499 - skipped

SEQ ID NO: 500 - protein, 11 aa, synthetic construct
  REGION 1..11: note = TfR 33 LCDR3
QVWDSSSDHVV

SEQ ID NO: 501 - protein, 701 aa, synthetic construct
  REGION 1..701: note = BBBB532 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLQESGPGLVKPSDTLSLTCAVSGYSISSSNWWGWIRQPPGKGLEWIGYIYFSGSTYFNPSLKSRVTMSVDTSKNQFSLRLSSVTAVDTAVYYCARKKGAYDYADAFDIWGQGTMVTVSSGTEGKSSGSGSESKSTSYVVTQPPSVSVAPGQTARITCGGNKIGSKSVHWFQQKPGQAPVLVVYDDSDRPSGIPERFSGSNSGNTATLTISRVEAGDEADYYCQVWDSSSDHVVFGGGTKLIVL

SEQ ID NO: 502 - protein, 218 aa, synthetic construct; identical to SEQ ID NO: 11
  REGION 1..218: note = BBBB532 LC

SEQ ID NO: 503 - protein, 449 aa, synthetic construct; identical to SEQ ID NO: 12
  REGION 1..449: note = BBBB532 HC2

SEQ ID NO: 504 - protein, 242 aa, synthetic construct
  REGION 1..242: note = TfR 34
QLQVQESGPGLVKPSETLSLTCTVSGDSITNSNFYWGWIRQPPGKGLEWIGSIFHGGNTYYNPSLKSRVTISVDTSKNQFSLRLRSVTAADTAVYYCARYVAAPDYFDYWGQGSLVTVSSGTEGKSSGSGSESKSTSYELTQPPSVSVSPGKTASITCSGDNLGNKFACWYLQKAGQSPVLVIYQDRNRPSGIPERFSGSNSGNTATLTISGTQAMDEGDYYCQAWDSSTVVFGGGTKLTVL

SEQ ID NO: 505 - protein, 10 aa, synthetic construct
  REGION 1..10: note = TfR 34 HCDR1
GDSITNSNFY

SEQ ID NO: 506 - protein, 7 aa, synthetic construct
  REGION 1..7: note = TfR 34 HCDR2
IFHGGNT

SEQ ID NO: 507 - protein, 12 aa, synthetic construct
  REGION 1..12: note = TfR 34 HCDR3
ARYVAAPDYFDY

SEQ ID NO: 508 - protein, 6 aa, synthetic construct
  REGION 1..6: note = TfR 34 LCDR1
NLGNKF

SEQ ID NO: 509 - skipped

SEQ ID NO: 510 - protein, 9 aa, synthetic construct
  REGION 1..9: note = TfR 34 LCDR3
QAWDSSTVV

SEQ ID NO: 511 - protein, 697 aa, synthetic construct
  REGION 1..697: note = BBBB538 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQLQVQESGPGLVKPSETLSLTCTVSGDSITNSNFYWGWIRQPPGKGLEWIGSIFHGGNTYYNPSLKSRVTISVDTSKNQFSLRLRSVTAADTAVYYCARYVAAPDYFDYWGQGSLVTVSSGTEGKSSGSGSESKSTSYELTQPPSVSVSPGKTASITCSGDNLGNKFACWYLQKAGQSPVLVIYQDRNRPSGIPERFSGSNSGNTATLTISGTQAMDEGDYYCQAWDSSTVVFGGGTKLTVL

SEQ ID NO: 512 - protein, 218 aa, synthetic construct; identical to SEQ ID NO: 11
  REGION 1..218: note = BBBB538 LC

SEQ ID NO: 513 - protein, 449 aa, synthetic construct; identical to SEQ ID NO: 12
  REGION 1..449: note = BBBB538 HC2

SEQ ID NO: 514 - protein, 244 aa, synthetic construct
  REGION 1..244: note = TfR 35
EVQLLESGGGLVQPGGSLRLSCAASGFTFSSYVMTWARQAPGKGLEWVSTISGSGGNTYYADSVKGRFTISRDNPTNTLFLQMNSLRPEDTALYYCAKLDYDTSGYDPFDFWGQGTMVTVSSGTEGKSSGSGSESKSTSYGLTQPPSVSVSPGQTASITCSGHKLGDKFACWYQQKPGQSPVLVIYQDNKRPSGIPERFSGSNSGNTATLTISGTQAMDEADYYCQAWVSSTAIFGGGTKLTVL

SEQ ID NO: 515 - protein, 8 aa, synthetic construct
  REGION 1..8: note = TfR 35 HCDR1
GFTFSSYV

SEQ ID NO: 516 - protein, 8 aa, synthetic construct
  REGION 1..8: note = TfR 35 HCDR2
ISGSGGNT

SEQ ID NO: 517 - protein, 15 aa, synthetic construct
  REGION 1..15: note = TfR 35 HCDR3
AKLDYDTSGYDPFDF

SEQ ID NO: 518 - protein, 6 aa, synthetic construct
  REGION 1..6: note = TfR 35 LCDR1
KLGDKF

SEQ ID NO: 519 - skipped

SEQ ID NO: 520 - protein, 9 aa, synthetic construct
  REGION 1..9: note = TfR 35 LCDR3
QAWVSSTAI

SEQ ID NO: 521 - protein, 699 aa, synthetic construct
  REGION 1..699: note = BBBB539 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSEVQLLESGGGLVQPGGSLRLSCAASGFTFSSYVMTWARQAPGKGLEWVSTISGSGGNTYYADSVKGRFTISRDNPTNTLFLQMNSLRPEDTALYYCAKLDYDTSGYDPFDFWGQGTMVTVSSGTEGKSSGSGSESKSTSYGLTQPPSVSVSPGQTASITCSGHKLGDKFACWYQQKPGQSPVLVIYQDNKRPSGIPERFSGSNSGNTATLTISGTQAMDEADYYCQAWVSSTAIFGGGTKLTVL

SEQ ID NO: 522 - protein, 218 aa, synthetic construct; identical to SEQ ID NO: 11
  REGION 1..218: note = BBBB539 LC

SEQ ID NO: 523 - protein, 449 aa, synthetic construct; identical to SEQ ID NO: 12
  REGION 1..449: note = BBBB539 HC2

SEQ ID NO: 524 - protein, 246 aa, synthetic construct
  REGION 1..246: note = TfR 36
QVQLVESGGGVVQPGRSLRLSCAASGFTFSSDGMHWVRQAPGKGLEWVAVIWYDGSNKYYADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYTDSNNFDVLFGGGTKLTVL

SEQ ID NO: 525 - protein, 8 aa, synthetic construct
  REGION 1..8: note = TfR 36 HCDR1
GFTFSSDG

SEQ ID NO: 526 - protein, 8 aa, synthetic construct
  REGION 1..8: note = TfR 36 HCDR2
IWYDGSNK

SEQ ID NO: 527 - protein, 11 aa, synthetic construct
  REGION 1..11: note = TfR 36 HCDR3
ARDRQWLAFDY

SEQ ID NO: 528 - protein, 9 aa, synthetic construct
  REGION 1..9: note = TfR 36 LCDR1
SSDVGGYNF

SEQ ID NO: 529 - skipped

SEQ ID NO: 530 - protein, 12 aa, synthetic construct
  REGION 1..12: note = TfR 36 LCDR3
SSYTDSNNFDVL

SEQ ID NO: 531 - protein, 701 aa, synthetic construct
  REGION 1..701: note = BBBB540 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVESGGGVVQPGRSLRLSCAASGFTFSSDGMHWVRQAPGKGLEWVAVIWYDGSNKYYADSVKGRFTISRDNSKNTLYLQMNSLRAEDTAVYYCARDRQWLAFDYWGQGTLVTVSSGTEGKSSGSGSESKSTQSALTQPPSASGSPGQSVTISCSGTSSDVGGYNFVSWYQQHPGKAPKLMISEVSKRPSGVPDRFSGSKSGNTASLTVSGLQAEDEADYYCSSYTDSNNFDVLFGGGTKLTVL

SEQ ID NO: 532 - protein, 218 aa, synthetic construct; identical to SEQ ID NO: 11
  REGION 1..218: note = BBBB540 LC

SEQ ID NO: 533 - protein, 449 aa, synthetic construct; identical to SEQ ID NO: 12
  REGION 1..449: note = BBBB540 HC2

SEQ ID NO: 534 - protein, 244 aa, synthetic construct
  REGION 1..244: note = TfR 37
QVQLVQSGAEVKKPGASVKVSCKASGYTFTSYDIHWVRQATGQGLEWMGWMNPNSGDTGYAQKFQGRVSMTRDTSMSTAYMELSRLRSGDTAVYYCMKMYYDTTGYHSFDSWGQGTLVTVSSGTEGKSSGSGSESKSTSYDLTQPPSVSVSPGQTANITCSGDRLGDRFGCWYQQKPGQSPVLVIYQDTKRPSGIPERFSGSNSGNTATLTISGTQAMDEADYYCQTWVATTVVFGGGTKLTVL

SEQ ID NO: 535 - protein, 8 aa, synthetic construct
  REGION 1..8: note = TfR 37 HCDR1
GYTFTSYD

SEQ ID NO: 536 - protein, 8 aa, synthetic construct
  REGION 1..8: note = TfR 37 HCDR2
MNPNSGDT

SEQ ID NO: 537 - protein, 15 aa, synthetic construct
  REGION 1..15: note = TfR 37 HCDR3
MKMYYDTTGYHSFDS

SEQ ID NO: 538 - protein, 6 aa, synthetic construct
  REGION 1..6: note = TfR 37 LCDR1
RLGDRF

SEQ ID NO: 539 - skipped

SEQ ID NO: 540 - protein, 9 aa, synthetic construct
  REGION 1..9: note = TfR 37 LCDR3
QTWVATTVV

SEQ ID NO: 541 - protein, 699 aa, synthetic construct
  REGION 1..699: note = BBBB546 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQVQLVQSGAEVKKPGASVKVSCKASGYTFTSYDIHWVRQATGQGLEWMGWMNPNSGDTGYAQKFQGRVSMTRDTSMSTAYMELSRLRSGDTAVYYCMKMYYDTTGYHSFDSWGQGTLVTVSSGTEGKSSGSGSESKSTSYDLTQPPSVSVSPGQTANITCSGDRLGDRFGCWYQQKPGQSPVLVIYQDTKRPSGIPERFSGSNSGNTATLTISGTQAMDEADYYCQTWVATTVVFGGGTKLTVL

SEQ ID NO: 542 - protein, 218 aa, synthetic construct; identical to SEQ ID NO: 11
  REGION 1..218: note = BBBB546 LC

SEQ ID NO: 543 - protein, 449 aa, synthetic construct; identical to SEQ ID NO: 12
  REGION 1..449: note = BBBB546 HC2

SEQ ID NO: 544 - protein, 242 aa, synthetic construct
  REGION 1..242: note = TfR 38
QLQVQESGPGLVKPSETLSLTCTVSGGSISNSNYYWGLIRQPPGKGLEWIGSIFHGGNTYYNPSLKSRVTISVDTSKNQFSLRLRSVTAADTAVYYCARYVAAPDYFDYWGQGTLVTVSSGTEGKSSGSGSESKSTSYELTQPPSVSVSPGKTASITCSGDKLGDKFACWYQQKAGQSPVLVIYQDRKRPSGIPERFSGSNSGNTATLTISGTQTMDEGDYYCQAWDSSTVVFGGGTKLTVL

SEQ ID NO: 545 - protein, 10 aa, synthetic construct
  REGION 1..10: note = TfR 38 HCDR1
GGSISNSNYY

SEQ ID NO: 546 - protein, 7 aa, synthetic construct
  REGION 1..7: note = TfR 38 HCDR2
IFHGGNT

SEQ ID NO: 547 - protein, 12 aa, synthetic construct
  REGION 1..12: note = TfR 38 HCDR3
ARYVAAPDYFDY

SEQ ID NO: 548 - protein, 6 aa, synthetic construct
  REGION 1..6: note = TfR 38 LCDR1
KLGDKF

SEQ ID NO: 549 - skipped

SEQ ID NO: 550 - protein, 9 aa, synthetic construct
  REGION 1..9: note = TfR 38 LCDR3
QAWDSSTVV

SEQ ID NO: 551 - protein, 697 aa, synthetic construct
  REGION 1..697: note = BBBB547 HC1
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGGGSGGSQLQVQESGPGLVKPSETLSLTCTVSGGSISNSNYYWGLIRQPPGKGLEWIGSIFHGGNTYYNPSLKSRVTISVDTSKNQFSLRLRSVTAADTAVYYCARYVAAPDYFDYWGQGTLVTVSSGTEGKSSGSGSESKSTSYELTQPPSVSVSPGKTASITCSGDKLGDKFACWYQQKAGQSPVLVIYQDRKRPSGIPERFSGSNSGNTATLTISGTQTMDEGDYYCQAWDSSTVVFGGGTKLTVL

SEQ ID NO: 552 - protein, 218 aa, synthetic construct; identical to SEQ ID NO: 11
  REGION 1..218: note = BBBB547 LC

SEQ ID NO: 553 - protein, 449 aa, synthetic construct; identical to SEQ ID NO: 12
  REGION 1..449: note = BBBB547 HC2

SEQ ID NO: 554 - protein, 10 aa, synthetic construct
  REGION 1..10: note = PT1B844 HCDR1
GFTFSSYAMS

SEQ ID NO: 555 - protein, 9 aa, synthetic construct
  REGION 1..9: note = PT1B844 HCDR2
SISKGGNTY

SEQ ID NO: 556 - protein, 10 aa, synthetic construct
  REGION 1..10: note = PT1B844 HCDR3
GWGDYGWFAY

SEQ ID NO: 557 - protein, 11 aa, synthetic construct
  REGION 1..11: note = PT1B844 LCDR1
KASQDINRYLN

SEQ ID NO: 558 - protein, 7 aa, synthetic construct
  REGION 1..7: note = PT1B844 LCDR2
RANRLLD

SEQ ID NO: 559 - protein, 9 aa, synthetic construct
  REGION 1..9: note = PT1B844 LCDR3
QQYDEFPLT

(19) United States
(12) Patent Application Publication
(10) Pub. No.: US 2026/0008846 A1
(43) Pub. Date: Jan. 8, 2026

(54) COMPOSITIONS AND METHODS FOR BLOOD-BRAIN BARRIER DELIVERY

(71) Applicant: Aliada Therapeutics, Inc., Boston, MA (US)

(72) Inventors: Suzanne EDAVETTAL, San Diego, CA (US); Sanjaya Singh, Blue Bell, PA (US); Derrick Domingo, San Diego, CA (US); Deepti Wilkinson, Del Mar, CA (US); Pilar Cejudo-Martin, San Diego, CA (US); Pharavee Jaiprasart, Spring House, PA (US); Brian Geist, Spring House, PA (US)

(21) Appl. No.: 19/334,516

(22) Filed: Sep. 19, 2025

Related U.S. Application Data
(62) Division of application No. 17/995,768, filed on Oct. 7, 2022.
(60) Provisional application No. 63/006,998, filed on Apr. 8, 2020.
(60) Provisional application No. 63/036,020, filed on Jun. 8, 2020.

(51) Int. Cl. C07K 16/28; A61K 9/00; A61K 39/395; A61K 47/68; C12N 15/62; C12N 15/63

(52) U.S. Cl. CPC C07K 16/28; A61K 9/0019; A61K 39/39533; A61K 47/6849; C12N 15/62; C12N 15/63; C07K 2317/31; C07K 2317/622; C07K 2317/92; C12N 2320/32

(57) ABSTRACT

Monoclonal anti-TfR antibodies and antigen-binding fragments thereof for delivering an agent to the brain of a subject in need thereof are described. Also described are conjugates and fusion constructs containing the anti-TfR antibody or antigen-binding fragment thereof coupled to a therapeutic or diagnostic agent, such as a second antibody and antigen-binding fragment thereof, for treating or detecting a neurological disorder and/or delivering a therapeutic or diagnostic agent across the blood-brain barrier. Also described are nucleic acids encoding the antibodies, conjugates and fusion constructs and related recombinant host cells.

### CROSS REFERENCE TO RELATED APPLICATIONS

The present application is a division of U.S. patent application Ser. No. 17/995,768, filed on Oct. 7, 2022, which is a U.S. National Stage Entry of International Patent Application No. PCT/IB2021/052889, filed on Apr. 7, 2021, which claims priority to U.S. Provisional Application No. 63/006,998, filed on Apr. 8, 2020, and U.S. Provisional Application No. 63/036,020, filed on Jun. 8, 2020, the disclosures of which are incorporated herein by reference in their entireties.

### REFERENCE TO SEQUENCE LISTING SUBMITTED ELECTRONICALLY

The instant application contains a Sequence Listing, which has been submitted electronically in XML format and is hereby incorporated by reference in its entirety. Said XML copy, created on Sep. 4, 2025, is named “51873-004004_Sequence_Listing_9_4_25” and is 810,683 bytes in size.

### FIELD OF THE INVENTION

The present invention relates to a blood-brain barrier shuttle that binds to the transferrin receptor (TfR) and methods of using the same.

### BACKGROUND OF THE INVENTION

While the blood-brain barrier (BBB) prevents harmful substances from entering the brain and is essential for brain homeostasis, it presents a formidable obstacle for efficiently delivering drugs to the brain. Large molecules, such as monoclonal antibodies and other biotherapeutics, have great therapeutic/diagnostic potential for treating/detecting pathology in the central nervous system (CNS). However, their route into the brain is prevented by the BBB. Previous studies have illustrated that only a very small percentage (approximately 0.1%) of an IgG injected in the bloodstream are able to penetrate the BBB into the CNS compartment (Felgenhauer, Klin. Wschr. 52: 1158-1164, 1974)). This will limit any pharmacological effect due to the low concentration within the CNS of the antibody.

Numerous approaches have been studied to improve the brain delivery of therapeutic monoclonal antibodies (mAbs), including the use of receptor-mediated transcytosis (RMT). RMT utilizes abundantly expressed receptors on the luminal side of the BBB for transport through brain endothelial cells. Previous efforts to generate a clinically feasible platform for delivery of therapeutic mAbs into the brain have been focused on antibody engineering to increase the efficiency of transcytosis, with gains made through observations on valency of binding, pH dependency and affinity (reviewed in Goulatis et al., 2017, Curr Opin Struct Biol 45: 109-115). However, translation into NHPs and the clinic has been limited by rapid peripheral clearance from target-mediated drug disposition (TMDD) and safety from acute reticulocyte depletion (Gadkar, 2016, Eur J Pharm Biopharm. 2016 April; 101:53-61). Transferrin receptor (TfR), particularly TfR1, mediates the transport of iron-loaded transferrin (Tf) from blood to brain and the return of iron-depleted Tf to the blood (Kawabata, Free Radical Biology & Medicine, 133, 46-54, 2019). Anti-TfR1 monoclonal antibodies have been used to deliver drugs to the brain (Burkhart, et al. Progress in neurobiology, 181, 101665, 2019). However, safety liabilities and poor pharmacokinetics (PK) of anti-TfR1 monoclonal antibodies have hampered their clinical development as BBB carriers.

Therefore, there is a need for an anti-TfR monoclonal antibody or antigen binding fragment thereof that can be used to shuttle drugs into the brain efficiently with improved safety and PK.

### SUMMARY OF THE INVENTION

The application relates to an optimized platform for brain delivery, accounting for not just brain concentration of a delivered agent, such as a therapeutic monoclonal antibody (mAb), but also therapeutically relevant characteristics of the mAb, including peripheral pharmacokinetics, safety and the pharmacodynamics of the mAb. The platform utilizes a TfR binding molecule, in particular, an antibody or antigen-binding fragment thereof that binds to transferrin receptor (TfR), preferably a human transferrin receptor 1 (huTfR1), wherein the TfR binding molecule has optimized transport function defined by the on-rate kₐ and off-rate k_{d} values both at a neutral pH of 6.8 to 7.8, such as a physiological pH (e.g., 7.4), and at an acidic pH of 4.5 to 6.5, such as an acidic pH often found in endosomal compartments.

The inventors discovered, unexpectedly, the optimal values are not simply the fastest on-rate kₐ values and the slowest off-rate k_{d} values as one might expect in typical antibody-target interactions. That is, for this system, one would not necessarily want to use a molecule that “binds” and associates with TfR at a relatively high rate and then dissociates from the TfR more slowly to have the longest life span of the antibody-target complex. Instead, in one embodiment, the optimized transport function of the TfR binders described herein preferably have kₐ rates that are similar (e.g., within the same order of magnitude) at both physiologic pH (e.g., 7.4) and at lower pH (e.g., 6.5 or 6.0) but have faster off-rate k_{d} at a lower pH (e.g., pH 6.5 or 6.0) when compared to the k_{d} rates at physiological pH (e.g., 7.4).

In one general aspect, the application describes an anti-TfR antibody or antigen-binding fragment thereof for delivering a therapeutic or diagnostic agent to the brain of a subject in need thereof, wherein the anti-TfR antibody or antigen-binding fragment thereof binds to a transferrin receptor (TfR), preferably human TfR1, with a dissociation constant K_{D} of at least 1 nM, preferably 1 nM to 500 nM, at neutral pH and an off-rate constant k_{d} of at least 10⁻⁴ sec⁻¹, preferably 10⁻⁴ to 10⁻¹ sec⁻¹, at an acidic pH, preferably pH 5.

In some embodiments, the anti-TfR antibody or antigen-binding fragment thereof of claim 1 has an off-rate constant k_{d} of 2×10⁻² to 2×10⁻⁴ sec⁻¹, preferably 2.0×10⁻³ sec⁻¹ at a neutral pH.

In another embodiment, the optimized transport function of certain TfR binders described herein preferably have a kₐ rate of at least 1.05×10⁵ and a k_{d} rate of at least 2.0×10⁻³ s⁻¹ or faster at physiologic acidic pH (e.g., 7.4). The aforementioned pH, KD, kₐ and k_{d} parameters reflect optimized transcytosis conditions only and in no way limit our findings that TfR-mediated transport, of certain molecules conjugated to certain TfR binders herein, may nonetheless occur outside of the preferred parameters described.

In one general aspect, the application relates to an antibody or antigen-binding fragment thereof for delivering an agent to the brain of a subject in need thereof, wherein the antibody or antigen-binding fragment thereof binds to transferrin receptor (TfR), preferably a human transferrin receptor 1 (huTfR1), comprising
(1) a heavy chain variable region comprising heavy chain complementarity determining regions (HCDRs) HCDR1, HCDR2 and HCDR3, and a light chain variable region comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
   (i) SEQ ID NOs: 292, 293, 294, 295, 296, and 297, respectively;
   (ii) SEQ ID NOs: 279, 280, 281, 282, 283 and 284, respectively;
   (iii) SEQ ID NOs: 29, 30, 31, 32, 33 and 34, respectively;
   (iv) SEQ ID NOs: 57, 58, 59, 60, 61 and 62, respectively;
   (v) SEQ ID NOs: 85, 86, 87, 88, 89 and 90, respectively;
   (vi) SEQ ID NOs: 110, 111, 112, 113, 114 and 115, respectively;
   (vii) SEQ ID NOs: 135, 136, 137, 138, 139 and 140, respectively;
   (viii) SEQ ID NOs: 191, 192, 193, 194, 195 and 196, respectively;
   (ix) SEQ ID NOs: 244, 245, 246, 247, 248 and 249, respectively;
   (x) SEQ ID NOs: 263, 264, 265, 266, 267 and 268, respectively;
   (xi) SEQ ID NOs: 345, 346, 347, and 348, WAS, and SEQ ID NO: 350, respectively;
   (xii) SEQ ID NOs: 355, 356, 357, and 358, TLS, and SEQ ID NO: 360, respectively;
   (xiii) SEQ ID NOs: 365, 366, 367, and 368, TVS, and SEQ ID NO: 370, respectively;
   (xiv) SEQ ID NOs: 375, 376, 377, and 378, GAS, and SEQ ID NO: 380, respectively;
   (xv) SEQ ID NOs: 385, 386, 387, and 388, KVS, and SEQ ID NO: 390, respectively;
   (xvi) SEQ ID NOs: 395, 396, 377, and 398, SAS, and SEQ ID NO: 400, respectively;
   (xvii) SEQ ID NOs: 405, 406, 407, and 408, AAS, and SEQ ID NO: 410, respectively;
   (xviii) SEQ ID NOs: 415, 416, 417, and 418, QDN, and SEQ ID NO: 420, respectively;
   (xix) SEQ ID NOs: 425, 426, 427, and 428, AAS, and SEQ ID NO: 430, respectively;
   (xx) SEQ ID NOs: 435, 436, 437, and 438, ASS, and SEQ ID NO: 440, respectively;
   (xxi) SEQ ID NOs: 445, 446, 447, and 448, AAS, and SEQ ID NO: 450, respectively;
   (xxii) SEQ ID NOs: 455, 456, 457, and 458, DNN, and SEQ ID NO: 460, respectively;
   (xxiii) SEQ ID NOs: 465, 466, 467, and 468, GAS, and SEQ ID NO: 470, respectively;
   (xxiv) SEQ ID NOs: 475, 476, 477, and 478, QDR, and SEQ ID NO: 480, respectively;
   (xxv) SEQ ID NOs: 485, 486, 487, and 488, QDT, and SEQ ID NO: 490, respectively;
   (xxvi) SEQ ID NOs: 495, 496, 497, and 498, DDS, and SEQ ID NO: 500, respectively;
   (xxvii) SEQ ID NOs: 505, 506, 507, and 508, QDR, and SEQ ID NO: 510, respectively;
   (xxviii) SEQ ID NOs: 515, 516, 517, and 518, QDN, and SEQ ID NO: 520, respectively;
   (xxix) SEQ ID NOs: 525, 526, 527, and 528, EVS, and SEQ ID NO: 530, respectively;
   (xxx) SEQ ID NOs: 535, 536, 537, and 538, QDT, and SEQ ID NO: 540, respectively; or
   (xxxi) SEQ ID NOs: 545, 546, 547, and 548, QDR, and SEQ ID NO: 550, respectively; or
(2) a single variable domain on a heavy chain (VHH) comprising heavy chain complementarity determining regions (HCDRs) HCDR1, HCDR2 and HCDR3 having the amino acid sequences of:
   (i) SEQ ID NOs: 7, 8 and 9, respectively;
   (ii) SEQ ID NOs: 317, 318 and 319, respectively;
   (iii) SEQ ID NOs: 324, 325 and 326, respectively;
   (iv) SEQ ID NOs: 331, 332 and 333, respectively; or
   (v) SEQ ID NOs: 338, 339 and 340, respectively.
   In certain embodiments, the application relates to an anti-TfR VHH fragment comprising an amino acid sequence having at least 80%, such as at least 85%, 90%, 95% or 100%, sequence identity to SEQ ID NO: 6, 316, 323, 330, or 337.

In other embodiments, the application relates to an anti-TfR single-chain variable fragment (scFv) comprising a heavy chain variable region covalently linked to a light chain variable region via a linker, preferably, the linker has the amino acid sequence of SEQ ID NO: 314. More preferably, the scFv comprises an amino acid sequence having at least 80%, such as at least 85%, 90%, 95% or 100%, sequence identity to the amino acid sequences of SEQ ID NO: 278, 291, 28, 56, 84, 109, 134, 162, 190, 218, 243, 262, 344, 354, 364, 374, 384, 394, 404, 414, 424, 434, 444, 454, 464, 474, 484, 494, 504, 514, 524, 534 or 544.

Another aspect of the application relates to a conjugate comprising an anti-TfR antibody or antigen-binding fragment thereof of the application coupled to a therapeutic or diagnostic agent, such as a neurological disorder drug or an agent for detecting a neurological disorder. Preferably, the therapeutic or diagnostic agent is a second antibody or an antigen binding fragment thereof that binds to a brain target.

In certain embodiments, the application relates to a fusion construct comprising an anti-TfR antibody or antigen-binding fragment thereof of the application covalently linked to a second antibody or an antigen binding fragment thereof that binds to a brain target, such as a brain target selected from the group consisting of beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

In certain embodiments, a fusion construct of the application comprises a second antibody or antigen binding fragment thereof that binds to Tau and comprises HCDR1, HCDR2, HCDR3, LCDR1, LCDR2 and LCDR3 having the amino acid sequences of SEQ ID NOs: 554 to 559, respectively. Preferably, the second antibody is a monoclonal antibody comprising a heavy chain having the amino acid sequence of SEQ ID NO: 310 and a light chain having the amino acid sequence of SEQ ID NO: 311.

In one embodiment, a fusion construct of the application comprises an anti-TfR antibody or antigen-binding fragment thereof, preferably an anti-huTfR1 VHH or scFv fragment, of the application covalently linked, via a linker, to the carboxyl terminus of only one of the two heavy chains of a second antibody or antigen binding fragment thereof that binds to a brain target. Preferably, the linker has the amino acid sequence of SEQ ID NO: 312 or SEQ ID NO: 313.

In certain embodiments, each of the two heavy chains of the second antibody or antigen binding fragment thereof comprises a modified constant heavy chain 3 (CH3) domain as compared to a wild-type CH3 domain to facilitate the formation of a heterodimer between the two heavy chains. Any mutation that facilitates the formation of a heterodimer between the two heavy chains can be used. Preferably, the modified CH3 domain of the first heavy chain comprises amino acid modifications at positions T350, L351, F405, and Y407, and the modified CH3 domain of the second heavy chain comprises amino acid modifications at positions T350, T366, K392 and T394. Preferably, the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W. More preferably, the modified heterodimeric CH3 domain of the first heavy chain comprises mutations T350V, L351Y, F405A and Y407V, and the modified heterodimeric CH3 domain of the second heavy chain comprises mutations T350V, T366L, K392L and T394W. The numbering of amino acid residues in the antibody throughout the specification is performed according to the EU index as described in Kabat et al., Sequences of Proteins of Immunological Interest, 5th Ed. Public Health Service, National Institutes of Health, Bethesda, Md. (1991), unless otherwise explicitly stated.

In certain embodiments, the fragment crystallizable region (Fc region) of the second antibody or antigen binding fragment thereof contains substitutions that alter (increase or diminish), preferably eliminate, effector function, such as antibody dependent cellular cytotoxicity (ADCC) and/or complement dependent cytotoxicity (CDC). Preferably, the Fc region of the second antibody or antigen binding fragment thereof comprises one or more amino acid modifications that decrease or abolish the binding of the second antibody or antigen binding fragment thereof to Fc gamma receptors (FcγR) and avoid effector function mediated toxicity. For example, the Fc region of the second antibody or antigen binding fragment thereof can comprise one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat.

In certain embodiments, the Fc region of the second antibody or antigen binding fragment thereof contains substitutions that alter (increase or diminish), preferably increase, the binding of the second antibody or antigen binding fragment thereof to neonatal Fc receptor (FcRn). Preferably the one or more mutations enhance the binding at an acidic pH, more preferably the Fc has the M252Y/S254T/T256E (YTE) mutations, wherein the numbering of amino acid residues is according to the EU index as set forth in Kabat.

In certain embodiments, a fusion construct of the application comprises:
(1) a first heavy chain having an amino acid sequence that is at least 80%, such as at least 85%, 90%, 95% or 100%, identical to an amino acid sequence selected from the group consisting of SEQ ID NOs: 301, 304, 307, 285, 288, 298, 10, 13, 16, 19, 22, 25, 35, 38, 41, 44, 47, 50, 53, 63, 66, 69, 72, 75, 78, 81, 91, 94, 97, 100, 103, 106, 116, 119, 122, 125, 128, 131, 141, 144, 147, 150, 153, 156, 159, 169, 172, 175, 178, 181, 184, 187, 197, 200, 203, 206, 209, 212, 215, 225, 228, 231, 234, 237, 240, 250, 252, 256, 259, 269, 272, 275, 320, 327, 334, 341, 351, 361, 371, 381, 391, 401, 411, 421, 431, 441, 451, 461 and 471;
(2) two light chains each independently having an amino acid sequence that is at least 80%, such as at least 85%, 90%, 95% or 100%, identical to an amino acid sequence selected from the group consisting of 302, 305, 308, 286, 289, 299, 11, 14, 17, 20, 23, 26, 36, 39, 42, 45, 48, 51, 54, 64, 67, 70, 73, 76, 79, 82, 92, 95, 98, 101, 104, 107, 117, 120, 123, 126, 129, 132, 142, 145, 148, 151, 154, 157, 160, 170, 173, 176, 179, 182, 185, 188, 198, 201, 204, 207, 210, 213, 216, 226, 229, 232, 235, 238, 241, 251, 253, 257, 260, 270, 273 276, 321, 328, 335, 342, 352, 362, 372, 382, 392, 402, 412, 422, 432, 442, 452, 462 and 472, respectively; and
(3) a second heavy chain having an amino acid sequence that is at least 80%, such as at least 85%, 90%, 95% or 100%, identical to an amino acid sequence selected from the group consisting of 303, 306, 309, 287, 290, 300, 12, 15, 18, 21, 24, 27, 37, 40, 43, 46, 49, 52, 55, 65, 68, 71, 74, 77, 80, 83, 93, 96, 99, 102, 105, 108, 118, 121, 124, 127, 130, 133, 143, 146, 149, 152, 155, 158, 161, 171, 174, 177, 180, 183, 186, 189, 199, 202, 205, 208, 211, 214, 217, 227, 230, 233, 236, 239, 242, 252, 254, 258, 261, 271, 274, 277, 322, 329, 336, 343, 353, 363, 373, 383, 393, 403, 413, 423, 433, 443, 453, 463 and 473, respectively.

Another general aspect of the application relates to an isolated nucleic acid encoding the antibody or antigen-binding fragment, a conjugate, or a fusion construct of the application. Also provided is a vector comprising the isolated nucleic acid of the application, a host cell comprising the nucleic acid or the vector of the application.

Another general aspect of the application relates to a method of producing the antibody or antigen-binding fragment, a conjugate, or a fusion construct of the application. The method comprises culturing a cell comprising a nucleic acid of the application under conditions to produce the antibody or antigen-binding fragment, the conjugate or the fusion construct, and recovering the antibody or antigen-binding fragment, the conjugate or the fusion construct from the cell or cell culture.

Further provided is a pharmaceutical composition comprising a conjugate or a fusion construct of the application and a pharmaceutically acceptable carrier.

Another general aspect of the application relates to a method of treating or detecting a neurological disorder in a subject in need thereof, comprising administering to the subject an effective amount of an anti-TfR antibody or antigen binding fragment thereof, a conjugate or a fusion construct, or a pharmaceutical composition of the application. Preferably, the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

Preferably, the antibody or antigen binding fragment thereof, the conjugate, the fusion construct or the pharmaceutical composition of the application is administered intravenously.

Also described is a method of delivering a therapeutic or diagnostic agent to the brain of a subject in need thereof, comprising administering to the subject a conjugate comprising the therapeutic or diagnostic agent coupled to an anti-TfR antibody or antigen-binding fragment thereof of the application. Preferably, the therapeutic or diagnostic agent is a second antibody or an antigen binding fragment thereof that binds to a brain target. More preferably, the administration of the therapeutic or diagnostic agent coupled to an anti-TfR antibody or antigen-binding fragment thereof of the application to the brain of a subject results in reduced Fc-mediated effector function and/or does not induce rapid reticulocyte depletion, as compared to the administration of the therapeutic or diagnostic agent not coupled to the anti-TfR antibody or antigen-binding fragment thereof.

Yet another general aspect of the invention relates to a method of inducing antibody dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, an antigen-binding fragment thereof according to an embodiment of the invention, wherein the therapeutic antibody or antigen binding fragment thereof does not have effector function, for example, the therapeutic antibody or antigen binding fragment thereof comprises one or more amino acid modifications at positions L234, L235, D270, N297, E318, K320, K322, P331, and P329, such as one, two or three mutations of L234A, L235A and P331S, wherein the numbering of amino acid residues is according to the EU index as set forth in Kabat. Preferably, the therapeutic antibody or antigen binding fragment thereof binds specifically to tau aggregates.

Other aspects, features and advantages of the invention will be apparent from the following disclosure, including the detailed description of the invention and its preferred embodiments and the appended claims.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise embodiments shown in the drawings.

FIG. 1 is an illustration of tripod mAb format (also referred to as a TTP mAb) used for the brain delivery platform.

FIG. 2 is an image showing internalization of tripod mAbs in human brain endothelial cells. Tripod mAbs are stained red, nucleus is blue, and actin green.

FIG. 3 is a graph showing pH dependent binding, which was assessed by comparing off-rates at pH 7.4 to off-rate as pH was reduced to 6.5 and 6.0. Tripod mAbs were scored positive if the off-rate was faster as the pH decreased.

FIG. 4 is an image showing internalization of the tripod mAb BBBB383, in human brain endothelial cells. Tripod mAbs are stained red, nucleus is blue, and actin green.

FIG. 5A-FIG. 5B are graphs showing plasma (FIG. 5A) and brain (FIG. 5B) PK of BBBB383 and BBBB426. Brain shuttle containing anti-BACE mAbs, BBBB383 and BBBB426 were compared with BBBB456 (anti-BACE mAb without the brain shuttle). Symbols represent the average of 4 mice (at 4 and 24 hours) or 5 mice (at 72 hours).

FIG. 6 is a graph showing Aβ1-40 concentrations in brain following treatment with BBBB383 and BBBB426. Brain shuttle containing anti-BACE mAbs, BBBB383 and BBBB426, were compared with BBBB456 (anti-BACE mAb without the brain shuttle). Symbols represent the average of 4 mice (at 4 and 24 hours) or 5 mice (at 72 hours).

FIG. 7A-FIG. 7B are graphs showing plasma (FIG. 7A) and brain (FIG. 7B) PK of brain shuttle anti-BACE mAbs. Brain shuttle containing anti-BACE mAbs were compared with BBBB456 (anti-BACE mAb without the brain shuttle, solid diamond with dotted line). Each symbol represents the average of two mice per timepoint.

FIG. 8 is a graph showing Aβ1-40 concentrations in brain following treatment with brain shuttle mAbs. Brain shuttle containing anti-BACE mAbs, were compared with BBBB456 (anti-BACE mAb without the brain shuttle, solid diamond with dotted line). Dose dependent decrease in AB levels was observed for all brain shuttles except BBBB983. Each symbol represents the average of two mice per timepoint.

FIG. 9 is an image showing internalization of tripod mAb BBB-00489 in human brain endothelial cells. Tripod mAbs are stained red and actin green.

FIG. 10 are graphs showing brain pharmacokinetics in cynomolgus monkey. Cynomolgus monkeys were dosed 10 mg/kg intravenously with three TTP mAbs, BBBB1134 and BBBB1136 (left) and BBBB1133 (right), and compared to control mAb, PT1B844. Brain exposure measured 72 hours following dosing (n=3 cynomolgus monkeys/mAb). Brain concentration was determined for the mAbs across a variety of areas and averaged across animals. Each symbol represents a region of the brain.

FIG. 11 is a graph showing brain concentration of a brain shuttle-containing mAb as compared to the non-brain shuttle control in different regions. Individual points represent each animal (n=3).

FIG. 12 is a graph showing plasma concentration of mAbs dosed i.v. at 4, 24 and 72 hours. All brain shuttle mAbs had faster clearance than non-brain shuttle mAbs. Individual points represent each animal (n=3).

FIG. 13 is a graph showing reticulocyte depletion observed during the cyno study for BBBB1134 but not the other mAbs, confirming the impact of Fc function on TfR binding mAbs and reticulocyte depletion.

FIG. 14A-FIG. 14C: Brain pharmacokinetics and pharmacodynamics of tripod mAbs in huTfR knock-in mice, human TfR knock-in mice were dosed 10 mg/kg intravenously with a panel of tripod mAbs (BBBBx) compared with one control mAb, and brain exposure was assessed at 24 hours:
FIG. 14A: a range of enhanced exposure was observed from no enhancement (BBBB974, open square) to 10.5× (BBBB978, open triangle) (n=2 mice, symbols represent each individual animal with the bar representing the mean and error bars standard deviation).
FIG. 14B: the tripod mAb off-rates correlated well with brain exposure, with an off-rate that was neither too fast nor too slow observed to be optimal.
FIG. 14C: brain pharmacodynamics of the mAb, anti-BACE antagonist mAb, were assessed and a strong PK/PD relationship was observed in the brain for all tripod mAbs, except BBBB983. BBBB983 had enhanced brain exposure (5.5×) but similar concentration of Aβ₁₋₄₀ as the control mAb (each triangle represents an individual). It is hypothesized that the slow-neutral off-rate is preventing diffusion in the brain to the target.

FIG. 15 is a graph showing mAb mediated uptake into microglial phagosomes. All brain shuttle mAbs promoted more efficient uptake into phagosomes than the non-brain shuttle mAb, PT1B844. Within the brain shuttle mAbs those with full effector function (BBBB1131, 1134 and 1046) were more efficient than those without effector function.

FIG. 16 is a graph showing mAb mediated uptake into macrophage phagosomes. All brain shuttle mAbs promoted more efficient uptake into phagosomes than the non-brain shuttle mAb, B21M-IgG1.

FIG. 17A-FIG. 17F: Brain pharmacokinetics in cynomolgus monkey demonstrate enhanced brain delivery of therapeutic mAb.
FIG. 17A: cynomolgus monkeys were dosed 10 mg/kg intravenously with two tripod mAbs, BBBB1134 and BBBB1136, and one control mAb, PT1B844. Brain exposure measured 72 hours following dosing (n=3 cynomolgus monkeys/mAb. Symbols represent each individual animal with the bar representing the mean and error bars standard deviation). Enhanced brain exposure was observed for both brain shuttle mAbs across all regions of the brain assessed.
FIG. 17B: a 7× and 11× enhancement in brain concentration was observed for BBBB1134 and BBBB136, respectively, compared with the control mAb.
FIG. 17C: the plasma exposure over 72 hours demonstrated target-mediated drug disposition for the tripod mAbs with accelerated clearance observed compared with the control mAb. The tripod mAbs differ in their binding affinity for FcRn, with BBBB1136 containing the high binding affinity “YTE” mutations; BBBB1136 (triangle) had approximately 2× enhanced plasma concentration at 72 hours compared with BBBB1134 (open square).
FIG. 17D: in vitro ADCC activity of the tripod mAbs (BBBB1134 and BBBB1136) compared with the positive control, BBBB175 high affinity anti-TfR binding IgG1 mAb, and negative control, CNT03930, an IgG1 mAb that does not bind the target cells. BBBB1134, IgG1 mAb, potentiates robust ADCC of target cells with both human and cyno PBMCs. BBBB1136, an effector function silent IgG1 mAb, was observed to have no ADCC activity.
FIG. 17E: SPR binding data of BBBB1134 and BBBB1136 for the complement component 1q (C1q). BBBB1134 binds C1q while BBBB1136 does not.
FIG. 17F: Reticulocyte depletion observed in the cynomolgus monkey PK study. Reticulocyte loss 2 days following dosing was not observed for the control mAb or BBBB1136, while robust depletion was observed following treatment with BBBB1134 (symbols represent the individual animals, bars the average and error bars the standard deviation).

FIG. 18A-FIG. 18D: Brain and Serum pharmacokinetics of repeat dosing and dose response of BBBB1133 in cynomolgus monkey:
FIG. 18A: Cynomolgus monkeys were dosed intravenously with either 2 mg/kg, 10 mg/kg or 30 mg/kg with BBBB1133 and brain exposure assessed at 1, 7 or 15 days following (n=3 monkeys/mAb and timepoint. Symbols represent the average brain concentration and error bars the standard deviation). Linear brain PK was observed between 2 and 10 mg/kg but nonlinear PK observed between 10 and 30 mg/kg, suggesting that 30 mg/kg is a saturating dose for the TfR.
FIG. 18B: Serum concentration of BBBB1133 was measured throughout the study (1, 6 hours post-dosing and on days 1, 2, 4, 10 and 14). Linear pharmacokinetics was observed at all three doses. A T_{1/2}=6 days was determined for BBBB1133 in serum.
FIG. 18C: Cynomolgus monkeys were dosed intravenously weekly for three weeks with BBBB1133 at either 2 mg/kg, 10 mg/kg or 30 mg/kg. Brain exposure assessed at 1, 7, 15, or 21 days following dosing (n=3 monkeys/mAb and timepoint. Symbols represent the average brain concentration and error bars the standard deviation). Linear brain PK was observed between 2 and 10 mg/kg but nonlinear PK observed between 10 and 30 mg/kg, suggesting that 30 mg/kg is a saturating dose for the TfR. Evidence for accumulation was observed at the 30 mg/kg dose.
FIG. 18D: Serum concentration of BBBB1133 was measured throughout the study (1, 6 hours post first dose and on days 1, 2, 4, 10, 14, 14.02, 14.25, 15, 16, 18 and 21). Linear pharmacokinetics was observed at all three doses with no evidence for PK tolerance with repeat dosing.

FIG. 19A-FIG. 19C: Non-classical, non-FcγR mediated ADP promotes the efficient phagocytosis of Tau aggregates in human microglia:
FIG. 19A: To assess the potential of the effector function impaired IgG1 tripod mAbs, BBBB1133 and BBBB1136, to promote uptake of Tau aggregates in microglia, human iPSC derived microglia were incubated with mAbs and biotinylated phospho-tau oligomers labeled with streptavidin Alexa Flour 488 (AF488). At 4 hours post incubation, cells were washed, fixed, permeabilized, stained and imaged using confocal microscopy. Cells containing tau aggregates that co-localized with Lamp-1 stained lysosomes were quantitated. BBBB1133 and BBBB1136 promoted more efficient uptake and lysosomal trafficking than the anti-Tau WT IgG1 mAb, PT1B844.
FIG. 19B: Uptake of Tau oligomers can be blocked with excess of soluble TfR ECD but is not impacted by addition of soluble Fc, demonstrating the uptake occurs through TfR.
FIG. 19C: Human iPSc-derived microglia were incubated with Alexa Fluor 488-labeled phosphoTau peptide (green) in the presence of PT1B844 or BBBB1133 for 4 hours. After fixation, cells were stained with antibodies against Clathrin, EEA1, Rab17 or Lamp1, and detected with Alexa Fluor 647-secondary antibodies (red). Cells were imaged using a Perkin Elmer Opera Phenix, 60× magnification, confocal mode. Representative cell images at the 2 μm plane are shown. Scale bar=10 μm. Arrows point at the colocalization area detailed in insets. Third column for each phosphoTau-antibody treatment is the merged result of the other two columns. Cells were also stained and imaged with DAPI to detect nuclei and hcs Cellmask orange to detect cytoplasm (not shown).

FIG. 20A-FIG. 20E: Non-classical, non-FcγR mediated ADP promotes the efficient phagocytosis of Tau aggregate derived from human AD patient brains in human macrophages and microglia:
FIG. 20A: Human monocyte-derived macrophages were incubated with Tau aggregates and BBBB1133 (open square) and the control anti-Tau mAb, PT1B844 (circle). The amount of pTau remaining in the culture supernatant was quantified with time. Similar degradation of pTau was observed up 8 hours, at which point the PT1B844-mediated ADP stalls while the BBBB1133-mediated ADP continues to promote degradation.
FIG. 20B: A similar trend was observed using human iPSC-derived microglia, with BBBB1133 (open square) potentiating more robust degradation of pTau with time compared with PT1B844. The mechanism of BBBB1133-mediated pTau degradation was demonstrated to occur through the TfR by blocking degradation using excess amount of soluble TfR ECD.
FIG. 20C-FIG. 20E: Supernatants from the microglia experiment were assess for cytokine concentrations. PT1B844-mediated pTau ADP simulated the release of proinflammatory cytokines, TFNα (FIG. 20C), IL6 (FIG. 20D) and IL1β (FIG. 20E), while BBBB1133 did not simulate similar release.

FIG. 21: Co-injection of PHFs with the indicated tau antibodies reduced the induction of tau pathology:
FIG. 21A: Partial dependency of the model on Fc-dependent activity is demonstrated by the statistically significant differences in neutralization of Tau by the mouse IgG2a.
FIG. 21B: Both anti-Tau mAbs neutralized Tau seeding compared with the isotype control. No statistical difference was observed between the mAb and the TTP mAb, with the TTP mAb having slightly improved neutralization compared with the mAb, demonstrating that non-classical ADP mechanism is functional in vivo.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

The term “antibody” herein is used in the broadest sense and specifically includes full-length monoclonal antibodies, polyclonal antibodies, and, unless otherwise stated or contradicted by context, antigen-binding fragments, antibody variants, and multispecific molecules thereof, so long as they exhibit the desired biological activity. Generally, a full-length antibody is a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. General principles of antibody molecule structure and various techniques relevant to the production of antibodies are provided in, e.g., Harlow and Lane, ANTIBODIES: A LABORATORY MANUAL, Cold Spring Harbor Laboratory Press, Cold Spring Harbor, N. Y., (1988).

Depending on the amino acid sequence of the constant domain of their heavy chains, full length antibodies can be assigned to different “classes”. There are five major classes of full-length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

An “antibody” can also be a single variable domain on a heavy chain (VHH) antibody, also referred to as a heavy chain only antibody (HcAb), which are devoid of light chains and can be naturally produced by camelids or sharks. The antigen binding portion of the HcAb is comprised of a VHH fragment.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including YO and NSO cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia, coli cells or Bacillus subtilis cells, etc.

An “antigen-binding fragment” of an antibody is a molecule that comprises a portion of a full-length antibody which is capable of detectably binding to the antigen, typically comprising one or more portions of at least the VH region. Antigen-binding fragments include multivalent molecules comprising one, two, three, or more antigen-binding portions of an antibody, and single-chain constructs wherein the VL and VH regions, or selected portions thereof, are joined by synthetic linkers or by recombinant methods to form a functional, antigen-binding molecule. Antigen-binding fragments can also be a single-domain antibody (sdAb), also known as a nanobody, which is an antibody fragment consisting of a single monomeric variable antibody domain (VHH). While some antigen-binding fragments of an antibody can be obtained by actual fragmentation of a larger antibody molecule (e.g., enzymatic cleavage), most are typically produced by recombinant techniques. The antibodies of the invention can be prepared as full-length antibodies or antigen-binding fragments thereof. Examples of antigen-binding fragments include Fab, Fab′, F(ab)₂, F(ab′)₂, F(ab)₃, Fv (typically the VL and VH domains of a single arm of an antibody), single-chain Fv (scFv, see e.g., Bird et al., Science 1988; 242:423-426; and Huston et al. PNAS 1988; 85:5879-5883), dsFv, Fd (typically the VH and CH1 domain), and dAb (typically a VH domain) fragments; VH, VL, VHH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23:1126-1136; WO2005040219, and published U.S. Patent Applications 20050238646 and 20020161201. Antibody fragments can be obtained using conventional recombinant or protein engineering techniques, and the fragments can be screened for antigen-binding or other function in the same manner as are intact antibodies.

Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al., Science, 229:81 (1985)). However, these fragments can now be produced directly by recombinant host cells. Alternatively, Fab′-SH fragments can be directly recovered from E. coli and chemically coupled to form F(ab′)2 fragments (Carter et al., Bio/Technology, 10:163-167 (1992)). According to another approach, F(ab′)2 fragments can be isolated directly from recombinant host cell culture. In other embodiments, the antibody of choice is a single-chain Fv fragment (scFv). See WO 1993/16185; U.S. Pat. Nos. 5,571,894; and 5,587,458. The antibody fragment may also be a “linear antibody”, e.g., as described in U.S. Pat. No. 5,641,870, for example. Such linear antibody fragments can be monospecific or bispecific.

The term “antibody derivative” as used herein refers to a molecule comprising a full-length antibody or an antigen-binding fragment thereof, wherein one or more amino acids are chemically modified or substituted. Chemical modifications that can be used in antibody derivative includes, e.g., alkylation, PEGylation, acylation, ester formation or amide formation or the like, e.g., for linking the antibody to a second molecule. Exemplary modifications include PEGylation (e.g., cysteine-PEGylation), biotinylation, radiolabeling, and conjugation with a second agent (such as a cytotoxic agent).

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al); and/or increased or diminished serum half-life (see, for example, WO00/42072, Presta, L.).

A “multispecific molecule” comprises an antibody, or an antigen-binding fragment thereof, which is associated with or linked to at least one other functional molecule (e.g. another peptide or protein such as another antibody or ligand for a receptor) thereby forming a molecule that binds to at least two different binding sites or target molecules. Exemplary multispecific molecules include bi-specific antibodies and antibodies linked to soluble receptor fragments or ligands.

The term “human antibody”, as used herein, is intended to include antibodies having variable regions in which both the framework and CDR regions are derived from (i.e., are identical or essentially identical to) human germline immunoglobulin sequences. Furthermore, if the antibody contains a constant region, the constant region also is “derived from” human germline immunoglobulin sequences. The human antibodies of the invention can include amino acid residues not encoded by human germline immunoglobulin sequences (e.g., mutations introduced by random or site-specific mutagenesis in vitro or by somatic mutation in viva). However, the term “human antibody”, as used herein, is not intended to include antibodies in which CDR sequences derived from the germline of another mammalian species, such as a mouse, have been grafted onto human framework sequences.

A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992), WO 92/02190, US Patent Application 20060073137, and U.S. Pat. Nos. 6,750,325, 6,632,927, 6,639,055, 6,548,640, 6,407,213, 6,180,370, 6,054,297, 5,929,212, 5,895,205, 5,886,152, 5,877,293, 5,869,619, 5,821,337, 5,821,123, 5,770,196, 5,777,085, 5,766,886, 5,714,350, 5,693,762, 5,693,761, 5,530,101, 5,585,089, and 5,225,539.

The term “hypervariable region” when used herein refers to the amino acid residues of an antibody that are responsible for antigen binding. The hypervariable region generally comprises amino acid residues from a “complementarity-determining region” or “CDR” (residues 24-34 (L1), 50-56 (L2) and 89-97 (L3) in the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide can contain fewer or additional amino acids corresponding to a shortening of, or insertion into, a FR or CDR of the variable domain. For example, a heavy chain variable domain can include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence.

“Framework region” or “FR” residues are those VH or VL residues other than the CDRs as herein defined.

An “epitope” or “binding site” is an area or region on an antigen to which an antigen-binding peptide (such as an antibody) specifically binds. A protein epitope can comprise amino acid residues directly involved in the binding (also called the immunodominant component of the epitope) and other amino acid residues, which are not directly involved in the binding, such as amino acid residues which are effectively blocked by the specifically antigen binding peptide (in other words, the amino acid residue is within the “solvent-excluded surface” and/or “footprint” of the specifically antigen binding peptide).

A “paratope” is an area or region of an antigen-binding portion of an antibody that specifically binds an antigen. Unless otherwise stated or clearly contradicted by context, a paratope can comprise amino acid residues directly involved in epitope binding, several of which are typically in CDRs, and other amino acid residues, which are not directly involved in the binding, such as amino acid residues which are effectively blocked by the specifically bound antigen (in other words, the amino acid residue is within the “solvent-excluded surface” and/or “footprint” of the specifically bound antigen).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al, J. Chromatogr. B 848:79-87 (2007).

The term “administering” with respect to the methods of the invention, means a method for therapeutically or prophylactically preventing, treating or ameliorating a syndrome, disorder or disease as described herein by using a conjugate of the invention or a form, composition or medicament thereof. Such methods include administering an effective amount of said antibody, antigen-binding fragment thereof, or conjugate, or a form, composition or medicament thereof at different times during the course of a therapy or concurrently in a combination form. The methods of the invention are to be understood as embracing all known therapeutic treatment regimens.

The ability of a target antibody to “block” the binding of a target molecule to a natural target ligand, means that the antibody, in an assay using soluble or cell-surface associated target and ligand molecules, can detectably reduce the binding of a target molecule to the ligand in a dose-dependent fashion, where the target molecule detectably binds to the ligand in the absence of the antibody.

The “blood-brain barrier” or “BBB” refers a physiological barrier between the peripheral circulation and the brain and spinal cord which is formed by tight junctions within the brain capillary endothelial plasma membranes, creating a tight barrier that restricts the transport of molecules into the brain. The BBB can restrict the transport of even very small molecules such as urea (60 Daltons) into the brain. Examples of the BBB include the BBB within the brain, the blood-spinal cord barrier within the spinal cord, and the blood-retinal barrier within the retina, all of which are contiguous capillary barriers within the CNS. The BBB also encompasses the blood-CSF barrier (choroid plexus) where the barrier is comprised of ependymal cells rather than capillary endothelial cells.

A “blood-brain barrier receptor” (abbreviated “R/BBB” herein) is an extracellular membrane-linked receptor protein expressed on brain endothelial cells which is capable of transporting molecules across the BBB or be used to transport exogenous administrated molecules. Examples of R/BBB include, but are not limited to, transferrin receptor (TfR), insulin receptor, insulin-like growth factor receptor (IGF-R), low density lipoprotein receptors including without limitation low density lipoprotein receptor-related protein 1 (LRP1) and low density lipoprotein receptor-related protein 8 (LRP8), and heparin-binding epidermal growth factor-like growth factor (HB-EGF). An exemplary R/BBB herein is transferrin receptor (TfR).

The “central nervous system” or “CNS” refers to the complex of nerve tissues that control bodily function, and includes the brain and spinal cord.

A “conjugate” as used herein refer to a protein covalently linked to one or more heterologous molecule(s), including but not limited to a therapeutic peptide or protein, an antibody, a label, or a neurological disorder drug.

As used herein the term “coupled” refers to the joining or connection of two or more objects together. When referring to chemical or biological compounds, coupled can refer to a covalent connection between the two or more chemical or biological compounds. By way of a non-limiting example, an antibody of the invention can be coupled with a peptide of interest to form an antibody coupled peptide. An antibody coupled peptide can be formed through specific chemical reactions designed to conjugate the antibody to the peptide. In certain embodiments, an antibody of the invention can be covalently coupled with a peptide of the invention through a linker. The linker can, for example, be first covalently connected to the antibody or the peptide, then covalently connected to the peptide or the antibody.

An “effective amount” or “therapeutically effective amount” of an agent, e.g., a pharmaceutical formulation, refers to an amount effective, at dosages and for periods of time necessary, to achieve the desired therapeutic or prophylactic result.

A “linker” as used herein refers to a chemical linker or a single chain peptide linker that covalently connects two different entities. A linker can be used to connect any two of an antibody or a fragment thereof, a blood brain barrier shuttle, a fusion protein and a conjugate of the present invention. The linker can connect, for example, the VH and VL in scFv, or the monoclonal antibody or antigen-binding fragment thereof with a therapeutic molecule, such as a second antibody. In some embodiment, if the monovalent binding entity comprises a scFv directed to TfR, preferably huTfR1, and the therapeutic molecule comprises an antibody directed to a CNS target, such as Tau, then the linker can connect the scFv to the antibody directed to Tau. Single chain peptide linkers, comprised of from 1 to 25 amino acids, such as 1, 2, 3, 4, 5, 6, 7, 8, 9, 10, 11, 12, 13, 14, 15, 16, 17, 18, 19, 20, 21, 22, 23, 24, or 25 amino acids, joined by peptide bonds, can be used. In certain embodiments, the amino acids are selected from the twenty naturally occurring amino acids. In certain other embodiments, one or more of the amino acids are selected from glycine, alanine, proline, asparagine, glutamine and lysine. Chemical linkers, such as a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain can also be used.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (including, but not limited to, Alzheimer disease and supranuclear palsy), prion diseases (including, but not limited to, bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS and/or brain, including brain metastases resulting from cancer elsewhere in the body).

A “neurological disorder drug” is a drug or therapeutic agent useful in treating or ameliorating the effects of one or more neurological disorder(s). Neurological disorder drugs of the invention include, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, or active fragments of any of the foregoing. Exemplary neurological disorder drugs of the invention are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, Glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

The term “pharmaceutical formulation” refers to a preparation which is in such form as to permit the biological activity of an active ingredient contained therein to be effective, and which contains no additional components which are unacceptably toxic to a subject to which the formulation would be administered.

As used herein, “pharmaceutically acceptable carrier or diluent” means any substance suitable for use in administering to an individual. For example, a pharmaceutically acceptable carrier can be a sterile aqueous solution, such as phosphate buffer saline (PBS) or water-for-injection.

As used herein, “pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

Pharmaceutically acceptable acidic/anionic salts for use in the invention include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid. Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, chloroprocaine, choline, cyclohexylamine, diethanolamine, ethylenediamine, lithium, L-lysine, magnesium, meglumine, N-methyl-D-glucamine, piperidine, potassium, procaine, quinine, sodium, triethanolamine, or zinc.

“Polypeptide” or “protein” means a molecule that comprises at least two amino acid residues linked by a peptide bond to form a polypeptide. Small polypeptides of less than 50 amino acids may be referred to as “peptides”.

The phrases “sequence identity” or “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

The term “substantially identical” in the context of two amino acid sequences means that the sequences, when optimally aligned, such as by the programs GAP or BESTFIT using default gap weights, share at least about 50 percent sequence identity. Typically sequences that are substantially identical will exhibit at least about 60, at least about 70, at least about 80, at least about 90, at least about 95, at least about 98, or at least about 99 percent sequence identity.

“Specific binding” or “specifically binds” or “binds” refer to antibody binding to an antigen or an epitope within the antigen with greater affinity than for other antigens. Typically, the antibody binds to the antigen or the epitope within the antigen with a dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with a K_{D} that is at least one hundred fold less than its K_{D} for binding to a non-specific antigen (e.g., BSA, casein). K_{D} is the equilibrium dissociation constant, a ratio of k_{off}/kₒₙ, between the antibody and its antigen. K_{D} and affinity are inversely related. The “on-rate” (kₒₙ) is a constant used to characterize how quickly the antibody binds to its target. The “off-rate” (k_{off}) is a constant used to characterize how quickly an antibody dissociates from its target. The dissociation constant K_{D} can be measured using standard procedures. For example, the K_{D} of an antibody can be determined by using surface plasmon resonance, such as by using a biosensor system, e.g., a Biacore® system, or by using bio-layer interferometry technology, such as an Octet RED96 system. The smaller the value of the K_{D} of an antibody, the higher affinity that the antibody binds to a target antigen. Antibodies that specifically bind to the antigen or the epitope within the antigen can, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

The term “subject” as used herein refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. When the subject is human, they can also be referred to as a “patient”.

The term “transferrin receptor” or “TfR,” as used herein, refers to a cell surface receptor necessary for cellular iron uptake by the process of receptor-mediated endocytosis. carrier protein for transferrin. A TfR is involved in iron uptake in vertebrates and is regulated in response to intracellular iron concentration. It imports iron by internalizing the transferrin-iron complex through receptor-mediated endocytosis. Two transferrin receptors in humans, transferrin receptor 1 and transferrin receptor 2, have been characterized. Both these receptors are transmembrane glycoproteins. TfR1 is a high affinity ubiquitously expressed receptor. TfR2 binds to transferrin with a 25-30-fold lower affinity than TfR1. The expression of TfR2 is restricted to certain cell types and is unaffected by intracellular iron concentrations. In one embodiment, the TfR is a human TfR comprising the amino acid sequence as in Schneider et al. Nature 311: 675-678 (1984), for example. It can have a molecular weight of about 180,000 Dalton, having two subunits each of apparent molecular weight of about 90,000 Dalton. Preferably, the TfR is a human TfR1.

A “target antigen” or “brain target,” as used herein, refers to an antigen and/or molecule expressed in the CNS, including the brain, which can be targeted with an antibody or small molecule. Examples of such antigens and/or molecules include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In some embodiments, the target antigen is BACE1. In some embodiments, the target antigen is Tau.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

Antibodies or immunoglobulins can be assigned to five major classes, namely IgA, IgD, IgE, IgG and IgM, depending on the heavy chain constant domain amino acid sequence. IgG is the most stable of the five types of immunoglobulins, having a serum half-life in humans of about 23 days. IgA and IgG are further sub-classified as the isotypes IgA1, IgA2, IgG1, IgG2, IgG3 and IgG4. Each of the four IgG subclasses has different biological functions known as effector functions. These effector functions are generally mediated through interaction with the Fc receptor (FcγR) and/or by binding C1q and fixing complement. Binding to FcγR can lead to antibody dependent cell mediated cytolysis or antibody-dependent cellular cytotoxicity (ADCC), whereas binding to complement factors can lead to complement mediated cell lysis or complement-dependent cytotoxicity (CDC). An anti-TfR antibody of the invention, or a therapeutic or diagnostic antibody to be conjugated or fused to the anti-TfR antibody can have no or minimal effector function, but retains its ability to bind FcRn, the binding of which can be a primary means by which antibodies have an extended in vivo half-life.

Binding of FcγR or complement (e.g., C1q) to an antibody is caused by defined protein-protein interactions at the so-called Fc part binding site. Such Fc part binding sites are known in the art. Such Fc part binding sites include, e.g., characterized by the amino acids L234, L235, D270, N297, E318, K320, K322, P331, and P329 (numbering according to EU index of Kabat). In some embodiment, an anti-TfR antibody of the invention, or a therapeutic or diagnostic antibody to be conjugated or fused to the anti-TfR antibody contains one or more substitutions in one or more Fc part binding sites to eliminate the effector function. For example, an anti-TfR antibody of the invention, or a therapeutic or diagnostic antibody to be conjugated or fused to the anti-TfR antibody can contain a Fc region containing one or more of the following substitutions: substitution of proline for glutamate at residue 233, alanine or valine for phenylalanine at residue 234 and alanine or glutamate for leucine at residue 235 (EU numbering, Kabat, E. A. et al. (1991) Sequences of Proteins of Immunological Interest, 5th Ed. U.S. Dept. of Health and Human Services, Bethesda, Md., NIH Publication no. 91-3242). Preferably, the antibody of interest contains one, two or three mutations of L234A, L235A and P331S (EU numbering, Kabat).

Antibodies of subclass IgG1, IgG2, and IgG3 usually show complement activation including C1q and C3 binding, whereas IgG4 does not activate the complement system and does not bind C1q and/or C3. Human IgG4 Fc region has reduced ability to bind FcγR and complement factors compared to other IgG sub-types. Preferably, an anti-TfR antibody of the invention, or a therapeutic or diagnostic antibody to be conjugated or fused to the anti-TfR antibody comprises a Fc region derived from human IgG4 Fc region. More preferably, the Fc region contains human IgG4 Fc region having substitutions that eliminate effector function. For example, removing the N-linked glycosylation site in the IgG4 Fc region by substituting Ala for Asn at residue 297 (EU numbering) is another way to ensure that residual effector activity is eliminated.

### Anti-TfR Antibodies and Antigen Binding Fragments Thereof

In one general aspect, the application relates to an antibody or an antigen binding fragment thereof that binds to a primate TfR, such as a human TfR or a monkey TfR, and the antibody or an antigen binding fragment thereof is optimized for delivering an agent to the brain of a subject in need thereof. The relationship between the binding affinity of an anti-TfR antibody to the TfR and transcytosis efficiency has been described previously as improved transcytosis with decreased affinity for TfR (Yu, Zhang et al. 2011, Sci Transl Med 3(84): 84ra44) The inventors of the present invention surprisingly discovered a more nuanced relationship between affinity and transcytosis efficiency than what has been previously described, with influence from both on- and off-rates impacting brain concentration. In particular, a neutral off-rate that is neither too fast nor too slow is required for optimal brain PK and PD of an agent (such as an mAb) to be efficiently delivered by the anti-TfR antibody or antigen binding fragment thereof.

Preferably, an anti-TfR antibody or antigen binding fragment thereof of the application is pH-sensitive, e.g., it has different binding affinities to TfR at different pHs. For example, an anti-TfR antibody of the application can bind to cell surface TfR at a neutral pH, such as physiological pH (e.g., pH 7.4), with high affinity, but upon internalization into an endosomal compartment, dissociates from TfR at an acidic pH, such as the relatively lower pH (pH 5.0-6.0). Affinity is a measure of the strength of binding between two moieties, e.g., an antibody and an antigen. Affinity can be expressed in several ways. One way is in terms of the dissociation constant (K_{D}) of the interaction. K_{D} can be measured by routine methods, include equilibrium dialysis or by directly measuring the rates of antigen-antibody dissociation and association, the k_{off} (k_{d} or k_{dis}) and kₒₙ(or ka) rates, respectively (see e.g., Nature, 1993 361:186-87). The ratio of k_{off}/kₒₙ cancels all parameters not related to affinity, and is equal to the dissociation constant K_{D} (see, generally, Davies et al., Annual Rev Biochem, 1990 59:439-473). Thus, a smaller K_{D} means a higher affinity. Another expression of affinity is Kₐ, which is the inverse of K_{D}, or kₒₙ/k_{off}. Thus, a higher Kₐ means a higher affinity. For example, an antibody or antigen binding fragment thereof for use in a composition and/or method of the application can be an antibody or fragment thereof that binds to a TfR with a K_{D} of 1 nanomolar (nM, 10⁻⁹ M) or more at a neutral pH (e.g., pH 6.8-7.8), such as a physiological pH (e.g., pH 7.4), and dissociates from TfR with a k_{dis} of 10⁻⁴ sec⁻¹ or more at an acidic pH (e.g., pH 4.5-6.0), such as pH5.0).

Accordingly, a general aspect of the application relates to an anti-TfR antibody or antigen-binding fragment thereof for delivering an agent to the brain of a subject in need thereof, wherein the anti-TfR antibody or antigen-binding fragment thereof binds to a transferrin receptor (TfR), preferably human TfR1, with a dissociation constant K_{D} of at least 1 nM, preferably 1 nM to 500 nM, at neutral pH and an off-rate constant k_{d} of at least 10⁻⁴ sec⁻¹, preferably 10⁻⁴ to 10⁻¹ sec⁻¹, at an acidic pH, preferably pH 5.

In one embodiment, the anti-TfR antibody or antigen-binding fragment thereof of the application has an off-rate constant k_{d} of 2×10⁻² to 2×10⁻⁴ sec⁻¹, such as 2×10⁻², 1×10⁻², 9×10⁻³, 8×10⁻³, 7×10⁻³, 6×10⁻³, 5×10⁻³, 4×10⁻³, 3×10⁻³, 2×10⁻³, 1×10⁻³, 9×10⁻⁴, 8×10⁻⁴, 7×10⁻⁴, 6×10⁻⁴, 5×10⁻⁴, 4×10⁻⁴, 3×10⁻⁴, 2×10⁻⁴ sec⁻¹, or any value in between, at the neutral pH.

In certain embodiments, the antibody or antigen binding fragment thereof that binds to human TfR is a single variable domain on a heavy chain (VHH) antibody comprising heavy chain complementarity determining regions (HCDRs) HCDR1, HCDR2 and HCDR3 having the amino acid sequences of:
(i) SEQ ID NOs: 7, 8 and 9, respectively;
(ii) SEQ ID NOs: 317, 318 and 319, respectively;
(iii) SEQ ID NOs: 324, 325 and 326, respectively;
(iv) SEQ ID NOs: 331, 332 and 333, respectively; or
(v) SEQ ID NOs: 338, 339 and 340, respectively.

Preferably, it is a VHH fragment comprising an amino acid sequence having at least 80%, such as at least 85%, 90%, 91%, 92%, 93%, 94%, 95%, 96%, 97%, 98%, 99% or 100%, sequence identity to SEQ ID NO: 6, 316, 323, 330, or 337.

In other embodiments, the antibody or antigen binding fragment thereof that binds to human TfR comprises a heavy chain variable region comprising heavy chain complementarity determining regions (HCDRs) HCDR1, HCDR2 and HCDR3, and a light chain variable region comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
(i) SEQ ID NOs: 292, 293, 294, 295, 296, and 297, respectively;
(ii) SEQ ID NOs: 279, 280, 281, 282, 283 and 284, respectively;
(iii) SEQ ID NOs: 29, 30, 31, 32, 33 and 34, respectively;
(iv) SEQ ID NOs: 57, 58, 59, 60, 61 and 62, respectively;
(v) SEQ ID NOs: 85, 86, 87, 88, 89 and 90, respectively;
(vi) SEQ ID NOs: 110, 111, 112, 113, 114 and 115, respectively;
(vii) SEQ ID NOs: 135, 136, 137, 138, 139 and 140, respectively;
(viii) SEQ ID NOs: 191, 192, 193, 194, 195 and 196, respectively;
(ix) SEQ ID NOs: 244, 245, 246, 247, 248 and 249, respectively;
(x) SEQ ID NOs: 263, 264, 265, 266, 267 and 268, respectively;
(xi) SEQ ID NOs: 345, 346, 347, and 348, WAS, and SEQ ID NO: 350, respectively;
(xii) SEQ ID NOs: 355, 356, 357, and 358, TLS, and SEQ ID NO: 360, respectively;
(xiii) SEQ ID NOs: 365, 366, 367, and 368, TVS, and SEQ ID NO: 370, respectively;
(xiv) SEQ ID NOs: 375, 376, 377, and 378, GAS, and SEQ ID NO: 380, respectively;
(xv) SEQ ID NOs: 385, 386, 387, and 388, KVS, and SEQ ID NO: 390, respectively;
(xvi) SEQ ID NOs: 395, 396, 377, and 398, SAS, and SEQ ID NO: 400, respectively;
(xvii) SEQ ID NOs: 405, 406, 407, and 408, AAS, and SEQ ID NO: 410, respectively;
(xviii) SEQ ID NOs: 415, 416, 417, and 418, QDN, and SEQ ID NO: 420, respectively;
(xix) SEQ ID NOs: 425, 426, 427, and 428, AAS, and SEQ ID NO: 430, respectively;
(xx) SEQ ID NOs: 435, 436, 437, and 438, ASS, and SEQ ID NO: 440, respectively;
(xxi) SEQ ID NOs: 445, 446, 447, and 448, AAS, and SEQ ID NO: 450, respectively;
(xxii) SEQ ID NOs: 455, 456, 457, and 458, DNN, and SEQ ID NO: 460, respectively;
(xxiii) SEQ ID NOs: 465, 466, 467, and 468, GAS, and SEQ ID NO: 470, respectively;
(xxiv) SEQ ID NOs: 475, 476, 477, and 478, QDR, and SEQ ID NO: 480, respectively;
(xxv) SEQ ID NOs: 485, 486, 487, and 488, QDT, and SEQ ID NO: 490, respectively;
(xxvi) SEQ ID NOs: 495, 496, 497, and 498, DDS, and SEQ ID NO: 500, respectively;
(xxvii) SEQ ID NOs: 505, 506, 507, and 508, QDR, and SEQ ID NO: 510, respectively;
(xxviii) SEQ ID NOs: 515, 516, 517, and 518, QDN, and SEQ ID NO: 520, respectively;
(xxix) SEQ ID NOs: 525, 526, 527, and 528, EVS, and SEQ ID NO: 530, respectively;
(xxx) SEQ ID NOs: 535, 536, 537, and 538, QDT, and SEQ ID NO: 540, respectively; or
(xxxi) SEQ ID NOs: 545, 546, 547, and 548, QDR, and SEQ ID NO: 550, respectively.

In other embodiments, an antibody or antigen binding fragment thereof of the application competes with an antibody or antigen binding fragment exemplified herein. The binding site of an antibody or antigen can be determined by known methods such as ELISA, Western blot, etc. In certain embodiments, such a competing antibody binds to the same epitope (e.g., a linear or a conformational epitope) that is bound by an exemplified antibody or antigen binding fragment thereof. Detailed exemplary methods for mapping an epitope to which an antibody binds are provided in Morris, G. E., (ed.), “Epitope Mapping Protocols,” In: Methods in Molecular Biology, Vol. 66, Humana Press, Totowa, N.J. (1996). An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

Preferably, the antibody or antigen-binding fragment thereof is single-chain variable fragment (scFv) comprising the heavy chain variable region (H_{V}) covalently linked to the light chain variable region (L_{V}) via a flexible linker. The scFv can retain the specificity of the original immunoglobulin, despite removal of the constant regions and the introduction of the linker. In a scFv, the order of the domains can be either H_{V}-linker-L_{V}, or L_{V}-linker-H_{V}. The linker can be designed de novo or derived from known protein structure to provide a compatible length and conformational in bridging the variable domains of a scFv without serious steric interference. The linker can have 10 to about 25 amino acids in length. Preferably, the linker is a peptide linker spanning about 3.5 nm (35 Å) between the carboxy terminus of the variable domain and the amino terminus of the other domain without affecting the ability of the domains to fold and form an intact antigen-binding site (Huston et al., Methods in Enzymology, vol. 203, pp. 46-88, 1991, which is incorporated herein by reference in its entirety). The linker preferably comprises a hydrophilic sequence in order to avoid intercalation of the peptide within or between the variable domains throughout the protein folding (Argos, Journal of Molecular Biology, vol. 211, no. 4, pp. 943-958, 1990). For example, the linker can comprise Gly and Ser residues and/or together with the charged residues such as Glu, Thr and Lys interspersed to enhance the solubility. In one embodiment, the linker has the amino acid sequence of SEQ ID NO: 314 (GTEGKSSGSGSESKST). Any other suitable linker can also be used in view of the present disclosure.

In some embodiments, the scFv comprises an amino acid sequence having at least 80%, such as at least 85%, 90%, 91%, 92%, 93%, 94%, 95%, 96%, 97%, 98%, 99% or 100%, sequence identity to the amino acid sequences of SEQ ID NO: 278, 291, 28, 56, 84, 109, 134, 162, 190, 218, 243, 262, 344, 354, 364, 374, 384, 394, 404, 414, 424, 434, 444, 454, 464, 474, 484, 494, 504, 514, 524, 534 or 544.

In a preferred embodiment, an antibody or antigen binding fragment thereof that binds to TfR, preferably human TfR1, does not contain a free cysteine.

An anti-TfR antibody or antigen-binding fragment thereof (such as a VHH or scFv fragment) can be produced using suitable methods in the art in view of the present disclosure. For example, a VHH or scFv fragment can be recombinantly produced by growing a recombinant host cell (such as a bacterial, yeast or mammalian cell) under suitable conditions for the production of the antibody fragment and recovering the fragment from the cell culture.

### Brain Shuttle Construct

An optimized RMT brain delivery platform is developed using the transferrin receptor (TfR) by enhancing the intrinsic transcytosis efficiency, extending peripheral pharmacokinetics, and engineering for an acceptable safety profile while maintaining efficacy of the therapeutic mAb. The interplay between transcytosis receptor affinity and brain concentration in human TfR knock-in mice is studied. A thorough study of binding kinetics demonstrate that for optimal brain PK and PD of mAbs, a neutral off-rate that is neither too fast nor too slow is required. The enhanced brain delivery observed in mice was confirmed in cynomolgus monkey.

It is also discovered that engineered antibody constant region with increased binding to the neonatal Fc receptor (FcRn) resulted in decreased peripheral clearance and enhancement in brain concentration.

Additional Fc mutations are introduced to abolish binding to Fc gamma receptors (FcγR) and avoid effector function mediated toxicity. When coupled with a high affinity anti-Tau binding mAb, these mutations prevent effector function mediated toxicity in the periphery while maintaining antibody dependent phagocytosis (ADP) through a novel, non-FcγR mechanism for microglial uptake and target degradation. This mechanism is dependent upon internalization through the TfR receptor and is more efficient in promoting target degradation than traditional FcγR mediated ADP without the stimulating the secretion of pro-inflammatory cytokines. To the knowledge of the inventors, this is the first report of non-FcγR mediated ADP, representing a novel, efficient, non-inflammatory mechanism for phagocytosis that can be exploited for a variety of therapeutic applications.

Accordingly, in one general aspect, the application relates to an antibody-targeted brain delivery system comprising an anti-TfR antibody or antigen binding fragment thereof of the application. The anti-TfR antibody or antigen binding fragment thereof can be used to deliver a therapeutic or diagnostic agent into a cell (e.g., a cancer cell) or a BBB system. Agents that can be delivered include any neurological disorder drug or agent that can be used to detect or analyze a neurological disorder drug. For example, such agent can be neurotrophic factors, including, but not limited to, nerve growth factor (NGF), brain derived neurotrophic factor (BDNF), ciliary neurotrophic factor (CNTF), glial cell-line neurotrophic factor (GDNF) and insulin-like growth factor (IGF); neuropeptides, including, but not limited to, Substance P, neuropeptideY, vasoactive intestinal peptide (VIP), gamma-amino-butyric acid (GABA), dopamine, cholecystokinin (CCK), endorphins, enkephalins and thyrotropin releasing hormone (TRH); cytokines; anxiolytic agents; anticonvulsants; polynucleotides and transgenes, including, for example, small interfering RNAs and/or antisense oligos; or antibodies or antigen binding fragments thereof that bind to a brain target. An anti-hTfR antibody or antigen binding fragment thereof of the application can be an effective means to enhance the delivery of an agent of interest from the blood into the brain and function there.

In particular, an agent of interest can be delivered in a combined form or linked to an anti-TfR antibody or antigen binding fragment thereof of the application, parenterally, e.g., intravenously. For example, the agent can be non-covalently attached to the anti-TfR antibody or antigen binding fragment thereof. The agent can also be covalently attached to the anti-TfR antibody or antigen binding fragment thereof to form a conjugate. In certain embodiments, the conjugation is by construction of a protein fusion (i.e., by genetic fusion of the two genes encoding an anti-TfR antibody or antigen binding fragment thereof and a neurological disorder drug and expression as a single protein). Known methods can be used to link an agent to an antibody or antigen binding fragment thereof in view of the present disclosure. See, for example, Wu et al., Nat Biotechnol., 23(9):1137-46, 2005; Trail et al., Cancer Immunol Immunother., 52(5):328-37, 2003; Saito et al., Adv Drug Deliv Rev., 55(2):199-215, 2003; Jones et al., Pharmaceutical Research, 24(9):1759-1771, 2007.

In some embodiments, a therapeutic or diagnostic agent to be delivered to the brain and an anti-TfR antibody or antigen binding fragment thereof can be covalently linked together (or conjugated) via a non-peptide linker or a peptide linker. Examples of non-peptide linkers include, but are not limited to, polyethylene glycol, polypropylene glycol, copolymer of ethylene glycol and propylene glycol, polyoxyethylated polyol, polyvinyl alcohol, polysaccharides, dextran, polyvinyl ether, biodegradable polymer, polymerized lipid, chitins, and hyaluronic acid, or derivatives thereof, or combinations thereof. A peptide linker can be a peptide chain consisting of 1 to 50 amino acids linked by peptide bonds or a derivative thereof, whose N-terminus and C-terminus can be covalently linked to an anti-TfR antibody or an antigen binding fragment thereof.

In certain embodiments, a conjugate of the application is a multi-specific antibody comprising a first antigen binding region which binds a TfR and a second antigen binding region which binds a brain antigen, such as beta-secretase 1 (BACE1), tau, and the other brain antigens disclosed herein. Techniques for making multi-specific antibodies include, but are not limited to, recombinant co-expression of two immunoglobulin heavy chain-light chain pairs having different specificities (see Milstein and Cuello, Nature 305: 537, 1983), WO 93/08829, and Traunecker et al, EMBO J. 10: 3655, 1991), and “knob-in-hole” engineering (see, e.g., U.S. Pat. No. 5,731,168). Multi-specific antibodies can also be made by engineering electrostatic steering effects (WO 2009/089004A1); cross-linking two or more antibodies or fragments (see, e.g., U.S. Pat. No. 4,676,980, and Brennan et al, Science, 229: 81, 1985); using leucine zippers (see, e.g., Kostelny et al, J. Immunol., 148(5): 1547-1553, 1992)); using “diabody” technology (see, e.g., Hollinger et al, Proc. Natl. Acad. Sci. USA, 90:6444-6448, 1993)); using single-chain Fv (sFv) dimers (see, e.g. Gruber et al, J. Immunol, 152:5368 (1994)); and preparing trispecific antibodies as described, e.g., in Tutt et al. J. Immunol. 147: 60, 1991. A multi-specific antibody of the application also encompasses antibodies having three or more functional antigen binding sites, including “Octopus antibodies” or “dual-variable domain immunoglobulins” (DVDs) (see, e.g. US 2006/0025576A1, and Wu et al. Nature Biotechnology, 25(11):1290-7, 2007). A multi-specific antibody of the application also encompasses a “Dual Acting Fab” or “DAF” comprising an antigen binding region that binds to TfR as well as the brain antigen (e.g. BACE1 or Tau) (see, US 2008/0069820, for example). In one embodiment, the antibody is an antibody fragment, various such fragments being disclosed herein.

In one embodiment, a multi-specific antibody of the application is a fusion construct comprising an anti-TfR antibody or antigen-binding fragment thereof of the application covalently linked (or fused) to a second antibody or antigen binding fragment thereof. Preferably, the second antibody or antigen binding fragment thereof binds to a brain target, such as BACE, tau or other brain antigens, such as those described herein. The anti-TfR antibody or antigen-binding fragment thereof can be fused to the carboxy- and/or amino-terminus of a light and/or heavy chain of the second antibody or antigen binding fragment thereof, directly or via a linker.

In one embodiment, the anti-TfR antibody or antigen-binding fragment thereof is fused to the carboxy-terminus of a light chain of the second antibody or antigen binding fragment thereof, directly or via a linker.

In another embodiment, the anti-TfR antibody or antigen-binding fragment thereof is fused to the amino-terminus of a light chain of the second antibody or antigen binding fragment thereof, directly or via a linker.

In another embodiment, the anti-TfR antibody or antigen-binding fragment thereof is fused to the carboxy-terminus of a heavy chain of the second antibody or antigen binding fragment thereof, directly or via a linker.

In another embodiment, the anti-TfR antibody or antigen-binding fragment thereof is fused to the amino-terminus of a heavy chain of the second antibody or antigen binding fragment thereof, directly or via a linker.

In a preferred embodiment, a fusion construct of the application comprises an anti-TfR antibody or antigen-binding fragment thereof, preferably an anti-huTfR1 VHH or scFv fragment, of the application covalently linked, via a linker, to the carboxy terminus of only one of the two heavy chains of a second antibody or antigen binding fragment thereof that binds to a brain target. Preferably, the linker has the amino acid sequence of SEQ ID NO: 312 or SEQ ID NO: 313.

To facilitate the formation of a heterodimer between the two heavy chains, e.g., one with a fusion of the anti-TfR antibody or antigen-binding fragment thereof and one without, or one containing the Fc for the anti-TfR arm and one for the anti-brain target arm, heterodimeric mutations introduced into the Fc of the two heavy chains. Examples of such Fc mutations include, but are not limited to, the Zymework mutations (see, e.g., U.S. Pat. No. 10,457,742) and the “knob in hole” mutations (see, e.g., Ridgway et al., Protein Eng., 9(7): 617-621, 1996). Other heterodimer mutations can also be used in the invention. In some embodiment, a modified CH3 as described herein is used to facilitate the formation of a heterodimer between the two heavy chains.

In addition to the heterodimeric mutations, other mutations can also be introduced. In some embodiment, the Fc region of the fusion construct or bispecific antibody further comprises one or more mutations that alter (increase or diminish), preferably eliminate ADCC/CDC (such as the AAS mutations described herein), and/or one or more mutations that alter (increase or diminish), preferably increase, the binding of the fusion construct or bispecific antibody to FcRn (such as the YTE mutations described herein). In some embodiment, one or more cysteine residues in the fusion construct or bispecific antibody are substituted with other amino acids, such as serine.

In certain embodiments, a fusion construct of the application comprises:
(1) a first heavy chain having an amino acid sequence that is at least 80%, such as at least 85%, 90%, 95% or 100%, identical to an amino acid sequence selected from the group consisting of SEQ ID NOs: 301, 304, 307, 285, 288, 298, 10, 13, 16, 19, 22, 25, 35, 38, 41, 44, 47, 50, 53, 63, 66, 69, 72, 75, 78, 81, 91, 94, 97, 100, 103, 106, 116, 119, 122, 125, 128, 131, 141, 144, 147, 150, 153, 156, 159, 169, 172, 175, 178, 181, 184, 187, 197, 200, 203, 206, 209, 212, 215, 225, 228, 231, 234, 237, 240, 250, 252, 256, 259, 269, 272, 275, 320, 327, 334, 341, 351, 361, 371, 381, 391, 401, 411, 421, 431, 441, 451, 461 and 471;
(2) two light chains each independently having an amino acid sequence that is at least 80%, such as at least 85%, 90%, 95% or 100%, identical to an amino acid sequence selected from the group consisting of 302, 305, 308, 286, 289, 299, 11, 14, 17, 20, 23, 26, 36, 39, 42, 45, 48, 51, 54, 64, 67, 70, 73, 76, 79, 82, 92, 95, 98, 101, 104, 107, 117, 120, 123, 126, 129, 132, 142, 145, 148, 151, 154, 157, 160, 170, 173, 176, 179, 182, 185, 188, 198, 201, 204, 207, 210, 213, 216, 226, 229, 232, 235, 238, 241, 251, 253, 257, 260, 270, 273 276, 321, 328, 335, 342, 352, 362, 372, 382, 392, 402, 412, 422, 432, 442, 452, 462 and 472, respectively; and
(3) a second heavy chain having an amino acid sequence that is at least 80%, such as at least 85%, 90%, 95% or 100%, identical to an amino acid sequence selected from the group consisting of 303, 306, 309, 287, 290, 300, 12, 15, 18, 21, 24, 27, 37, 40, 43, 46, 49, 52, 55, 65, 68, 71, 74, 77, 80, 83, 93, 96, 99, 102, 105, 108, 118, 121, 124, 127, 130, 133, 143, 146, 149, 152, 155, 158, 161, 171, 174, 177, 180, 183, 186, 189, 199, 202, 205, 208, 211, 214, 217, 227, 230, 233, 236, 239, 242, 252, 254, 258, 261, 271, 274, 277, 322, 329, 336, 343, 353, 363, 373, 383, 393, 403, 413, 423, 433, 443, 453, 463 and 473, respectively.

A conjugate, such as a multi-specific antibody or fusion construct, of the application can be produced by any of a number of techniques known in the art in view of the present disclosure. For example, it can be expressed from a recombinant host cells, wherein expression vector(s) encoding the heavy and light chains of the fusion construct or multi-specific antibody is (are) transfected into a host cell by standard techniques. The host cells can be prokaryotic or eukaryotic host cells.

In an exemplary system, one or more recombinant expression vectors encoding the heterodimeric two heavy chains and the light chains of a fusion construct of the application is/are introduced into host cells by transfection or electroporation. The selected transformant host cells are cultured to allow for expression of the heavy and light chains under conditions sufficient to produce the fusion construct, and the fusion construct is recovered from the culture medium. Standard molecular biology techniques are used to prepare the recombinant expression vector, transfect the host cells, select for transformants, culture the host cells and recover the protein construct from the culture medium.

The application provides an isolated nucleic acid encoding the amino acid sequence of an anti-TfR antibody or antigen binding fragment thereof alone or as part of a fusion construct or multispecific antibody in any of the embodiments described herein or any of the claims. The isolated nucleic acid can be part of a vector, preferably an expression vector.

In another aspect, the application relates to a host cell transformed with the vector disclosed herein. In an embodiment, the host cell is a prokaryotic cell, for example, E. coli. In another embodiment, the host cell is a eukaryotic cell, for example, a protist cell, an animal cell, a plant cell, or a fungal cell. In an embodiment, the host cell is a mammalian cell including, but not limited to, CHO, COS, NS0, SP2, PER.C6, or a fungal cell, such as Saccharomyces cerevisiae, or an insect cell, such as Sf9.

### Pharmaceutical Composition and Related Methods

The invention also relates to pharmaceutical compositions, methods of preparation and methods for use thereof.

In another general aspect, the invention relates to a pharmaceutical composition, comprising an anti-TfR antibody or antigen binding fragment thereof or a conjugate thereof of the invention and a pharmaceutically acceptable carrier. The anti-TfR antibody or antigen binding fragment thereof or conjugate (such as a multi-specific antibody or fusion construct) of the invention is also useful in the manufacture of a medicament for therapeutic applications mentioned herein. The pharmaceutically acceptable carrier can be any suitable excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application.

Accordingly, in one embodiment, the application relates to a method of transporting a therapeutic or diagnostic agent across the blood-brain barrier (BBB) comprising exposing an anti-TfR antibody or antigen binding fragment thereof coupled to the therapeutic or diagnostic agent to the blood-brain barrier such that the antibody or antigen binding fragment thereof transports the agent coupled thereto across the blood-brain barrier. In one embodiment, the agent is a neurological disorder drug. In another embodiment, the agent is an imaging agent or an agent for detecting a neurological disorder. Preferably, the anti-TfR antibody or antigen binding fragment thereof or conjugate thereof does not impair the binding of the TfR to its native ligand transferrin. The antibody specifically binds to TfR in such a manner that it does not inhibit binding of the TfR to transferrin. In some embodiment, the BBB is in a mammal, preferably a primate, such as a human, more preferably a human having a neurological disorder. In one embodiment, the neurological disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

In one embodiment, an anti-TfR antibody or antigen binding fragment thereof, or a conjugate thereof of the application, is used to detect a neurological disorder before the onset of symptoms and/or to assess the severity or duration of the disease or disorder. The antibody, antigen binding fragment or conjugate thereof permits detection and/or imaging of the neurological disorder, including imaging by radiography, tomography, or magnetic resonance imaging (MRI).

In another embodiment, an anti-TfR antibody or antigen binding fragment thereof, or a conjugate thereof, is used in treating a neurological disorder (e.g., Alzheimer's disease), comprising administering to a subject in need of the treatment an effective amount of anti-TfR antibody or antigen binding fragment thereof, or a conjugate thereof. In some embodiments, the method further comprises administering to the subject an effective amount of at least one additional therapeutic agent.

In another embodiment, the application relates to the use of an anti-TfR antibody or antigen binding fragment or conjugate thereof of the application in the manufacture or preparation of a medicament. In one embodiment, the medicament is for treatment of neurological disease or disorder. In a further embodiment, the medicament is for use in a method of treating neurological disease or disorder comprising administering to an individual having neurological disease or disorder an effective amount of the medicament.

Another general aspect of the application relates to a method of inducing antibody dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, the antigen-binding fragment thereof of an anti-TfR antibody binding fragment according to an embodiment of the application, wherein the therapeutic antibody or antigen binding fragment thereof does not have effector function. For example, the therapeutic antibody or antigen binding fragment thereof can comprise one or more amino acid modifications that reduces or eliminates the effector function, such as the ADCC or CDC, such as mutations that reduce or abolish the binding to Fc gamma receptor. Such mutations can be at positions L234, L235, D270, N297, E318, K320, K322, P331, and P329, such as one, two or three mutations of L234A, L235A and P331S, wherein the numbering of amino acid residues is according to the EU index as set forth in Kabat. In one embodiment, the therapeutic antibody or antigen binding fragment thereof binds specifically to tau aggregates.

In some embodiments, the method further comprises administering to the subject an effective amount of at least one additional therapeutic agent. In certain embodiments, an additional therapeutic agent is a therapeutic agent effective to treat the same or a different neurological disorder as the anti-TfR antibody or antigen binding fragment or conjugate thereof is being employed to treat. Exemplary additional therapeutic agents include, but are not limited to: the various neurological drugs described above, cholinesterase inhibitors (such as donepezil, galantamine, rovastigmine, and tacrine), NMDA receptor antagonists (such as memantine), amyloid beta peptide aggregation inhibitors, antioxidants, γ-secretase modulators, nerve growth factor (NGF) mimics or NGF gene therapy, PPARγ agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the neurological drug. The additional therapeutic agent can be administered in the same or separate formulations and administered together or separately with the anti-TfR antibody or antigen binding fragment or conjugate thereof. The anti-TfR antibody or antigen binding fragment or conjugate of the application can be administered prior to, simultaneously with, and/or following, the administration of the additional therapeutic agent and/or adjuvant. The anti-TfR antibody or antigen binding fragment or conjugate thereof of the application can also be used in combination with other interventional therapies such as, but not limited to, radiation therapy, behavioral therapy, or other therapies known in the art and appropriate for the neurological disorder to be treated or prevented.

The anti-TfR antibody or antigen binding fragment or conjugate thereof of the application (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration, depending in part on whether the administration is brief or chronic. Various dosing schedules including but not limited to single or multiple administrations over various time-points, bolus administration, and pulse infusion are contemplated herein.

For the prevention or treatment of a disease, the appropriate dosage of an anti-TfR antibody or antigen binding fragment or conjugate thereof of the application (when used alone or in combination with one or more other additional therapeutic agents) will depend on various factors, such as the type of disease to be treated, the type of antibody or conjugate, the severity and course of the disease, whether the antibody, antigen binding fragment or conjugate thereof is administered for preventive or therapeutic purposes, previous therapy, the patient's clinical history and response to the antibody, the physiological state of the subject (including, e.g., age, body weight, health), and the discretion of the attending physician. Treatment dosages are optimally titrated to optimize safety and efficacy. The antibody, antigen binding fragment or conjugate thereof is suitably administered to the patient at one time or over a series of treatments.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

In another aspect, the application relates to an article of manufacture (such as a kit) containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers can be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody, antigen binding fragment thereof or a conjugate of the application. The label or package insert indicates that the composition is used for treating the condition of choice. Moreover, the article of manufacture can include (a) a first container with a composition contained therein, wherein the composition comprises an antibody, antigen binding fragment thereof or a conjugate of the application; and (b) a second container with a composition contained therein, wherein the composition comprises a further cytotoxic or otherwise therapeutic agent. The article of manufacture in this embodiment of the invention can further include a package insert indicating that the compositions can be used to treat a particular condition. Optionally, the article of manufacture can further comprise a second (or third) container comprising a pharmaceutically acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

### EMBODIMENTS

The invention provides also the following non-limiting embodiments.

1. An anti-TfR antibody or antigen-binding fragment thereof for delivering an agent to the brain of a subject in need thereof, wherein the anti-TfR antibody or antigen-binding fragment thereof binds to a transferrin receptor (TfR), preferably human TfR1, with a dissociation constant KD of at least 1 nM at a neutral pH and an off-rate constant k_{d} of at least 10⁻⁴ sec⁻¹ at an acidic pH, preferably the pH 5.

1a. The anti-TfR antibody or antigen-binding fragment thereof of embodiment 1, having a dissociation constant KD of 1 nM to 500 nM, such as 1 nM, 10 nM, 50 nM, 100 nM, 200 nM, 300 nM, 400 nM, 500 nM, or any value in between, at the neutral pH.

1b. The anti-TfR antibody or antigen-binding fragment thereof of embodiment 1 or 1a, having an off-rate constant k_{d} of 10⁻⁴ sec⁻¹ to 10⁻¹ sec⁻¹, such as 10⁻⁴, 10⁻³, 10⁻², 10⁻¹ sec⁻¹ or any value in between, at the acidic pH.

2. The anti-TfR antibody or antigen-binding fragment thereof of any one of embodiments 1 to 1b, having an off-rate constant k_{d} of 2×10⁻² to 2×10⁻⁴ sec⁻¹, preferably 2.0×10⁻³ sec⁻¹, at the neutral pH.

2a. The anti-TfR antibody or antigen-binding fragment thereof of embodiment 2, wherein the off-rate constant k_{d} at the neutral pH is 2×10⁻² to 2×10⁻⁴ sec⁻¹, such as 2×10⁻², 1×10⁻², 9×10⁻³, 8×10⁻³, 7×10⁻³, 6×10⁻³, 5×10⁻³, 4×10⁻³, 3×10⁻³, 2×10⁻³, 1×10⁻³, 9×10⁻⁴, 8×10⁻⁴, 7×10⁻⁴, 6×10⁻⁴, 5×10⁻⁴, 4×10⁻⁴, 3×10⁻⁴, 2×10⁻⁴ sec⁻¹, or any value in between.

3. The anti-TfR antibody or antigen-binding fragment thereof of any one of embodiments 1 to 2a, comprising:
(1) a heavy chain variable region comprising heavy chain complementarity determining regions (HCDRs) HCDR1, HCDR2 and HCDR3, and a light chain variable region comprising light chain complementarity determining regions (LCDRs) LCDR1, LCDR2 and LCDR3, wherein the HCDR1, HCDR2, HCDR3, LCDR1, LCDR2 and LCDR3 have the amino acid sequences of:
i. SEQ ID NOs: 292, 293, 294, 295, 296, and 297, respectively;
ii. SEQ ID NOs: 279, 280, 281, 282, 283 and 284, respectively;
iii. SEQ ID NOs: 29, 30, 31, 32, 33 and 34, respectively;
iv. SEQ ID NOs: 57, 58, 59, 60, 61 and 62, respectively;
v. SEQ ID NOs: 85, 86, 87, 88, 89 and 90, respectively;
vi. SEQ ID NOs: 110, 111, 112, 113, 114 and 115, respectively;
vii. SEQ ID NOs: 135, 136, 137, 138, 139 and 140, respectively;
viii. SEQ ID NOs: 191, 192, 193, 194, 195 and 196, respectively;
ix. SEQ ID NOs: 244, 245, 246, 247, 248 and 249, respectively;
x. SEQ ID NOs: 263, 264, 265, 266, 267 and 268, respectively;
xi. SEQ ID NOs: 345, 346, 347, and 348, WAS, and SEQ ID NO: 350, respectively;
xii. SEQ ID NOs: 355, 356, 357, and 358, TLS, and SEQ ID NO: 360, respectively;
xiii. SEQ ID NOs: 365, 366, 367, and 368, TVS, and SEQ ID NO: 370, respectively;
xiv. SEQ ID NOs: 375, 376, 377, and 378, GAS, and SEQ ID NO: 380, respectively;
xv. SEQ ID NOs: 385, 386, 387, and 388, KVS, and SEQ ID NO: 390, respectively;
xvi. SEQ ID NOs: 395, 396, 377, and 398, SAS, and SEQ ID NO: 400, respectively;
xvii. SEQ ID NOs: 405, 406, 407, and 408, AAS, and SEQ ID NO: 410, respectively;
xviii. SEQ ID NOs: 415, 416, 417, and 418, QDN, and SEQ ID NO: 420, respectively;
xix. SEQ ID NOs: 425, 426, 427, and 428, AAS, and SEQ ID NO: 430, respectively;
xx. SEQ ID NOs: 435, 436, 437, and 438, ASS, and SEQ ID NO: 440, respectively;
xxi. SEQ ID NOs: 445, 446, 447, and 448, AAS, and SEQ ID NO: 450, respectively;
xxii. SEQ ID NOs: 455, 456, 457, and 458, DNN, and SEQ ID NO: 460, respectively;
xxiii. SEQ ID NOs: 465, 466, 467, and 468, GAS, and SEQ ID NO: 470, respectively;
xxiv. SEQ ID NOs: 475, 476, 477, and 478, QDR, and SEQ ID NO: 480, respectively;
xxv. SEQ ID NOs: 485, 486, 487, and 488, QDT, and SEQ ID NO: 490, respectively;
xxvi. SEQ ID NOs: 495, 496, 497, and 498, DDS, and SEQ ID NO: 500, respectively;
xxvii. SEQ ID NOs: 505, 506, 507, and 508, QDR, and SEQ ID NO: 510, respectively;
xxviii. SEQ ID NOs: 515, 516, 517, and 518, QDN, and SEQ ID NO: 520, respectively;
xxix. SEQ ID NOs: 525, 526, 527, and 528, EVS, and SEQ ID NO: 530, respectively;
xxx. SEQ ID NOs: 535, 536, 537, and 538, QDT, and SEQ ID NO: 540, respectively; or
xxxi. SEQ ID NOs: 545, 546, 547, and 548, QDR, and SEQ ID NO: 550, respectively; or
(2) a single variable domain on a heavy chain (VHH) comprising heavy chain complementarity determining regions (HCDRs) HCDR1, HCDR2 and HCDR3 having the amino acid sequences of:
i. SEQ ID NOs: 7, 8 and 9, respectively;
ii. SEQ ID NOs: 317, 318 and 319, respectively;
iii. SEQ ID NOs: 324, 325 and 326, respectively;
iv. SEQ ID NOs: 331, 332 and 333, respectively; or
v. SEQ ID NOs: 338, 339 and 340, respectively

4. The antibody or antigen-binding fragment thereof of embodiment 3, being a VHH fragment comprising an amino acid sequence having at least 80%, such as at least 85%, 90%, 95% or 100%, sequence identity to SEQ ID NO: 6, 316, 323, 330, or 337.

4a. The antibody or antigen-binding fragment thereof of embodiment 2, wherein the VHH fragment comprises the amino acid sequence of SEQ ID NO: 6, 316, 323, 330, or 337.

5. The antibody or antigen-binding fragment thereof of embodiment 3, being a single-chain variable fragment (scFv) comprising the heavy chain variable region (VH) covalently linked to the light chain variable region (VL) via a linker, such as a peptide linker having about 10 to about 25 amino acids in length.

5a. The antibody or antigen-binding fragment thereof of embodiment 5, wherein the VH is linked to the amino-terminus of the VL via the linker in the scFv.

5b. The antibody or antigen-binding fragment thereof of embodiment 5, wherein the VH is linked to the carboxy-terminus of the VL via the linker in the scFv.

5c. The antibody or antigen-binding fragment thereof of embodiment 5a or 5b, wherein the linker comprises one or more of Gly and Ser, with one or more interspersed Glu, Thr and Lys residues, preferably the linker has the amino acid sequence of SEQ ID NO: 314.

5d. The antibody or antigen-binding fragment thereof of embodiment 5c, wherein the scFv comprises the HCDR1, HCDR2, HCDR3, LCDR1, LCDR2 and LCDR3 having the amino acid sequences of SEQ ID NOs: 279, 280, 281, 282, 283 and 284, respectively, or SEQ ID NOs: 292, 293, 294, 295, 296, and 297, respectively;

5e. The antibody or antigen-binding fragment thereof of embodiment 5, wherein the scFv comprises an amino acid sequence having at least 80%, such as at least 85%, 90%, 95% or 100%, sequence identity to the amino acid sequences of SEQ ID NO: 278, 291, 28, 56, 84, 109, 134, 162, 190, 218, 243, 262, 344, 354, 364, 374, 384, 394, 404, 414, 424, 434, 444, 454, 464, 474, 484, 494, 504, 514, 524, 534 or 544.

5f. The antibody or antigen-binding fragment thereof of embodiment 5e, wherein the scFv comprises the amino acid sequence of SEQ ID NO: 278, 291, 28, 56, 84, 109, 134, 162, 190, 218, 243, 262, 344, 354, 364, 374, 384, 394, 404, 414, 424, 434, 444, 454, 464, 474, 484, 494, 504, 514, 524, 534 or 544.

5g. The antibody or antigen-binding fragment thereof of embodiment 5e, wherein the scFv comprises the amino acid sequence of SEQ ID NO: 278, 291, 162 or 218.

5h. An antibody or antigen-binding fragment thereof that binds to the same epitope of the antibody or antigen-binding fragment thereof of any one of embodiments 3 to 5g.

5i. An antibody or antigen-binding fragment thereof that competes with the antibody or antigen-binding fragment thereof of any one of embodiments 3 to 5g in binding to the TfR.

5j. The antibody or antigen-binding fragment thereof of any one of embodiments 3 to 5i, binding to human TfR1 with a dissociation constant KD of 1 to 500 nM, such as 1 nM, 10 nM, 50 nM, 100 nM, 200 nM, 300 nM, 400 nM, 500 nM, or any value in between, at pH7.4.

5k. The antibody or antigen-binding fragment thereof of any one of embodiments 3 to 5j, binding to human TfR1 with an off-rate constant k_{d} of 10⁻⁴ to 10⁻¹ sec⁻¹, such as 10⁻⁴, 10⁻³, 10⁻², 10⁻¹ sec⁻¹ or any value in between, at pH5.

6. A complex comprising the antibody or antigen-binding fragment thereof of any one of embodiments 1 to 5k coupled to a therapeutic or diagnostic agent.

6a. The complex of embodiment 6, wherein the antibody or antigen-binding fragment thereof is coupled to the therapeutic or diagnostic agent noncovalently.

6b. The complex of embodiment 6, wherein the antibody or antigen-binding fragment thereof is coupled to the therapeutic or diagnostic agent covalently to form a conjugate.

6c. The complex of embodiment 6, wherein the antibody or antigen-binding fragment thereof is covalently linked to the therapeutic or diagnostic agent via a linker.

6d. The complex of embodiment 6c, wherein the linker is a non-peptide linker, such as polyethylene glycol, polypropylene glycol, copolymer of ethylene glycol and propylene glycol, polyoxyethylated polyol, polyvinyl alcohol, polysaccharides, dextran, polyvinyl ether, biodegradable polymer, polymerized lipid, chitins, and hyaluronic acid, or derivatives thereof, or combinations thereof.

6e. The complex of embodiment 6c, wherein the linker is a peptide linker, such as a peptide chain consisting of 1 to 50 amino acids linked by peptide bonds or a derivative thereof.

6f. The complex of any one of embodiments 6 to 6e, wherein the antibody or antigen-binding fragment thereof is coupled to the diagnostic agent for detecting a neurological disorder, preferably, the diagnostic agent is an agent for positron emission tomography (PET), or an agent for IDK.

6g. The complex of any one of embodiments 6 to 6e, wherein the antibody or antigen-binding fragment thereof is coupled to a therapeutic agent, preferably a neurological disorder drug.

6h. The complex of embodiment 6g, wherein the neurological disorder drug is selected from the group consisting of small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and active fragments of the foregoing.

6i. The complex of embodiment 6g, wherein the neurological disorder drug is selected from the group consisting of antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, Glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

7. The complex of embodiment 6, being a multi-specific antibody comprising a first antigen binding region which binds a TfR and a second antigen binding region which binds a brain antigen (or brain target), wherein the first antigen binding region comprises the antigen-binding fragment thereof of any one of embodiments 1 to 5k.

7a. The multi-specific antibody of embodiment 7, wherein the brain target is selected from the group consisting of beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

7b. The multi-specific antibody of embodiment 7a, wherein the second antigen binding region binds a BACE1 or Tau.

7c. The multi-specific antibody of any one of embodiments 7 to 7b, wherein the first antigen binding region is covalently linked to a first Fc, and the second antigen binding region is covalently linked to a second Fc.

7d. The multi-specific antibody of embodiment 7c, wherein the first Fc is different from the second Fc in one or more amino acid residues to facilitate the formation of a heterodimer between the first Fc and the second Fc.

8. The multi-specific antibody of any one of embodiments 7 to 7d, being a fusion construct comprising the antibody or antigen-binding fragment thereof of any one of embodiments 1 to 5k covalently linked a second antibody or antigen binding fragment thereof that binds the brain antigen (or brain target).

8a. The fusion construct of embodiment 8, wherein the antibody or antigen-binding fragment thereof of any one of embodiments 1 to 5k is covalently linked, preferably via a linker, to the amino-terminus of a heavy chain of the second antibody or antigen binding fragment thereof.

8b. The fusion construct of embodiment 8, wherein the antibody or antigen-binding fragment thereof of any one of embodiments 1 to 5k is covalently linked, preferably via a linker, to the amino-terminus of a light chain of the second antibody or antigen binding fragment thereof.

8c. The fusion construct of embodiment 8, wherein the antibody or antigen-binding fragment thereof of any one of embodiments 1 to 5k is covalently linked, preferably via a linker, to the carboxy-terminus of a light chain of the second antibody or antigen binding fragment thereof.

8d. The fusion construct of embodiment 8, wherein the antibody or antigen-binding fragment thereof of any one of embodiments 1 to 5k is covalently linked, preferably via a linker, to the carboxy-terminus of a heavy chain of the second antibody or antigen binding fragment thereof.

9. The fusion construct of embodiment 8d, wherein the antibody or antigen-binding fragment thereof any one of embodiments 1 to 5k is covalently linked, via a linker, to the carboxy terminus of only one of the two heavy chains of the second antibody or antigen binding fragment.

9a. The fusion construct of any one of embodiments 8a to 9, wherein the linker is a peptide linker comprising one or more of Gly and Ser, preferably the linker has the amino acid sequence of SEQ ID NO: 312 or SEQ ID NO: 313.

9b. The fusion construct of any one of embodiments 8 to 9a, wherein the second antibody or antigen binding fragment thereof comprise a first Fc in its first heavy chain and a second Fc in its second heavy chain, and the first Fc is different from the second Fc in one or more amino acid residues to facilitate the formation of a heterodimer between the first Fc and the second Fc.

9c. The multi-specific antibody of embodiment 7d or the fusion construct of embodiment 9b, wherein the first Fc contains one or more “knob” mutations and the second Fc contains more or more corresponding “hole” mutations, or vice versa (see, e.g., U.S. Pat. No. 5,731,168, Ridgway et al., Protein Eng., 9(7): 617-621, 1996, for the “knob-in-hole” mutations, which are incorporated herein by reference in its entirety), preferably a Knob mutation of T366W and a hole mutation of T366S, L368A or Y407V.

9d. The multi-specific antibody of embodiment 7d or the fusion construct of embodiment 9b, wherein each of the first Fc and the second Fc comprises a modified heterodimeric CH3 domain as compared to a wild-type CH3 domain polypeptide, preferably, the modified heterodimeric CH3 domain comprises one or more mutations as described in U.S. Pat. No. 10,457,742.

9e. The multi-specific antibody or fusion construct of embodiment 9d, wherein the modified heterodimeric CH3 domain of the first Fc comprises amino acid modifications at positions T350, L351, F405, and Y407, and the modified heterodimeric CH3 domain of the second Fc comprises amino acid modifications at positions T350, T366, K392 and T394.

9f. The multi-specific antibody or fusion construct of embodiment 9e, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W.

9g. The multi-specific antibody or fusion construct of embodiment 9e, wherein the modified heterodimeric CH3 domain of the first Fc comprises mutations T350V, L351Y, F405A and Y407V, and the modified heterodimeric CH3 domain of the second Fc comprises mutations T350V, T366L, K392L and T394W, or vice versa.

10. The multi-specific antibody or fusion construct of any one of embodiments 7-9g, wherein the Fc region of the multi-specific antibody or fusion construct further comprises substitutions that alter (increase or diminish), preferably increase, the binding of the second antibody or antigen binding fragment thereof to neonatal Fc receptor (FcRn).

10a. The multi-specific antibody or fusion construct of embodiment 10, wherein the second antibody or antigen binding fragment thereof comprises one or more mutations in the Fc domain that enhance binding of the fusion to the neonatal Fc receptor (RcRn).

10b. The multi-specific antibody or fusion construct of embodiment 10 or 10a, wherein the one or more mutations enhance the binding at an acidic pH.

10c. The multi-specific antibody or fusion construct of embodiment 10b, wherein the Fc of the second antibody has the M252Y/S254T/T256E (YTE) mutations, wherein the numbering of amino acid residues is according to the EU index as set forth in Kabat.

11. The multi-specific antibody or fusion construct of any one of embodiments 7-10c, wherein the Fc region of the multi-specific antibody or fusion construct further comprises substitutions that alter (increase or diminish), preferably reduces or eliminates the effector function.

11a. The multi-specific antibody or fusion construct of embodiment 11, wherein the second antibody or antigen binding fragment thereof comprises one or more mutations in the Fc domain that reduce or eliminate the effector function, such antibody dependent cellular cytotoxicity (ADCC) or complement dependent cytotoxicity (CDC).

11b. The multi-specific antibody or fusion construct of embodiment 11a, wherein the Fc of the second antibody has one or more amino acid modifications at positions L234, L235, D270, N297, E318, K320, K322, P331, and P329, wherein the numbering of amino acid residues is according to the EU index as set forth in Kabat.

11c. The multi-specific antibody or fusion construct of embodiment 11b, wherein the Fc of the second antibody has one, two or three mutations of L234A, L235A and P331S (the AAS mutations).

12. The multi-specific antibody or fusion construct of any one of embodiments 7 to 11c, wherein the first antigen binding region or the antibody or antigen-binding fragment thereof does not contain cysteine.

13. The multi-specific antibody or fusion construct of embodiments 7 to 12, wherein the second antigen binding region or the second antibody or antigen binding fragment thereof binds Tau, preferably comprising the HCDR1, HCDR2, HCDR3, LCDR1, LCDR2 and LCDR3 having the amino acid sequences of SEQ ID Nos: 554 to 559, respectively, preferably, the second antibody is a monoclonal antibody comprising a heavy chain having the amino acid sequence of SEQ ID NO: 310 and a light chain having the amino acid sequence of SEQ ID NO: 311.

14. The fusion construct of embodiment 9, comprising:
(1) a first heavy chain having an amino acid sequence that is at least 80%, such as at least 85%, 90%, 95% or 100%, identical to an amino acid sequence selected from the group consisting of SEQ ID NOs: 301, 304, 307, 285, 288, 298, 10, 13, 16, 19, 22, 25, 35, 38, 41, 44, 47, 50, 53, 63, 66, 69, 72, 75, 78, 81, 91, 94, 97, 100, 103, 106, 116, 119, 122, 125, 128, 131, 141, 144, 147, 150, 153, 156, 159, 169, 172, 175, 178, 181, 184, 187, 197, 200, 203, 206, 209, 212, 215, 225, 228, 231, 234, 237, 240, 250, 252, 256, 259, 269, 272, 275, 320, 327, 334, 341, 351, 361, 371, 381, 391, 401, 411, 421, 431, 441, 451, 461 and 471;
(2) two light chains each independently having an amino acid sequence that is at least 80%, such as at least 85%, 90%, 95% or 100%, identical to an amino acid sequence selected from the group consisting of 302, 305, 308, 286, 289, 299, 11, 14, 17, 20, 23, 26, 36, 39, 42, 45, 48, 51, 54, 64, 67, 70, 73, 76, 79, 82, 92, 95, 98, 101, 104, 107, 117, 120, 123, 126, 129, 132, 142, 145, 148, 151, 154, 157, 160, 170, 173, 176, 179, 182, 185, 188, 198, 201, 204, 207, 210, 213, 216, 226, 229, 232, 235, 238, 241, 251, 253, 257, 260, 270, 273 276, 321, 328, 335, 342, 352, 362, 372, 382, 392, 402, 412, 422, 432, 442, 452, 462 and 472, respectively; and
(3) a second heavy chain having an amino acid sequence that is at least 80%, such as at least 85%, 90%, 95% or 100%, identical to an amino acid sequence selected from the group consisting of 303, 306, 309, 287, 290, 300, 12, 15, 18, 21, 24, 27, 37, 40, 43, 46, 49, 52, 55, 65, 68, 71, 74, 77, 80, 83, 93, 96, 99, 102, 105, 108, 118, 121, 124, 127, 130, 133, 143, 146, 149, 152, 155, 158, 161, 171, 174, 177, 180, 183, 186, 189, 199, 202, 205, 208, 211, 214, 217, 227, 230, 233, 236, 239, 242, 252, 254, 258, 261, 271, 274, 277, 322, 329, 336, 343, 353, 363, 373, 383, 393, 403, 413, 423, 433, 443, 453, 463 and 473, respectively.

14a. The fusion construct of embodiment 14, wherein the two light chains have an identical amino acid sequence.

14b. The fusion construct of embodiment 14, wherein the two light chains have different amino acid sequences.

14c. The fusion construct of embodiment 14, wherein:
(1) the first heavy chain has the amino acid sequence selected from the group consisting of SEQ ID NOs: 301, 304, 307, 285, 288, 298, 10, 13, 16, 19, 22, 25, 35, 38, 41, 44, 47, 50, 53, 63, 66, 69, 72, 75, 78, 81, 91, 94, 97, 100, 103, 106, 116, 119, 122, 125, 128, 131, 141, 144, 147, 150, 153, 156, 159, 169, 172, 175, 178, 181, 184, 187, 197, 200, 203, 206, 209, 212, 215, 225, 228, 231, 234, 237, 240, 250, 252, 256, 259, 269, 272, 275, 320, 327, 334, 341, 351, 361, 371, 381, 391, 401, 411, 421, 431, 441, 451, 461 and 471;
(2) the two light chains each have the amino acid sequence selected from the group consisting of 302, 305, 308, 286, 289, 299, 11, 14, 17, 20, 23, 26, 36, 39, 42, 45, 48, 51, 54, 64, 67, 70, 73, 76, 79, 82, 92, 95, 98, 101, 104, 107, 117, 120, 123, 126, 129, 132, 142, 145, 148, 151, 154, 157, 160, 170, 173, 176, 179, 182, 185, 188, 198, 201, 204, 207, 210, 213, 216, 226, 229, 232, 235, 238, 241, 251, 253, 257, 260, 270, 273 276, 321, 328, 335, 342, 352, 362, 372, 382, 392, 402, 412, 422, 432, 442, 452, 462 and 472, respectively; and
(3) the second heavy chain has the amino acid sequence selected from the group consisting of 303, 306, 309, 287, 290, 300, 12, 15, 18, 21, 24, 27, 37, 40, 43, 46, 49, 52, 55, 65, 68, 71, 74, 77, 80, 83, 93, 96, 99, 102, 105, 108, 118, 121, 124, 127, 130, 133, 143, 146, 149, 152, 155, 158, 161, 171, 174, 177, 180, 183, 186, 189, 199, 202, 205, 208, 211, 214, 217, 227, 230, 233, 236, 239, 242, 252, 254, 258, 261, 271, 274, 277, 322, 329, 336, 343, 353, 363, 373, 383, 393, 403, 413, 423, 433, 443, 453, 463 and 473, respectively.

14d. The fusion construct of embodiment 14, wherein:
(1) the first heavy chain has the amino acid sequence of SEQ ID NOs: 285, 288, 298, or 301;
(2) the two light chains each have the amino acid sequence of 286, 289, 299 or 302, respectively; and
(3) the second heavy chain has the amino acid sequence of 287, 290, 300 or 303, respectively.

15. An isolated nucleic acid encoding the antibody or antigen-binding fragment of any one of embodiments 1-5k or the fusion construct of any one of embodiments 7-14d.

16. A vector comprising the isolated nucleic acid of claim 15.

17. A host cell comprising the nucleic acid of embodiment 15 or the vector of embodiment 16.

18. A method of producing the antibody or antigen-binding fragment of any one of embodiments 1-5k or the fusion construct of any one of embodiments 7-14d, comprising culturing a cell comprising a nucleic acid encoding the antibody or antigen-binding fragment or the fusion construct under conditions to produce the antibody or antigen-binding fragment the fusion construct, and recovering the antibody or antigen-binding fragment, the conjugate or the fusion construct from the cell or cell culture.

19. A pharmaceutical composition comprising the antibody or antigen-binding fragment of any one of embodiments 1-5k, the complex of any one of embodiments 6-6i, or the multi-specific antibody or fusion construct of any one of embodiments 7-14d, and a pharmaceutically acceptable carrier.

20. A method of treating or detecting a neurological disorder in a subject in need thereof, comprising administering to the subject an effective amount of the antibody or antigen-binding fragment of any one of embodiments 1-5k, the complex of any one of embodiments 6-6i, or the multi-specific antibody or fusion construct of any one of embodiments 7-14d, or the pharmaceutical composition of embodiment 19.

21. A method of increasing delivery of a therapeutic or diagnostic agent to the brain of a subject in need thereof, comprising administering to the subject a conjugate comprising the therapeutic or diagnostic agent coupled to the antibody or antigen-binding fragment thereof of any one of embodiments 1-5k.

22. A method of transporting a therapeutic or diagnostic agent across the blood-brain barrier (BBB) comprising exposing an anti-TfR antibody or antigen binding fragment thereof of any one of embodiments 1-5k coupled to the therapeutic or diagnostic agent to the blood-brain barrier such that the antibody or antigen binding fragment thereof transports the agent coupled thereto across the blood-brain barrier.

23. A method of delivering a therapeutic or diagnostic agent across the blood-brain barrier (BBB) of a subject in need thereof, comprising administering to the subject a complex comprising the therapeutic or diagnostic agent coupled to, preferably covalently conjugated to, the antibody or antigen-binding fragment thereof of any one of embodiments 1 to 5.

24. A method of inducing antibody dependent phagocytosis (ADP) without stimulating secretion of a pro-inflammatory cytokine in a subject in need thereof, comprising administering to the subject a complex comprising a therapeutic antibody or antigen binding fragment thereof coupled to, preferably covalently conjugated to, the antigen-binding fragment thereof of any one of embodiments 1 to 5, wherein the therapeutic antibody or antigen binding fragment thereof comprises one or more mutations in the Fc domain that reduce or eliminate the effector function, such antibody dependent cellular cytotoxicity (ADCC) or complement dependent cytotoxicity (CDC).

24a. The method of embodiment 23, wherein the therapeutic antibody or antigen binding fragment thereof comprises one or more amino acid modifications at positions L234, L235, D270, N297, E318, K320, K322, P331, and P329, wherein the numbering of amino acid residues is according to the EU index as set forth in Kabat.

24b. The method of embodiment 24a, wherein the therapeutic antibody or antigen binding fragment thereof comprises one, two or three mutations of L234A, L235A and P331S.

25. The method of any one of embodiments 20-24b, wherein the subject is in need of a treatment of a neurological disorder, preferably the neurological disorder is selected from the group consisting of neurodegenerative diseases (such as Lewy body disease, postpoliomyelitis syndrome, Shy-Draeger syndrome, olivopontocerebellar atrophy, Parkinson's disease, multiple system atrophy, striatonigral degeneration, spinocerebellar ataxia, spinal muscular atrophy), tauopathies (such as Alzheimer disease and supranuclear palsy), prion diseases (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, and nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), and cancer of the CNS and/or brain (such as brain metastases resulting from cancer elsewhere in the body).

26. The method of any one of embodiments 20 to 25, wherein the antibody or antigen-binding fragment thereof, the complex, the multispecific antibody, the fusion construct or the pharmaceutical composition is administered intravenously.

27. The method of any one of embodiments 21 to 26, wherein the therapeutic agent or therapeutic antibody or antigen binding fragment thereof is a neurological disorder drug.

28. The method of any one of embodiments 21 to 23, wherein the agent is an imaging agent or an agent for detecting a neurological disorder.

29. The method of any one of embodiments 20-28, wherein the anti-TfR antibody or antigen binding fragment thereof, complex or fusion thereof, does not impair the binding of the TfR to its native ligand transferrin.

30. The method of any one of embodiments 20 to 29, wherein the administration reduces Fc-mediated effector function.

31. The method of any one of embodiments 21 to 30, wherein the administration does not induce rapid reticulocyte depletion.

32. The method of embodiment 31, wherein the therapeutic antibody or antigen binding fragment thereof binds specifically to tau aggregates

33. The method of any one of embodiments 20-32, wherein the subject is a primate, such as a human, more preferably a human having a neurological disorder.

34. The method of embodiment 33, wherein the neurological disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

The following examples of the invention are to further illustrate the nature of the invention. It should be understood that the following examples do not limit the invention and the scope of the invention is to be determined by the appended claims.

### EXAMPLES

### Example 1

While the blood-brain barrier (BBB) prevents harmful substances from entering the brain and is essential for brain homeostasis, it presents a formidable obstacle for efficiently delivering drugs to the brain. Towards this end, a monoclonal antibody (mAb) brain shuttle platform was developed that penetrates the BBB and results in substantially higher brain concentrations than mAb alone.

### Antibody Generation (OMT Rats and Ablexis Mice)

OMT rats (OmniRat® from Ligand Pharmaceuticals) and Ablexis mice (Ablexis, LLC, San Diego, CA) were immunized with human (SEQ ID NO: 1), cyno (SEQ ID NO: 2) and marmoset (SEQ ID NO: 3) transferrin receptor (TfR) using repetitive immunizations multiple sites (RIMMS) protocols for 46 days (Ablexis), 49 days (OMT) or 50 days (OMT). Briefly, animals were repeatedly immunized at multiple subcutaneous sites proximal to regional draining lymph nodes. Serum titration (ELISA, enzyme-linked immunosorbent assay) was done at day 32 (OMT) or day 35 (Ablexis) and all animals show generally low to modest titers on human, cyno, and marmoset TfR, and no titers on the negative control. Lymph nodes were harvested from sera-positive rats and mice and fused to generate hybridomas.

Hybridomas were first screened by Meso Scale Discovery (MSD) or ELISA for binding to HEK293T huTfR (human transferrin receptor) expressing cells. All these hits were then tested in the confirmatory screen. In the confirmatory screen on fluorescence-activated cell sorting (FACS) MDCK-huTfR cells (Madin-Darby canine kidney cells) and pBECs (Microvascular Endothelial Cells, endogenous huTfR expression) were used and MDCK (parental) cells were used as negative cell line. After confirmatory screen, 616 TfR specific cell binders were identified (binding either/or/both huTfR expressing cells). From these 616 hits, 340 were binding on pBECS and MDCK-huTfR cells, 16 were binding on pBECS only and 260 were binding on MDCK-huTfR only.

The hybridomas that bound pBECs and MDCK-huTfR cells were then assessed for binding to rat TfR (SEQ ID NO: 4) and mouse TfR (SEQ ID NO: 5), checked for internalization in pBECs and competition with TfR. RNA lysates were prepared for those mAbs that were human, cyno and marmoset cross-reactive and internalized without competing for TfR. Antibody V-region sequencing data was obtained.

### Antibody Generation (Llama)

For the generation of single domain (VHH) antibodies against human TfR with cross reactivity to cyno, mouse and rat, two llamas were used for immunizations at Abcore (animal 1663L and 1663L) in project 452L. Antibody titers were determined by ELISA using TfR protein (1 μg/ml). Three bleeds from the two animals were tested and both animals showed good early titers.

Phage display was done at Abcore using their standard protocol. Two libraries were made: Library 1 (452L-1) from the second bleed of both animals, and Library 2 (452L-2) from the second and third bleed. Plasmid DNA from 12 random individual clones were sequenced and >80% contained VHH inserts with the correct reading frame. Both phage display libraries were screened with human TfR using standard Abcore panning procedures. Three rounds of panning with human TfR at 10 μg/ml were done. After panning, 94 individual clones were screened by phage ELISA for specific binding to protease-activated receptor 1 (Par1) N-terminal domain and non-specific binding to BSA (bovine serum albumin). Cross reactivity with cyno, mouse, and rat TfR was measured. 94 clones were selected for sequence analysis.

### Phage Antibody Generation

Phage libraries were panned against biotinylated huTfR, complexed with transferrin. The biotinylated complex was captured on streptavidin magnetic beads (Dynal) and exposed to the de novo pIX Fab libraries which were pre-incubated with transferrin protein at a final concentration of 100 nM (round 1 and 2) or 50 nM (round 3 and 4). Non-specific phages were washed away in PBS-Tween and bound phages were recovered by infection of MC1061F′ E. coli cells. Phages were amplified from these cells overnight and panning was repeated for a total of four rounds. Following four rounds of biopanning, monoclonal Fabs were screened for binding to human transferrin receptor in an ELISA. Clones that demonstrated binding to transferrin receptor were sequenced in the heavy and light chain variable regions.

Examples of TfR antibodies or antigen binding fragments of the invention are summarized in Table 1a below.

The binding affinities (KD, kon or ka and koff or kdis or kd) of the anti-TfR mAbs, as part of the tripod fusion constructs (BBBB constructs) described in more detail below, to TfR at neutral pH (7.4) and acidic pH (5) were measured using the following biolayer interferometry method. The results are shown in Table 1b below.

TABLE 1a

| Name | Sequence | HCDR1 | HCDR2 | HCDR3 | LCDR1 | LCDR2 | LCDR3 |
|---|---|---|---|---|---|---|---|
| TfR1 | SEQ ID | SEQ ID NO: 7 | SEQ ID | SEQ ID NO: 9 | | | |
| (VHH) | NO: 6 | GLTFSNYA | NO: 8 | AADQRAGSYSS | | | |
| | | | IGGSGGTW | GWYTRSSDSLY | | | |
| TfR2 | SEQ ID | SEQ ID NO: 29 | SEQ ID | SEQ ID NO: 31 | SEQ ID | SEQ ID | SEQ ID |
| | NO: 28 | GFTFRNAW | NO: 30 | TTDPSRIPVAGAFDY | NO: 32 | NO: 33 | NO: 34 |
| | | | IKRKIDGGT | | QSVSSTY | GASSRAT | QQYGSSPYT |
| TfR3 | SEQ ID | SEQ ID NO: 57 | SEQ ID | SEQ ID NO: 59 | SEQ ID | SEQ ID | SEQ ID |
| | NO: 56 | GFTFSSYNMNW | NO: 58 | AREGISAYDALNV | NO: 60 | NO: 61 | NO: 62 |
| | | | SSISSSSSYI | | SSSNIGNNY | DNNKRPS | GTWDSSLSAVV |
| TfR4 | SEQ ID | SEQ ID NO: 85 | SEQ ID | SEQ ID NO: 87 | SEQ ID | SEQ ID | SEQ ID |
| | NO: 84 | GFTFSSYAMNW | NO: 86 | AKEDYDSSGYYPFDY | NO: 88 | NO: 89 | NO: 90 |
| | | | SGISGSGVSTN | | HKLGDKF | QDRKRPS | QTWYSSTVI |
| TfR5 | SEQ ID | SEQ ID NO: 110 | SEQ ID | SEQ ID NO: 112 | SEQ ID | SEQ ID | SEQ ID |
| | NO: 109 | GFTFSSDAMHW | NO: 111 | ARDRQWLAFDY | NO: 113 | NO: 114 | NO: 115 |
| | | | AVIWYDGSNKY | | SSDVGGYNF | EVSKRPS | SYRDSNNFDVL |
| TfR6 | SEQ ID | SEQ ID NO: 135 | SEQ ID | SEQ ID NO: 137 | SEQ ID | SEQ ID | SEQ ID |
| | NO: 134 | GFTFNNYVMNW | NO: 136 | AKEDDDSTGYYPFDY | NO: 138 | NO: 139 | NO: 140 |
| | | | ISGSGGTT | | KLGDKFV | QDSKRPS | QTWDRSTVV |
| TfR7 | SEQ ID | SEQ ID NO: 163 | SEQ ID | SEQ ID NO: 165 | SEQ ID | SEQ ID | SEQ ID |
| | NO: 162 | GFTFSSYAMNW | NO: 164 | AREGYDSSGYNPFDY | NO: 166 | NO: 167 | NO: 168 |
| | | | ISGSGGHT | | KLGDKYA | QDSKRPS | QAWDSSTVV |
| TfR8 | SEQ ID | SEQ ID NO: 191 | SEQ ID | SEQ ID NO: 193 | SEQ ID | SEQ ID | SEQ ID |
| | NO: 190 | GGSISSTSYY | NO: 192 | ARHDWYGGSYGWFDP | NO: 194 | NO: 195 | NO: 196 |
| | | | IYYSGNT | | SLRSYY | AKNNRPS | NSRDSSGNHMV |
| TfR9 | SEQ ID | SEQ ID NO: 219 | SEQ ID | SEQ ID NO: 221 | SEQ ID | SEQ ID | SEQ ID |
| | NO: 218 | GGSFSGYY | NO: 220 | ARGSMDSSGFYSFDF | NO: 222 | NO: 223 | NO: 224 |
| | | | FIHSGST | | KLGDKF | QDRKRP | QTWYSSTVI |
| TfR10 | SEQ ID | SEQ ID NO: 244 | SEQ ID | SEQ ID NO: 246 | SEQ ID | SEQ ID | SEQ ID |
| | NO: 243 | GYSFTSYW | NO: 245 | ARMYKGRGHLFDY | NO: 247 | NO: 248 | NO: 249 |
| | | | IDPSDSYT | | QSVSKA | AASNRAT | QQYYRAPYT |
| TfR11 | SEQ ID | SEQ ID NO: 263 | SEQ ID | SEQ ID NO: 265 | SEQ ID | SEQ ID | SEQ ID |
| | NO: 262 | GYSFTSYW | NO: 264 | ARMYKGHGHLFDY | NO: 266 | NO: 267 | NO: 268 |
| | | | IDPSDSYT | | QSVSKA | AASNRAT | QQYYRAPYT |
| TfR12 | SEQ ID | SEQ ID NO: 279 | SEQ ID | SEQ ID NO: 281 | SEQ ID | SEQ ID | SEQ ID |
| | NO: 278 | GGSFSGYY | NO: 280 | ARGSMDSSGFYSFDF | NO: 282 | NO: 283 | NO: 284 |
| | | | FIHSGST | | KLGDKF | QDRKRP | QTWYSSTVI |
| TfR13 | SEQ ID | SEQ ID NO: 292 | SEQ ID | SEQ ID NO: 294 | SEQ ID | SEQ ID | SEQ ID |
| | NO: 291 | GFTFSSYAMNW | NO: 293 | AREGYDSSGYNPFDY | NO: 295 | NO: 296 | NO: 297 |
| | | | ISGSGGHT | | KLGDKYA | QDSKRPS | QAWDSSTVV |
| TfR14 | SEQ ID | SEQ ID NO: 317 | SEQ ID | SEQ ID NO: 319 | | | |
| (VHH) | NO: 316 | GLTFSNYA | NO: 318 | AADQRAGSYSSGWYTRS | | | |
| | | | IGGSGGTW | SDSLY | | | |
| TfR15 | SEQ ID | SEQ ID NO: 324 | SEQ ID | SEQ ID NO: 326 | | | |
| (VHH) | NO: 323 | GLTFSSYV | NO: 325 | ASDQRAGSLSSGWYSRR | | | |
| | | | INGDGKF | SYDTLY | | | |
| TfR16 | SEQ ID | SEQ ID NO: 331 | SEQ ID | SEQ ID NO: 333 | | | |
| (VHH) | NO: 330 | GFTFSSYA | NO: 332 | VSDQRPGTLSSGWYSRS | | | |
| | | | ISWSGRW | SDTLY | | | |
| TfR17 | SEQ ID | SEQ ID NO: 338 | SEQ ID | SEQ ID NO: 340 | | | |
| (VHH) | NO: 337 | VRISSANVV | NO: 339 | NYWNEGIRY | | | |
| | | | SISGGDP | | | | |
| TfR18 | SEQ ID | SEQ ID NO: 345 | SEQ ID | SEQ ID NO: 347 | SEQ ID | SEQ ID | SEQ ID |
| | NO: 344 | GFTFSSYW | NO: 346 | ARAGNWDRDTFDI | NO: 348 | NO: 349 | NO: 350 |
| | | | INNIGNSR | | QSVLYSSNNKIY | WAS | QQYYSTPYT |
| TfR19 | SEQ ID | SEQ ID NO: 355 | SEQ ID | SEQ ID NO: 357 | SEQ ID | SEQ ID | SEQ ID |
| | NO: 354 | GFTFSRYS | NO: 356 | ARDYMWKVFDY | NO: 358 | NO: 359 | NO: 360 |
| | | | ISSSSTNI | | QSLLDSDDGNIF | TLS | MQRIEFPIT |
| TfR20 | SEQ ID | SEQ ID NO: 365 | SEQ ID | SEQ ID NO: 367 | SEQ ID | SEQ ID | SEQ ID |
| | NO: 364 | GFIFSRYS | NO: 366 | AREYMWKVFDY | NO: 368 | NO: 369 | NO: 370 |
| | | | ISSSSTNI | | QSLLDSDDGNIF | TVS | MQRIEFPIT |
| TfR21 | SEQ ID | SEQ ID NO: 375 | SEQ ID | SEQ ID NO: 377 | SEQ ID | SEQ ID | SEQ ID |
| | NO: 374 | GFTFNSYD | NO: 376 | ARDRLGYYGLDV | NO: 378 | NO: 379 | NO: 380 |
| | | | IDTAGDT | | QSVSSSY | GAS | QQYDRSPIT |
| TfR22 | SEQ ID | SEQ ID NO: 385 | SEQ ID | SEQ ID NO: 387 | SEQ ID | SEQ ID | SEQ ID |
| | NO: 384 | GLTFNNHN | NO: 386 | ARDGIAAFDAFD | NO: 388 | NO: 389 | NO: 390 |
| | | | ISSSSSYK | | QSLVYSDGITY | KVS | MQGTHWPPT |
| TfR23 | SEQ ID | SEQ ID NO: 395 | SEQ ID | SEQ ID NO: 397 | SEQ ID | SEQ ID | SEQ ID |
| | NO: 394 | GFSLSTSGMS | NO: 396 | AGIPGY | NO: 398 | NO: 399 | NO: 400 |
| | | | IDWRDDK | | QNVATN | SAS | QQYNNYPFT |
| TfR24 | SEQ ID | SEQ ID NO: 405 | SEQ ID | SEQ ID NO: 407 | SEQ ID | SEQ ID | SEQ ID |
| | NO: 404 | GFTFSTYD | NO: 406 | ARDHGYTKFSDAFDF | NO: 408 | NO: 409 | NO: 410 |
| | | | IWYDGTNR | | QGISNY | AAS | LQHNSYPLT |
| TfR25 | SEQ ID | SEQ ID NO: 415 | SEQ ID | SEQ ID NO: 417 | SEQ ID | SEQ ID | SEQ ID |
| | NO: 414 | GGSISAYY | NO: 416 | ARQETDTTGYDYFDY | NO: 418 | NO: 419 | NO 420 |
| | | | IYASGTT | | KLGDKF | QDN | QTWDRSDAV |
| TfR26 | SEQ ID | SEQ ID NO: 425 | SEQ ID | SEQ ID NO: 427 | SEQ ID | SEQ ID | SEQ ID |
| | NO: 424 | GFTFSYYW | NO: 426 | AREQRWLKSYYYGMDV | NO: 428 | NO: 429 | NO: 430 |
| | | | ISSDGSST | | QGINSY | AAS | QQLNSYPLT |
| TfR27 | SEQ ID | SEQ ID NO: 435 | SEQ ID | SEQ ID NO: 437 | SEQ ID | SEQ ID | SEQ ID |
| | NO: 434 | AFRFSNFN | NO: 436 | ARQGIPAWDAFDL | NO: 438 | NO: 439 | NO: 440 |
| | | | ITSTGTYI | | QGISNY | ASS | LQHNSYPYT |
| TfR28 | SEQ ID | SEQ ID NO: 445 | SEQ ID | SEQ ID NO: 447 | SEQ ID | SEQ ID | SEQ ID |
| | NO: 444 | GFTFSSYN | NO: 446 | AREGISAYDALNV | NO: 448 | NO: 449 | NO: 450 |
| | | | ISSSSSYI | | QGISNF | AAS | LQHDSYPLT |
| TfR29 | SEQ ID | SEQ ID NO: 455 | SEQ ID | SEQ ID NO: 457 | SEQ ID | SEQ ID | SEQ ID |
| | NO: 454 | GFTFSSYN | NO: 456 | AREGISAYDALNV | NO: 458 | NO: 459 | NO: 460 |
| | | | ISSSSSYI | | SSNIGNNY | DNN | GTWDSSLSAVV |
| TfR30 | SEQ ID | SEQ ID NO: 465 | SEQ ID | SEQ ID NO: 467 | SEQ ID | SEQ ID | SEQ ID |
| | NO: 464 | GFTFSDYY | NO: 466 | AREDSDTSGYDPFDH | NO: 468 | NO: 469 | NO: 470 |
| | | | ISSSSVTI | | QSVSSN | GAS | QQYNNWPYT |
| TfR31 | SEQ ID | SEQ ID NO: 475 | SEQ ID | SEQ ID NO: 477 | SEQ ID | SEQ ID | SEQ ID |
| | NO: 474 | GFTLSDYD | NO: 476 | ARLYYDTSGYSSFDY | NO: 478 | NO: 479 | NO: 480 |
| | | | INSGGNII | | KLGDKF | QDR | QTWYSSTVI |
| TfR32 | SEQ ID | SEQ ID NO: 485 | SEQ ID | SEQ ID NO: 487 | SEQ ID | SEQ ID | SEQ ID |
| | NO: 484 | GFTFSSYA | NO: 486 | AKREDDTTGYHYFDY | NO: 488 | NO: 489 | NO: 490 |
| | | | ISAGGGST | | KLGDKF | QDT | QAWDRTTVV |
| TfR33 | SEQ ID | SEQ ID NO: 495 | SEQ ID | SEQ ID NO: 497 | SEQ ID | SEQ ID | SEQ ID |
| | NO: 494 | GYSISSSNWW | NO: 496 | ARKKGAYDYADAFDI | NO: 498 | NO: 499 | NO: 500 |
| | | | IYFSGST | | KIGSKS | DDS | QVWDSSSDHVV |
| TfR34 | SEQ ID | SEQ ID NO: 505 | SEQ ID | SEQ ID NO: 507 | SEQ ID | SEQ ID | SEQ ID |
| | NO: 504 | GDSITNSNFY | NO: 506 | ARYVAAPDYFDY | NO: 508 | NO: 509 | NO: 510 |
| | | | IFHGGNT | | NLGNKF | QDR | QAWDSSTVV |
| TfR35 | SEQ ID | SEQ ID NO: 515 | SEQ ID | SEQ ID NO: 517 | SEQ ID | SEQ ID | SEQ ID |
| | NO: 514 | GFTFSSYV | NO: 516 | AKLDYDTSGYDPFDF | NO: 518 | NO: 519 | NO: 520 |
| | | | ISGSGGNT | | KLGDKF | QDN | QAWVSSTAI |
| TfR36 | SEQ ID | SEQ ID NO: 525 | SEQ ID | SEQ ID NO: 527 | SEQ ID | SEQ ID | SEQ ID |
| | NO: 524 | GFTFSSDG | NO: 526 | ARDRQWLAFDY | NO: 528 | NO: 529 | NO: 530 |
| | | | IWYDGSNK | | SSDVGGYNF | EVS | SSYTDSNNFDVL |
| TfR37 | SEQ ID | SEQ ID NO: 535 | SEQ ID | SEQ ID NO: 537 | SEQ ID | SEQ ID | SEQ ID |
| | NO: 534 | GYTFTSYD | NO: 536 | MKMYYDTTGYHSFDS | NO: 538 | NO: 539 | NO: 540 |
| | | | MNPNSGDT | | RLGDRF | QDT | QTWVATTVV |
| TfR38 | SEQ ID | SEQ ID NO: 545 | SEQ ID | SEQ ID NO: 547 | SEQ ID | SEQ ID | SEQ ID |
| | NO: 544 | GGSISNSNYY | NO: 546 | ARYVAAPDYFDY | NO: 548 | NO: 549 | NO: 550 |
| | | | IFHGGNT | | KLGDKF | QDR | QAWDSSTVV |

TABLE 1b

| | | Neutral pH | | | Acidic pH | | |
|---|---|---|---|---|---|---|---|
| scFv | mAb | KD (M) | kon(1/Ms) | kdis(1/s) | KD (M) | kon(1/Ms) | kdis(1/s) |
| TfR14 | BBBB432 | 1.13E−08 | 1.67E+05 | 1.89E−03 | 1.44E−08 | 1.81E+05 | 2.59E−03 |
| SEQ ID: | SEQ ID: | | | | | | |
| 316 | 320, 321, 322 | | | | | | |
| TfR15 | BBBB435 | 1.52E−08 | 1.63E+05 | 2.46E−03 | 7.76E−09 | 1.83E+05 | 1.42E−03 |
| SEQ ID: | SEQ ID: | | | | | | |
| 323 | 327, 328, 329 | | | | | | |
| TfR16 | BBBB436 | 9.88E−09 | 1.50E+05 | 1.48E−03 | 6.87E−09 | 1.72E+05 | 1.18E−03 |
| SEQ ID: | SEQ ID: | | | | | | |
| 330 | 334, 335, 336 | | | | | | |
| TfR17 | BBBB439 | 3.65E−08 | 2.06E+05 | 7.51E−03 | 4.02E−08 | 2.01E+05 | 8.07E−03 |
| SEQ ID: | SEQ ID: | | | | | | |
| 337 | 341, 342, 343 | | | | | | |
| TfR18 | BBBB459 | 1.08E−09 | 1.24E+05 | 1.34E−04 | 9.44E−09 | 1.44E+05 | 1.35E−03 |
| SEQ ID: | SEQ ID: | | | | | | |
| 344 | 351, 352, 353 | | | | | | |
| TfR19 | BBBB464 | 5.29E−09 | 5.09E+04 | 2.69E−04 | 6.44E−08 | 5.29E+04 | 3.40E−03 |
| SEQ ID: | SEQ ID: | | | | | | |
| 354 | 361, 362, 363 | | | | | | |
| TfR20 | BBBB467 | 1.03E−08 | 6.95E+04 | 7.14E−04 | 9.25E−08 | 6.66E+04 | 6.16E−03 |
| SEQ ID: | SEQ ID: | | | | | | |
| 364 | 371, 372, 373 | | | | | | |
| TfR21 | BBBB476 | 6.98E−09 | 1.67E+05 | 1.17E−03 | 1.13E−08 | 1.79E+05 | 2.02E−03 |
| SEQ ID: | SEQ ID: | | | | | | |
| 374 | 381, 382, 383 | | | | | | |
| TfR22 | BBBB478 | 1.17E−09 | 1.03E+05 | 1.21E−04 | 3.58E−08 | 1.08E+05 | 3.85E−03 |
| SEQ ID: | SEQ ID: | | | | | | |
| 384 | 391, 392, 393 | | | | | | |
| TfR23 | BBBB479 | 2.68E−08 | 3.01E+04 | 8.05E−04 | 6.2E−08 | 3.94E+04 | 2.44E−03 |
| SEQ ID: | SEQ ID: | | | | | | |
| 394 | 401, 402, 403 | | | | | | |
| TfR24 | BBBB482 | 4.63E−09 | 2.68E+04 | 1.24E−04 | 2.78E−08 | 3.75E+04 | 1.04E−03 |
| SEQ ID: | SEQ ID: | | | | | | |
| 404 | 411, 412, 413 | | | | | | |
| TfR25 | BBBB486 | <1.0E−12 | 9.79E+04 | <1.0E−07 | 1.36E−08 | 1.14E+05 | 1.56E−03 |
| SEQ ID: | SEQ ID: | | | | | | |
| 414 | 421, 422, 423 | | | | | | |
| TfR26 | BBBB500 | 8.72E−09 | 6.96E+04 | 6.07E−04 | 2.62E−08 | 8.03E+04 | 2.11E−03 |
| SEQ ID: | SEQ ID: | | | | | | |
| 424 | 431, 432, 433 | | | | | | |
| TfR27 | BBBB504 | 6.96E−09 | 8.82E+04 | 6.14E−04 | 5.58E−08 | 8.51E+04 | 4.75E−03 |
| SEQ ID: | SEQ ID: | | | | | | |
| 343 | 441, 442, 443 | | | | | | |
| TfR28 | BBBB508 | 5.64E−09 | 1.09E+05 | 6.14E−04 | 5.57E−08 | 1.12E+05 | 6.24E−03 |
| SEQ ID: | SEQ ID: | | | | | | |
| 444 | 451, 452, 453 | | | | | | |
| TfR29 | BBBB509 | 6.45E−08 | 7.10E+03 | 4.58E−04 | 1.53E−05 | 3.41E+02 | 5.22E−03 |
| SEQ ID: | SEQ ID: | | | | | | |
| 454 | 63, 64, 65 | | | | | | |
| TfR30 | BBBB522 | 5.29E−08 | 5.27E+03 | 2.79E−04 | 1.66E−05 | 3.80E+02 | 6.30E−03 |
| SEQ ID: | SEQ ID: | | | | | | |
| 464 | 471, 472, 473 | | | | | | |
| TfR31 | BBBB529 | 2.11E−09 | 1.10E+05 | 2.31E−04 | 4.6E−08 | 1.09E+05 | 5.01E−03 |
| SEQ ID: | SEQ ID: | | | | | | |
| 474 | 481, 482, 483 | | | | | | |
| TfR32 | BBBB530 | 3.92E−09 | 1.21E+05 | 4.76E−04 | 2.92E−07 | 6.79E+04 | 1.98E−02 |
| SEQ ID: | SEQ ID: | | | | | | |
| 484 | 491, 492, 493 | | | | | | |
| TfR33 | BBBB532 | 5.1E−09 | 2.00E+05 | 1.02E−03 | 2.24E−08 | 2.14E+05 | 4.78E−03 |
| SEQ ID: | SEQ ID: | | | | | | |
| 494 | 501, 502, 503 | | | | | | |
| TfR34 | BBBB538 | 1.62E−08 | 2.07E+05 | 3.35E−03 | 2.96E−07 | 1.07E+05 | 3.17E−02 |
| SEQ ID: | SEQ ID: | | | | | | |
| 504 | 511, 512, 513 | | | | | | |
| TfR35 | BBBB539 | 2.76E−10 | 9.35E+04 | 2.59E−05 | 5.03E−08 | 8.92E+04 | 4.49E−03 |
| SEQ ID: | SEQ ID: | | | | | | |
| 514 | 521, 522, 523 | | | | | | |
| TfR36 | BBBB540 | 1.19E−08 | 7.15E+04 | 8.48E−04 | 1.2E−07 | 6.32E+04 | 7.56E−03 |
| SEQ ID: | SEQ ID: | | | | | | |
| 524 | 531, 532, 533 | | | | | | |
| TfR37 | BBBB546 | 1.68E−09 | 8.51E+04 | 1.43E−04 | 3.62E−08 | 9.40E+04 | 3.40E−03 |
| SEQ ID: | SEQ ID: | | | | | | |
| 534 | 541, 542, 543 | | | | | | |
| TfR38 | BBBB547 | 3.18E−09 | 1.90E+05 | 6.05E−04 | 5.98E−08 | 1.77E+05 | 1.06E−02 |
| SEQ ID: | SEQ ID: | | | | | | |
| 544 | 551, 552, 553 | | | | | | |

### Tripod Construct Design

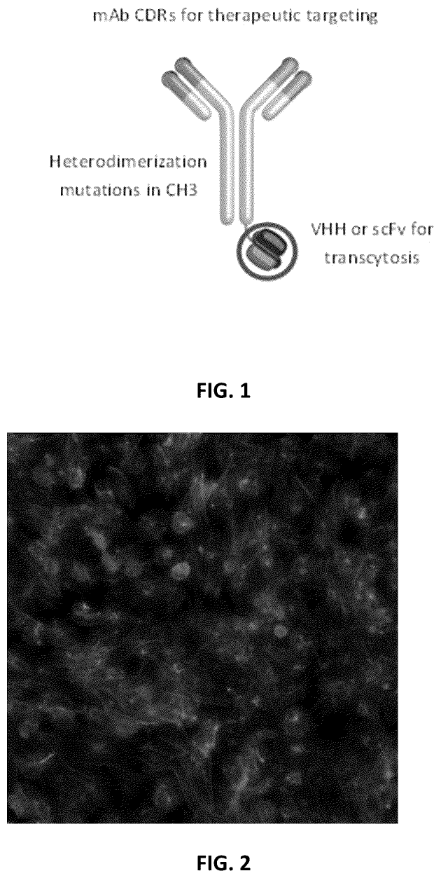

Antibodies were generated against the TfR by immunizing rodents and llamas. The resultant mAbs were screened for binding competition with transferrin and non-competitive mAbs formatted as scFv or nanobodies in a tripod mAb (also named TTP mAb) architecture and characterized. The tripod is used to deliver a substance of interest (e.g., a monoclonal antibody) to the brain. More specifically, a tripod construct (FIG. 1) containing a fusion of an antigen binding fragment of an antibody against TfR and a monoclonal antibody of interest (mAb) was developed to help the mAb penetrate the BBB and result in substantially higher brain concentrations of the mAb than the mAb alone.

For example, a tripod mAb consists of the therapeutic mAb with a TfR binding scFv or nanobody appended to the C-terminus of one antibody heavy chain using a short, flexible linker. Tripod mAbs were analysed for characteristics that have been previously described to enhance transcytosis (reviewed in Goulatis and Shusta 2017): valency, binding affinity, pH dependent binding, and rapid internalization in brain endothelial cells (FIGS. 2-4).

Heavy and light chain variable sequences of an antibody against TfR were fused in a single genetic construct as the single-chain variable fragment (scFv) using the following format, Hc_GTEGKSSGSGSESKST (SEQ ID NO: 314)_Lc. The scFv or a VHH against TfR was then fused to the C-terminus of the heavy chain (Hc) of an antibody of interest using either GGSGGS (SEQ ID NO: 312) or GGAGGA (SEQ ID NO: 313) linker. The Zymeworks heterodimerization mutations in CH3 were utilized in the antibody He (He A: T350V_L351Y_F405A_Y407V; He B: T350V_T366L_K392L_T394W) to generate the tripod construct (FIG. 1), which is also referred to as a tripod mAb. A tripod mAb contains two light chains with the identical amino acid sequence and two heavy chains with different amino acid sequences. Only one of the two heavy chains is fused to a scFv or VHH of a TfR antibody of the invention and the two heavy chains also differ in their constant regions to facilitate heterodimerization between the two heavy chains. Thus, each tripod mAb according to an embodiment of the application is associated with three amino acid sequences: the amino acid sequence of the first heavy chain fused to the antigen binding fragment of a TfR antibody, the amino acid sequence of the light chain, and the amino acid sequence of the second heavy chain not fused to the antigen binding fragment of a TfR antibody.

### Tripod Expression and Purification

Tripod mAbs were expressed in CHO-Expi cells and purified using Protein A affinity chromatography followed by Size Exclusion chromatography or Jon-exchange chromatography.

Examples of the Tripod mAbs made are provided in Table 2a:

TABLE 2a

| | mAb | scFv | VHH | | | |
|---|---|---|---|---|---|---|
| Tripod | component | component | component | VL SEQ | VH SEQ | VHH/scFv |
| name | name (target) | name | name | ID NO | ID NO | SEQ ID NO |
| BBBB434 | B21M (IgG1 | | TfR1 | 11 | 12/10 | 6 |
| | AAS) | | | | | |
| BBBB978 | BACE | | TfR1 | 14 | 13/15 | 6 |
| | (IgG1AAS) | | | | | |
| BBBB1009 | Tau (IgG1) | | TfR1 | 17 | 16/18 | 6 |
| BBBB1011 | Tan (IgG1 | | Tfr1 | 20 | 19/21 | 6 |
| | AASYTE) | | | | | |
| BBBB1073 | B-amyloid | | Tfr1 | 23 | 22/24 | 6 |
| BBBB1215 | B21M (IgG4 | | TfR1 | 26 | 25/27 | 6 |
| | PAA) | | | | | |
| BBBB501 | B21M (IgG1 | TfR2 | | 36 | 35/37 | 28 |
| | AAS) | | | | | |
| BBBB951 | B21M (IgG1) | TfR2 | | 39 | 38/40 | 28 |
| BBBB979 | BACE | TfR2 | | 42 | 41/43 | 28 |
| | (IgG1AAS) | | | | | |
| BBBB1520 | Tau (IgG1 | TfR2 | | 45 | 44/46 | 28 |
| | AASYTE) | | | | | |
| BBBB1018 | Tau (IgG1) | TfR2 | | 48 | 47/49 | 28 |
| BBBB1076 | B-amyloid | TfR2 | | 51 | 50/52 | 28 |
| BBBB1216 | B21M (IgG4 | TfR2 | | 54 | 53/55 | 28 |
| | PAA) | | | | | |
| BBBB509 | B21M (IgG1 | TfR3 | | 64 | 65/65 | 56 |
| | AAS) | | | | | |
| BBBB946 | B21M (IgG1) | TfR3 | | 67 | 66/68 | 56 |
| BBBB947 | BACE | TfR3 | | 70 | 69/71 | 56 |
| | (IgG1AAS) | | | | | |
| BBBB1023 | Tau (IgG1 | TfR3 | | 73 | 72/74 | 56 |
| | AASYTE) | | | | | |
| BBBB1021 | Tau (IgG1) | TfR3 | | 76 | 75/77 | 56 |
| BBBB1079 | B-amyloid | TfR3 | | 79 | 78/80 | 56 |
| BBBB1217 | B21M (IgG4 | TfR3 | | 82 | 81/83 | 56 |
| | PAA) | | | | | |
| BBBB520 | B21M (IgG1 | TfR4 | | 92 | 91/93 | 84 |
| | AAS) | | | | | |
| BBBB975 | BACE | TfR4 | | 95 | 94/96 | 84 |
| | (IgG1AAS) | | | | | |
| BBBB1026 | Tau (IgG1 | TfR4 | | 98 | 97/99 | 84 |
| | AASYTE) | | | | | |
| BBBB1024 | Tau (IgG1) | TfR4 | | 101 | 100/102 | 84 |
| BBBB1082 | B-amyloid | TfR4 | | 104 | 103/105 | 84 |
| BBBB1218 | B21M (IgG4 | TfR4 | | 107 | 106/108 | 84 |
| | PAA) | | | | | |
| BBBB534 | B21M (IgG1 | TfR5 | | 117 | 116/118 | 109 |
| | AAS) | | | | | |
| BBBB945 | B21M (IgG1) | TfR5 | | 120 | 119/121 | 109 |
| BBBB973 | BACE | TfR5 | | 123 | 122/124 | 109 |
| | (IgG1AAS) | | | | | |
| BBBB1035 | Tau (IgG1 | TfR5 | | 126 | 125/127 | 109 |
| | AASYTE) | | | | | |
| BBBB1033 | Tau (IgG1) | TfR5 | | 129 | 128/130 | 109 |
| BBBB1219 | B21M (IgG4 | TfR5 | | 132 | 131/133 | 109 |
| | PAA) | | | | | |
| BBBB537 | B21M (IgG1 | TfR6 | | 142 | 141/143 | 134 |
| | AAS) | | | | | |
| BBBB989 | B21M (IgG1) | TfR6 | | 145 | 144/156 | 134 |
| BBBB977 | BACE | TfR6 | | 148 | 147/149 | 134 |
| | (IgG1AAS) | | | | | |
| BBBB1038 | Tau (IgG1 | TfR6 | | 151 | 150/152 | 134 |
| | AASYTE) | | | | | |
| BBBB1036 | Tau (IgG1) | TfR6 | | 154 | 153/155 | 134 |
| BBBB1085 | B-amyloid | TfR6 | | 157 | 156/158 | 134 |
| BBBB1220 | B21M (IgG4 | TfR6 | | 160 | 159/161 | 134 |
| | PAA) | | | | | |
| BBBB543 | B21M (IgG1 | TfR7 | | 170 | 169/171 | 162 |
| | AAS) | | | | | |
| BBBB1112 | B21M (IgG1) | TfR7 | | 173 | 172/174 | 162 |
| BBBB969 | BACE | TfR7 | | 176 | 175/177 | 162 |
| | (IgG1AAS) | | | | | |
| BBBB1048 | Tau (IgG1 | TfR7 | | 179 | 178/180 | 162 |
| | AASYTE) | | | | | |
| BBBB1046 | Tau (IgG1) | TfR7 | | 182 | 181/183 | 162 |
| BBBB1088 | B-amyloid | TfR7 | | 185 | 184/186 | 162 |
| BBBB1221 | B21M (IgG4 | TfR7 | | 188 | 187/189 | 162 |
| | PAA) | | | | | |
| BBBB556 | B2 IM (IgG1 | TfR8 | | 198 | 197/199 | 190 |
| | AAS) | | | | | |
| BBBB993 | B21M (IgG1) | TfR8 | | 201 | 200/202 | 190 |
| BBBB983 | BACE | TfR8 | | 204 | 203/205 | 190 |
| | (IgG1AAS) | | | | | |
| BBBB1051 | Tau (IgG1 | TfR8 | | 207 | 206/208 | 190 |
| | AASYTE) | | | | | |
| BBBB1049 | Tau (IgG1) | TfR8 | | 210 | 209/211 | 190 |
| BBBB1091 | B-amyloid | TfR8 | | 213 | 212/214 | 190 |
| BBBB1222 | B21M (IgG4 | TfR8 | | 216 | 215/217 | 190 |
| | PAA) | | | | | |
| BBBB557 | B21M (IgG1 | TfR9 | | 226 | 225/227 | 218 |
| | AAS) | | | | | |
| BBBB970 | BACE | TfR9 | | 229 | 228/230 | 218 |
| | (IgG1AAS) | | | | | |
| BBBB1055 | Tau (IgG1 | TfR9 | | | 231/233 | 218 |
| | AASYTE) | | | | | |
| BBBB1053 | Tau (IgG1) | TfR9 | | 235 | 234/236 | 218 |
| BBBB1094 | B-amyloid | TfR9 | | 238 | 237/239 | 218 |
| BBBB1223 | B21M (IgG4 | TfR9 | | 241 | 240/242 | 218 |
| | PAA) | | | | | |
| BBBB354 | B21M (IgG1 | TfR10 | | 251 | 250/252 | 243 |
| | AAS) | | | | | |
| BBBB932 | B21M (IgG1) | TfR10 | | 254 | 253/255 | 243 |
| BBBB383 | BACE | TfR10 | | 257 | 256/258 | 243 |
| | (IgG1AAS) | | | | | |
| BBBB1224 | B21M (IgG4 | TfR10 | | 260 | 259/261 | 243 |
| | PAA) | | | | | |
| BBBB368 | B21M (IgG1 | TfR11 | | 270 | 269/271 | 262 |
| | AAS) | | | | | |
| BBBB934 | B21M (IgG1) | TfR11 | | 273 | 273/274 | 262 |
| BBBB426 | BACE | TfR11 | | 276 | 275/277 | 262 |
| | (IgG1AAS) | | | | | |
| BBBB1136 | Tau (IgG1 | TfR12 | | 286 | 285/287 | 278 |
| | AASYTE) | | | | | |
| BBBB1134 | Tau (IgG1) | TfR12 | | 289 | 288/290 | 278 |
| BBBB1133 | Tau (IgG1 | TfR13 | | 299 | 298/300 | 291 |
| | AASYTE) | | | | | |
| BBBB1131 | Tau (IgG1) | TfR13 | | 302 | 301/303 | 291 |
| BBBB1166 | B-amyloid | TfR13 | | 305 | 304/306 | 291 |
| BBBB432 | B21M (IgG1 | TfR14 | | 321 | 320/322 | 316 |
| | AAS | | | | | |
| BBBB435 | B21M (IgG1 | TfR15 | | 328 | 327/329 | 323 |
| | AAS | | | | | |
| BBBB436 | B21M (IgG1 | TfR16 | | 335 | 334/336 | 330 |
| | AAS | | | | | |
| BBBB439 | B21M (IgG1 | TfR17 | | 342 | 341/343 | 337 |
| | AAS | | | | | |
| BBBB459 | B21M (IgG1 | TfR18 | | 352 | 351/353 | 344 |
| | AAS | | | | | |
| BBBB464 | B21M (IgG1 | TfR19 | | 362 | 361/363 | 354 |
| | AAS | | | | | |
| BBBB467 | B21M (IgG1 | TfR20 | | 372 | 371/373 | 364 |
| | AAS | | | | | |
| BBBB476 | B21M (IgG1 | TfR21 | | 382 | 381/383 | 374 |
| | AAS | | | | | |
| BBBB478 | B21M (IgG1 | TfR22 | | 392 | 391/393 | 384 |
| | AAS | | | | | |
| BBBB479 | B21M (IgG1 | TfR23 | | 402 | 401/403 | 394 |
| | AAS | | | | | |
| BBBB482 | B21M (IgG1 | TfR24 | | 412 | 411/413 | 404 |
| | AAS | | | | | |
| BBBB486 | B21M (IgG1 | TfR25 | | 422 | 421/423 | 414 |
| | AAS | | | | | |
| BBBB500 | B21M (IgG1 | TfR26 | | 432 | 431/433 | 424 |
| | AAS | | | | | |
| BBBB504 | B21M (IgG1 | TfR27 | | 442 | 441/443 | 434 |
| | AAS | | | | | |
| BBBB508 | B21M (IgG1 | TfR28 | | 452 | 451/453 | |
| | AAS | | | | | |
| BBBB509 | B21M (IgG1 | TfR29 | | 462 | 461/463 | 454 |
| | AAS | | | | | |
| BBBB522 | B21M (IgG1 | TfR30 | | 472 | 471/473 | 464 |
| | AAS | | | | | |
| BBBB529 | B21M (IgG1 | TfR31 | | 482 | 481/483 | 474 |
| | AAS | | | | | |
| BBBB530 | B21M (IgG1 | TfR32 | | 492 | 491/493 | 484 |
| | AAS | | | | | |
| BBBB532 | B21M (IgG1 | TfR33 | | 502 | 501/503 | 494 |
| | AAS | | | | | |
| BBBB538 | B21M (IgG1 | TfR34 | | 512 | 511/513 | 504 |
| | AAS | | | | | |
| BBBB539 | B21M (IgG1 | TfR35 | | 522 | 521/523 | 514 |
| | AAS | | | | | |
| BBBB540 | B21M (IgG1 | TfR36 | | 532 | 531/533 | 524 |
| | AAS | | | | | |
| BBBB546 | B21M (IgG1 | TfR37 | | 542 | 541/543 | 534 |
| | AAS | | | | | |
| BBBB547 | B21M (IgG1 | TfR38 | | 552 | 551/553 | 544 |
| | AAS | | | | | |

### Cell Binding and TfR Specificity

Tripod mAbs were analysed for characteristics that have been previously described to enhance transcytosis (reviewed in Goulatis and Shusta 2017): valency, binding affinity, pH dependent binding, and rapid internalization in brain endothelial cells (FIGS. 2-4).

Human brain endothelial cells (hCMECD3, 50,00 cells) were incubated with 10 ug/mL purified tripod mAb and allowed to incubate overnight at 4° C. in either the presence or absence of 10× molar concentration of huTfR1 ECD (SEQ ID NO: 1). Cells were fixed and washed the following morning, incubated with secondary antibody (Jackson Immunosciences Cat #109-546-170), washed again and then analyzed by FACS. Positive binders were defined as having binding signal greater than 2-fold over isotype control and a ratio of binding signal/binding signal with TfR ECD≥2 (Table 2b).

TABLE 2b
hCMECD3 cell binding and specificity for tripod mAbs.

| | | Antibody Binding | | | Antibody Binding | | | |
|---|---|---|---|---|---|---|---|---|
| | | without TfR | | | with TfR | | | |
| Protein Batch ID | SEQ ID NOs: | Rep 1 | Rep 2 | Ave Signal | Rep 1 | Rep 2 | Ave Signal | Ratio |
| BBBB434 | 10, 11, 12 | 1749 | 1405 | 1577 | 808 | 834 | 821 | 1.9 |
| BBBB501 | 35, 36, 37 | 2281 | 1848 | 2065 | 507 | 790 | 649 | 3.18 |
| BBBB509 | 63, 64, 65 | 1921 | 1814 | 1868 | 632 | 626 | 629 | 2.97 |
| BBBB520 | 91, 92, 93 | 3413 | 3588 | 3501 | 766 | 874 | 820 | 4.27 |
| BBBB534 | 116, 117, 118 | 3378 | 3246 | 3312 | 782 | 849 | 816 | 4.06 |
| BBBB537 | 141, 142, 143 | 3667 | 3531 | 3599 | 841 | 803 | 822 | 4.38 |
| BBBB543 | 169, 170, 171 | 1490 | 1291 | 1391 | 776 | 738 | 757 | 1.84 |
| BBBB556 | 197, 198, 199 | 3262 | 3539 | 3401 | 711 | 749 | 730 | 4.66 |
| BBBB557 | 225, 226, 227 | 2394 | 2204 | 2299 | 327 | 361 | 344 | 6.68 |

Additional hCMECD3 cell binding assay was conducted to measure specificity for additional tripod mAbs, and the results of which are shown in Table 2c below:

TABLE 2c
IgG4 versions:

| Name | Binding Target | Description | ELN | ka (1/Ms) | kd (1/s) | KD (M) |
|---|---|---|---|---|---|---|
| BBBB1215 (n = 6) | TfR | B21M IgG4 ZW BBBB434 | BBB-00735 | (4.56 ± 0.26)E+05 | (2.83 ± 0.25)E−03 | (6.24 ± 0.75)E−09 |
| BBBB1217 (n = 3) | TfR | B21M IgG4 ZW BBBB509 | BBB-00735 | No Binding | | |
| BBBB1224 (n = 6) | TfR | B21M IgG4 ZW BBBB354 | BBB-00735 | (9.90 ± 0.96)E+05 | (1.10 ± 0.21)E−02 | (1.11 ± 0.23)E−08 |
| BBBB1227 (n = 3) | CD98hc | B21M IgG4 ZW BBBB445 | BBB-00718 | (4.40 ± 0.17)E+05 | (2.42 ± 0.64)E−03 | (5.49 ± 1.23)E−09 |
| BBBB1228 (n = 3) | CD98hc | B21M IgG4 ZW BBBB448 | BBB-00718 | (4.26 ± 1.13)E+05 | (1.77 ± 0.71)E−03 | (4.21 ± 2.34)E−09 |
| BBBB1229 (n = 3) | CD98hc | B21M IgG4 ZW BBBB449 | BBB-00718 | (4.82 ± 1.34)E+05 | (3.36 ± 2.38)E−03 | (6.88 ± 3.14)E−09 |

Results are Average ± 95% CI

### Transferrin Competition

MDCK cells expressing recombinant human transferrin receptor were plated at 10,000 cells per well in a MA6000 384 HB plate and cultured for 18 hours in DMEM media supplemented with 10% FBS and 500 μg/mL Geneticin. Prior to the assay the cells were incubated in serum-free DMEM media supplemented with 5 M Monensin for 1 h in a 37° C. CO₂ incubator and then for 30 minutes at room temperature with StartingBlock (PBS) supplemented with 5 M Monensin. The cells in alternate rows of the plate were incubated for 30 minutes at room temperature with 2.7 mg/mL human holo transferrin prepared in serum-free DMEM media supplemented with 50M Monensin. Test antibodies were diluted to 5 μg/mL in serum-free DMEM media supplemented with 5 M Monensin and added to duplicate wells containing the holo transferrin or to duplicate wells that received no transferrin and then incubated for 1 h at room temperature. The supernatants were removed and 2 μg/mL Sulfo-TAG labeled anti-human antibody was added to each well and incubated for 30 minutes at room temperature. All wells were washed with PBS and Surfactant-Free MSD Read Buffer T was added. The plates were read on an MSD SECTOR® S600 imager.

Statistical analysis including mean, standard deviation and RSD were performed in Excel. Any samples with an RSD>25% were excluded. The mean values of the test antibodies incubated in the presence of transferrin were compared against the mean values in the absence of transferrin. Antibodies with values in the presence of transferrin that were ≤70% of the values in the absence of transferrin were considered ligand competitive (Table 3).

TABLE 3
Selected tripod mAbs are not competitive with transferrin.

| | | w/o Tf | with Tf | Relative % |
|---|---|---|---|---|
| Sample ID | SEQ ID NO: | avg value | avg value | Ab binding |
| BBBB434 | 10, 11, 12 | 1758 | 1762 | 100.3 |
| BBBB501 | 35, 36, 37 | 1348 | 1618 | 120.0 |
| BBBB509 | 63, 64, 65 | 282 | 447 | 158.6 |
| BBBB520 | 91, 92, 93 | 1369 | 1798 | 131.3 |
| BBBB534 | 116, 117, 118 | 1350 | 1141 | 84.5 |
| BBBB537 | 141, 142, 143 | 1504 | 1851 | 123.1 |
| BBBB543 | 169, 170, 171 | 225 | 233 | 103.8 |
| BBBB556 | 197, 198, 199 | 1246 | 1531 | 122.9 |
| BBBB557 | 225, 226, 227 | 598 | 530 | 88.7 |

### Internalization

Human brain endothelial cells (hCMEC/D3) were plated at 10,000 cells/well in Collagen-coated 384-well Cell Carrier Ultra plates (Perkin Elmer) and allowed to attach for 16 hours at 37° C. in a humidified incubator. The cells (50,00 cells) were then incubated with 200 ug/mL purified tripod mAb and allowed to incubate at 37° C. for one hour. The cells were fixed, washed and incubated with a fluorescently labeled secondary antibody for one hour. The cells were then washed again and incubated with fluorescently labeled actin stain, Phalloidin, and nuclear stain, Hoeschst 33342. Cells were washed again and imaged using the ImageXpress Micro (Molecular Devices) with a 40× objective. Internalizing mAbs were identified on the basis of colocalization with Phalloidin using MetaXpress 6.0. All mAbs from Tables 2 and 3 were positive for internalization.

Affinity Analysis & pH-Dependent Binding, Species Cross-Reactivity

Affinity and pH dependency were initially measured using a Forte Bio Octet Platform. Biotinylated huTfR was immobilized on streptavidin sensors and mAbs associated for 180 seconds in 0.1M Phosphate pH 7.4. Dissociation was completed for 300 seconds in either 0.1M Phosphate pH 7.4 or 0.1M Phosphate pH 5 (Table 4). Preferably, a tripod mAb of interest has a high binding affinity at pH7.4 and low binding affinity at pH5, e.g., KD≥1 nM and kd≥10⁻⁴ sec⁻¹ preferably about 10⁻³ at pH 5, such that the tripod mAb binds to TfR at a neutral pH (e.g., pH7.4) and dissociates from TfR at an acidic pH (e.g., pH5). Preferably, the KD at the acidic pH and the neutral pH and are similar, such as at a ratio of KD acidic/KD neutral of about 1.5.

TABLE 4
Kinetic rate constants measured using the Octet platform to huTfR.

| | pH 7.4 Dissociation | | | pH 5 Dissociation | | | Ratio |
|---|---|---|---|---|---|---|---|
| Sample ID | KD (M) | kₒₙ (1/Ms) | k_{dis} (1/s) | KD (M) | kₒₙ (1/Ms) | k_{dis} (1/s) | pH 5/pH 7.4 |
| BBBB434 | 1.73E−08 | 1.53E+05 | 2.64E−03 | 2.45E−08 | 1.66E+05 | 4.06E−03 | 1.54 |
| BBBB501 | 1.60E−08 | 1.47E+05 | 2.35E−03 | 1.68E−08 | 1.53E+05 | 2.57E−03 | 1.09 |
| BBBB509 | 6.45E−08 | 7.10E+03 | 4.58E−04 | 1.53E−05 | 3.41E+02 | 5.22E−03 | 11.39 |
| BBBB520 | 3.28E−09 | 1.53E+05 | 5.00E−04 | 7.32E−08 | 1.39E+05 | 1.02E−02 | 20.41 |
| BBBB534 | 1.15E−08 | 7.52E+04 | 8.63E−04 | 7.14E−08 | 7.67E+04 | 5.48E−03 | 6.35 |
| BBBB537 | 3.77E−10 | 1.38E+05 | 5.22E−05 | 4.42E−08 | 1.37E+05 | 6.05E−03 | 115.81 |
| BBBB543 | 8.29E−08 | 2.63E+05 | 2.18E−02 | 1.62E−05 | 9.86E+03 | 1.60E−01 | 7.33 |
| BBBB556 | <1.0E−12 | 8.57E+04 | <1.0E−07 | 1.99E−08 | 1.07E+05 | 2.13E−03 | Faster |
| BBBB557 | 2.94E−08 | 2.28E+05 | 6.69E−03 | 2.13E−06 | 2.97E+04 | 6.33E−02 | 9.46 |

To gain additional accuracy for affinity measurements, tripod mAb affinity for huTfR was determined using surface plasmon resonance (SPR) on a BioRad Proteon instrument, ProteOn XPR36 system. An Fc capture surface was generated by coupling anti-IgG Fc mAb (Jackson ImmunoResearch) to a GLC chip (BioRad) using the amine-coupling chemistry (BioRad). Tripod mAbs were captured using a concentration of 0.3 ug/mL, flowed for 30 seconds at 60 uL/min for a target density of 120 RU huTfR was then flowed over the immobilized tripod mAbs at concentrations from 3.125-800 nM (in a 4-fold serial dilution) for 3 min (at 50 μL/min) association followed by dissociation for 10 minutes at 50 uL/min. The chip surface was regenerated with two 18 second pulses of 100 mM H₃PO₄ (Sigma) at 100 μL/min. The collected data were processed using ProteOn Manager software V3.1.0.6 (BioRad). First, the data was corrected for background using inter-spots. Then, double reference subtraction of the data was performed by using the buffer injection for analyte injections. The kinetic analysis of the data was performed using a Langmuir 1:1 binding model. The result for each mAb was reported in the format of Ka (On-rate), Kd (Off-rate) and KD (equilibrium dissociation constant) (Table 5).

TABLE 5
Binding affinity of anti-TfR brain shuttles for TfR when fused to B21M mAb or BACE mAb.

| | B21M mAb | | |
|---|---|---|---|
| mAb | ka | kd | KD |
| (B21M/BACE) | (1/Ms) | (1/s) | (M) |
| BBBB434/ | 5.39E+05 | 2.34E−03 | 4.34E−09 |
| BBBB978 | | | |
| BBBB501/ | 2.45E+05 | 2.45E−03 | 1.00E−08 |
| BBBB979 | | | |
| BBBB520/ | 6.31E+05 | 6.28E−04 | 9.95E−10 |
| BBBB975 | | | |
| BBBB534/ | 1.03E+05 | 5.50E−04 | 5.35E−09 |
| BBBB973 | | | |
| BBBB537/ | 7.34E+05 | 2.55E−04 | 3.48E−10 |
| BBBB977 | | | |
| BBBB543/ | 3.27E+05 | 1.90E−02 | 5.80E−08 |
| BBBB969 | | | |
| BBBB556/ | 1.94E+05 | 1.70E−04 | 8.78E−10 |
| BBBB983 | | | |
| BBBB557/ | 4.22E+05 | 6.43E−03 | 1.52E−08 |
| BBBB970 | | | |
| BBBB354/ | (14.4 ± 0.59)E+05 | (2.2 ± 0.19)E−02 | (1.54 ± 0.2)E−08 |
| BBBB383 | | | |
| BBBB368/ | (5.98 ± 0.27)E+05 | (6.06 ± 1.49)E−02 | (1.1 ± 10.21)E−07 |
| BBBB426 | | | |

| | BACE mAb | | |
|---|---|---|---|
| mAb | ka | kd | KD |
| (B21M/BACE) | (1/Ms) | (1/s) | (M) |
| BBBB434/ | (4.23 ± 0.14)E+05 | (2.67 ± 0.06)E−03 | (6.31 ± 0.10)E−09 |
| BBBB978 | | | |
| BBBB501/ | (2.60 ± 0.15)E+05 | (2.67 ± 0.05)E−03 | (1.03 ± 0.06)E−08 |
| BBBB979 | | | |
| BBBB520/ | (5.91 ± 0.44)E+05 | (6.59 ± 0.11)E−04 | (1.12 ± 0.09)E−09 |
| BBBB975 | | | |
| BBBB534/ | (1.08 ± 0.06)E+05 | (4.34 ± 0.35)E−04 | (4.03 ± 0.46)E−09 |
| BBBB973 | | | |
| BBBB537/ | (6.75 ± 0.50)E+05 | (1.52 ± 0.08)E−04 | (2.27 ± 0.28)E−10 |
| BBBB977 | | | |
| BBBB543/ | (2.73 ± 0.03)E+05 | (2.23 ± 0.21)E−02 | (8.16 ± 0.92)E−08 |
| BBBB969 | | | |
| BBBB556/ | (1.99 ± 0.10)E+05 | <8.67E−05 | <4.36E−10 |
| BBBB983 | | | |
| BBBB557/ | (3.45 ± 0.09)E+05 | (7.21 ± 0.30)E−03 | (2.09 ± 0.05)E−08 |
| BBBB970 | | | |
| BBBB354/ | (11.1 ± 0.29)E+05 | (2.00 ± 0.18)E−02 | (1.79 ± 0.2)E−08 |
| BBBB383 | | | |
| BBBB368/ | (8.55 ± 0.15)E+05 | (11.1 ± 0.65)E−02 | (1.3 ± 0.68)E−07 |
| BBBB426 | | | |

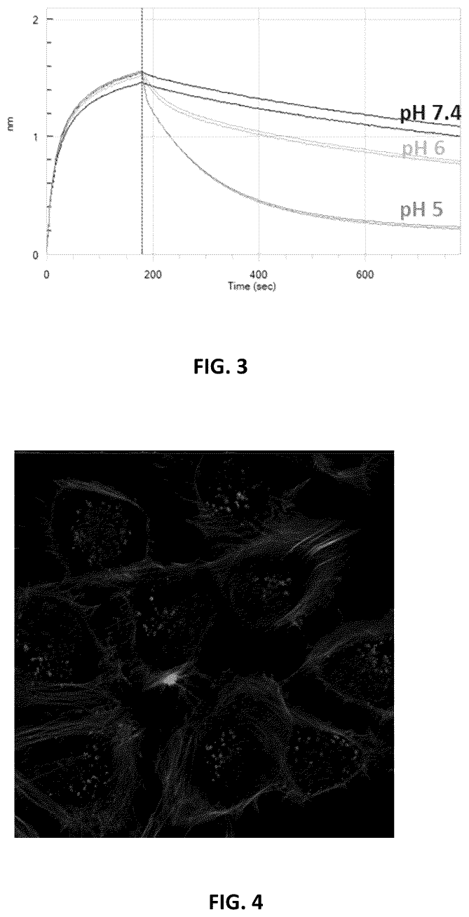

pH dependent binding was assessed using the SPR (proteon) method described above, except during the dissociation the buffer pH was stepped down from 7.4 to 6.5 to 6.0. The individual sensorgrams were evaluated and scored for pH dependent binding if the off-rate was faster as the pH decreased (example in FIG. 3).

Species cross-reactivity was assessed using the same method as for determining binding affinity, except the TfRs used were cyno (SEQ ID NO: 2), marmoset (SEQ ID NO: 3), rat (SEQ ID NO: 4) and mouse (SEQ ID NO: 5). No rat or mouse cross-reactive mAbs were identified. Cyno and marmoset cross-reactive tripod mAbs were identified (Table 6).

TABLE 6
Species cross-reactivity for selected tripod mAbs

| | Cynomolgus | | | Marmoset | | |
|---|---|---|---|---|---|---|
| mAB | ka (1/Ms) | kd (1/s) | KD (M) | ka (1/Ms) | kd (1/s) | KD (M) |
| BBBB520 | 5.29E+05 | 2.52E−04 | 4.77E−10 | 3.73E+05 | 3.66E−04 | 9.81E−10 |
| BBBB534 | 2.44E+05 | 7.78E−03 | 3.19E−08 | 2.10E+05 | 4.99E−04 | 2.38E−09 |
| BBBB537 | 6.18E+05 | 8.83E−05 | 1.43E−10 | 2.66E+05 | 6.56E−05 | 2.46E−10 |
| BBBB543 | 2.50E+05 | 2.91E−02 | 1.17E−07 | 2.04E+05 | 7.73E−03 | 3.80E−08 |
| BBBB556 | 8.68E+04 | 2.05E−03 | 2.36E−08 | 4.73E+04 | 1.04E−03 | 2.20E−08 |
| BBBB557 | 2.95E+05 | 1.86E−02 | 6.31E−08 | 1.91E+05 | 1.53E−02 | 8.03E−08 |

An anti-TfR antibody or antigen-binding fragment of the invention can be used to deliver any type of immunoglobulin. Similar results have been observed with IgG1 and IgG4 therapeutic mAbs delivered by the tripod structure (data not shown).

### Mouse Pharmacokinetics and Pharmacodynamics and Anti-BACE mAb Brain Shuttles

To analyze the impact of binding properties on transcytosis an in vivo PK/PD study was completed in mice. C57BL/6-Tfrctm2618(TFRC)Arte mice (Taconic Artemis) were administered with test articles by IV bolus injection (13 mg/kg, 10 mL/kg). At the scheduled timepoints mice were anesthetized by inhalation isoflurane. Blood collected via cardiac puncture, and plasma processed. Mouse brain was collected following whole-body perfusion with 5 mLs of 0.9% saline solution. The collected brain sample (minus cerebellum) was split into right/left hemispheres, snap frozen in liquid nitrogen, and stored at −70° C. until tissue homogenization and capillary depletion processing.

None of the TfR binding molecules were cross-reactive with murine TfR so human TfR KI mice were used to assess transcytosis. The tripod mAb was formatted with an anti-beta secretase 1 (BACE1) antagonist mAb to allow for pharmacodynamic assessment of the mAb following transcytosis into the brain. BACE1 cleaves beta-amyloid to release Aβ₁₋₄₀. The inhibition of BACE1 is measured by quantitating the concentration of the product Aβ₁₋₄₀ in the brain. Mice were dosed intravenously with two tripod mAbs, BBBB383 and BBBB426, along with the control mAb, BBBB456. BBBB456 is the anti-BACE1 antagonist mAb alone. BBBB383 and BBBB426 differ only in their affinity for TfR, K_{D}=18 nM and 130 nM respectively. Brain exposure was determined following perfusion and capillary depletion to reduce interference from mAb in blood or retained within the vascular endothelium (Johnsen, Burkhart et al. 2017). The brain concentration of both BBBB383 and BBBB426 was enhanced over BBBB456 at all timepoints, with BBBB383 having greater mAb brain concentration than BBBB426. A strong PK/PD relationship was observed with mAb brain concentration correlative with a reduction in Aβ₁₋₄₀ levels. The lower plasma exposure of both TfR containing mAbs is attributed to TMDD through binding to TfR in the periphery.

Selected anti-TfR brain shuttles were then fused to a prototypical anti-BACE (β-secretase) mAb and binding affinity was reassessed using same method as described above. As shown in Table 5, the affinity of the anti-TfR brain shuttles was similar when fused to B21M mAb (anti-human respiratory syncytial virus) and anti-BACE antagonist mAb. Internalization was assessed for selected molecules and found unchanged from internalization observed when the anti-TfR brain shuttle was fused to B21M mAb.

Since none of the anti-TfR brain shuttles bound to mouse or rat TfR, in vivo rodent studies were conducted in huTfR knock-in mice (C57BL/6-Tfrctm2618(TFRC)Arte mice (Taconic Artemis)) using the prototypical anti-BACE antagonist mAb (BBBB456, SEQ ID NOs: 307, 308 and 309). The anti-BACE antagonist mAb was selected as a model PD system for measuring inhibition of BACE1 (through the concentration of its product peptide, Aβ1-40), a reflection of the amount of mAb that was trafficked to the brain.

The first in vivo study assessed the PK/PD relationship in the brains of huTfR mice. The knock-in (KI) mice were dosed at 13 mg/kg i.v. with BBBB383 (SEQ ID NOs: 256, 257 and 258), BBBB426 (SEQ ID NOs: 275, 276 and 277) and BBBB456 (SEQ ID NO: 307, 308 and 309). Brains and plasma were harvested at 4, 24 and 72 hours. At the scheduled timepoints, mice were anesthetized by inhalation of isoflurane. Mouse brains from KI mice were collected following whole-body perfusion with 5 mL of 0.9% saline solution. The collected brain sample (minus cerebellum) was split into right/left hemispheres, snap frozen in liquid nitrogen, and stored at −70° C. until tissue homogenization and capillary depletion processing.

For sample preparation of the capillary-depleted brain tissue lysates, individual weights were obtained for the brain hemispheres to measure drug concentration. The brain tissue samples were added to a calculated volume of modified dPBS buffer (2.5-3 μL buffer per 1 mg tissue) containing protease inhibitor (Pierce; A32955) and transferred to Lysing Matrix D (MP Biomedicals™; 6913-100) tubes. Tissue samples were homogenized at 2.9 m/s for 15 seconds using a Bead Ruptor 24 Elite (Omni International). The total cell suspension was transferred into a new tube and mixed with an equal volume of a 26% dextran buffer (13% final dextran concentration). Mixed tissue homogenate was centrifuged at 2,000 g for 10 minutes at 4° C. Carefully, the upper layer (capillary-depleted fraction) was separated from the remaining sample and transferred to a new tube containing 10×RIPA lysis buffer (Millipore™; 20-188). Capillary-depleted samples plus lysis buffer were vortexed well, centrifuged at 14,000 rpm for 30 minutes at 4° C., and supernatant transferred to a new tube. Brain tissue sample lysates were either stored frozen at −70° C. or measured for protein concentrations using BCA protein assay kit (Pierce™; 23227). Final brain tissue sample lysates were normalized to 7 mg/mL total protein concentration prior to immunoassay determination of BBB-enabled mAbs.

The concentration of BBB-enabled mAbs in mouse brain tissue for PK assessment was determined using MesoScale Discovery (MSD®; Gaithersburg, MD) ECLIA technology developed in a typical sandwich immunoassay format. The assay was performed on MSD Gold™ Small Spot Streptavidin 96-well plates (Cat: L45SA). Briefly, streptavidin-coated plates were blocked with 1% bovine serum albumin (BSA) in 1× phosphate buffered saline (PBS) for 30 minutes at room temperature. The standard curve was prepared fresh in 50% naïve C57BL/6 mouse brain tissue lysates by serial dilution. Frozen quality controls (QCs) prepared in naïve C57BL/6 mouse brain tissue lysates at 2× of the working assay concentration were diluted and tested with each assay. Master mix containing the capture (biotinylated anti-human Fc mAb, 1 g/mL) and detection (ruthenium-labeled anti-human Fc mAb, 0.5 μg/mL) reagents was combined with diluted standards, QCs, and samples at a 1:1 volume ratio in the assay plate. The mixture was incubated for 1 hour with shaking at room temperature. Assay plates were washed, and MSD T read buffer (1×) was added to all wells. Raw data values were read on an MSD SECTOR® S600 imager. The standard curve range for the assay was tested at 1-512 ng/mL with a minimum required sample dilution (MRD) of 1:2, yielding a limit of sensitivity of 2 ng/mL in brain tissue lysates. The MSD output files with the raw ECL counts were imported into Watson LIMS (Thermo Scientific) and then regressed with a 5-parameter logistic fit with 1/F2 weighting.

The concentration of BBB-enabled mAbs in mouse plasma for PK assessment was determined using a similar protocol as described above. The standard curve was prepared fresh in 10% pooled mouse plasma by serial dilution. Frozen QCs prepared in pooled mouse plasma at 10× of the working assay concentration were diluted and tested with each assay. Master mix containing the capture (biotinylated anti-human Fc mAb, 1 μg/mL) and detection (ruthenium-labeled anti-human Fc mAb, 0.5 μg/mL) reagents was combined with diluted standards, QCs, and samples at a 1:1 volume ratio in the assay plate. The mixture was incubated with shaking for 1 hour at room temperature. Assay plates were washed, and MSD T read buffer (lx) was added to all wells. Raw data values were read on an MSD SECTOR® S600 imager. The standard curve range for the assay was tested at 2-512 ng/mL with an MRD of 1:10, yielding a limit of sensitivity of 20 ng/mL in plasma matrix. The MSD output files with the raw ECL counts were imported into Watson LIMS (Thermo Scientific) and the regressed with a 5-parameter logistic fit with 1/F2 weighting.

BACE activity measurements were made by homogenizing mouse brains in 2 ml lysing matrix D tube (8 μl of 0.4% DEA/50 mM NaCl per mg of brain weight, Fast Prep-24 at 6/shakes/sec for 20 sec). Tubes were then centrifuged at 4° C. for 5 min in an Eppendorf Centrifuge set to a maximum speed. Homogenate (supernatant) was then transferred to precooled tubes which were then centrifuged for 70 minutes at 13,000 rpm at 4° C. Supernatant was then transferred to a tube containing 10% of 0.5 M Tris/HCL and frozen at −80° C. until assayed. Aβ 1-40 peptide standards and thawed processed brain homogenate are pre-complexed at 1:1 with ruthenium (Meso Scale Discovery (MSD), R91AN-1) labeled anti-Aβ antibody. 50 ul of complex was added to blocked plate containing capture antibody to Aβ 1-40. After overnight incubation at 2-8° C. with no shaking, plates were washed and 2× read buffer (MSD, R92TC-1) added. Plate was read using Meso Sector S 600 (MSD, ICOAA).

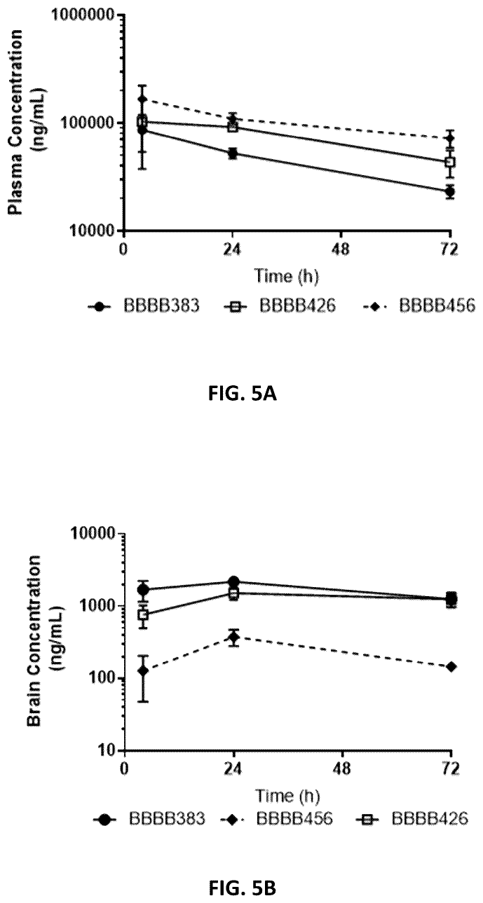

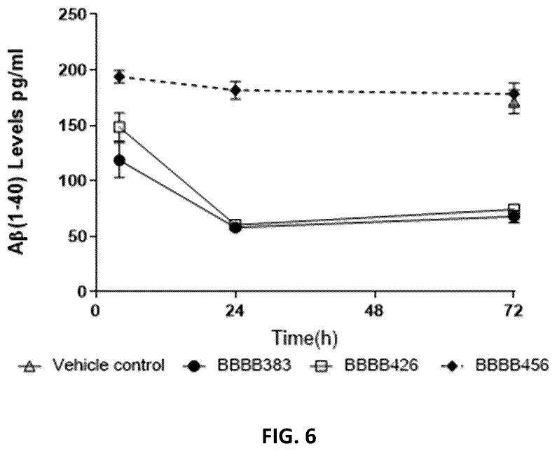

Brain shuttle containing mAbs BBBB383 and BBBB426 were observed to have faster plasma clearance than the anti-BACE mAb BBBB456 alone (FIG. 5A). However, the converse was true in the brain with BBBB383 and BBBB426 observed to have increased brain concentration at all timepoints over the control BBBB456. When the PD effect of BACE inhibition was measured, both brain shuttle mAbs were observed to inhibit the activity of BACE to a greater extent than the control anti-BACE mAb alone. (FIG. 5B).

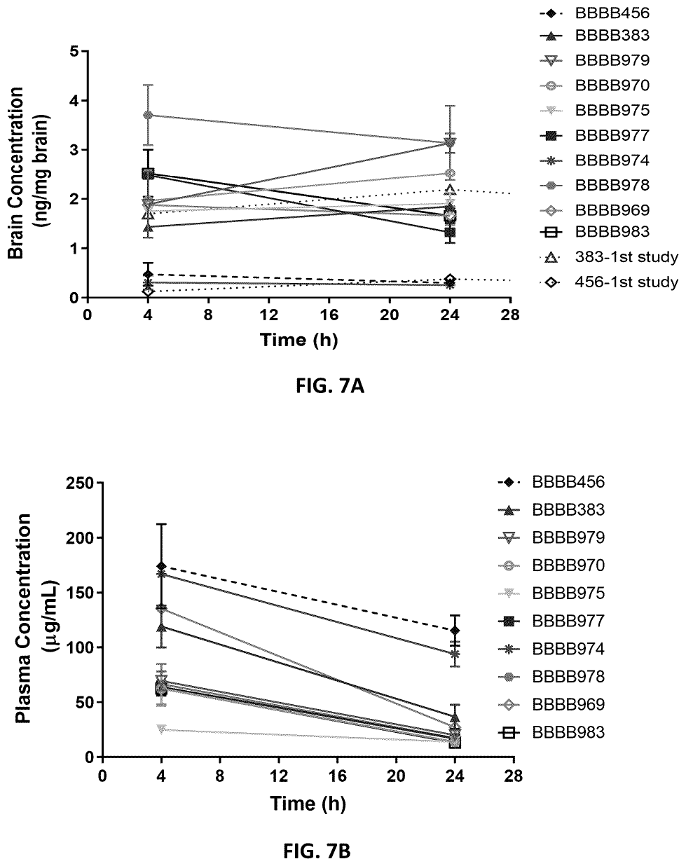

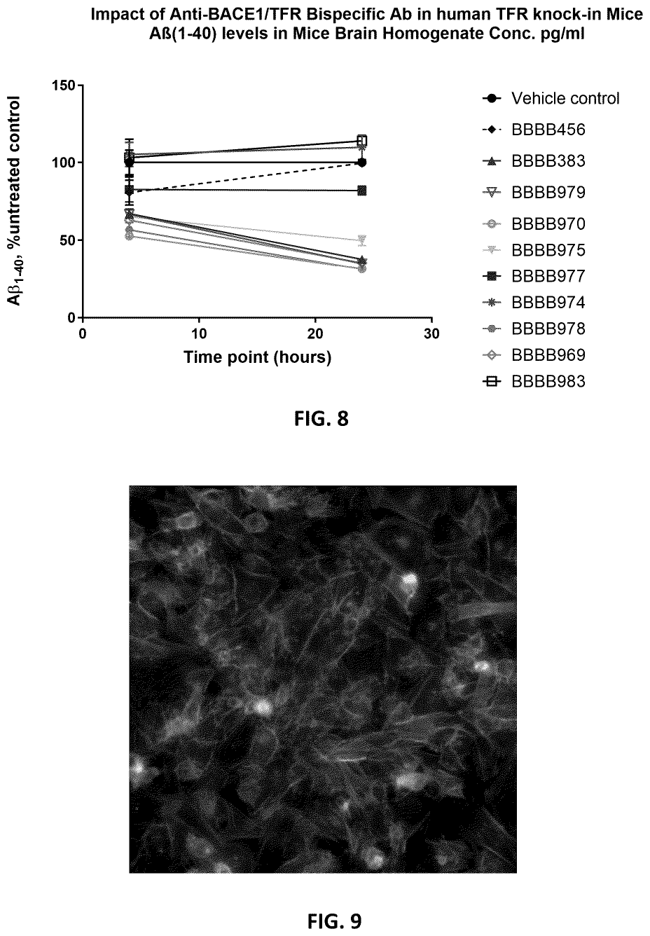

Additional brain shuttle containing mAbs were similarly assessed at 4 and 24 hrs following 13 mg/kg i.v. dosing (FIG. 7A-B). Similar to the first study, all brain shuttle mAbs were observed to have faster plasma clearance than the control anti-BACE mAb. A range of brain concentration was observed for the brain shuttle mAbs with enhancement in brain concentration for all except BBBB974. It was hypothesized that BBBB974 did not traffic efficiently to the brain due to its binding kinetics. Specifically, BBBB974 has a slow neutral on-rate that may prevent efficient association with the TfR in vivo. A tripod mAb concentration dependent decrease in Aβ 1-40 levels were also observed for all tripod mAbs except BBBB983, which had an increase in brain concentration over the control BBBB456 but no impact on Apl-40 concentration (FIG. 8). This observation may be due to the binding kinetics, as BBBB983 has a very slow neutral off-rate which may prevent the efficient diffusion in the brain that is necessary for BACE inhibition. These data underscore the importance of TfR binding kinetics for both the delivery and function of a therapeutic mAb.

The relationship between affinity and transcytosis efficiency has been described previously as improved transcytosis with decreased affinity for TfR (Yu, Zhang et al. 2011), a conclusion that is discrepant with the above data. To probe the transcytosis affinity relationship in more detail, nine tripod mAb were assessed for brain PK/PD in the mouse model described above. These tripod mAbs differed in affinity for the TfR by approximately 100-fold (K_{D} ranged from 0.2 nM-81 nM). Brain concentration was measured at 24 hours (C_{max brain}) following IV dosing (FIG. 17). As expected, a range of transcytosis efficiency was observed from no enhancement to ten-fold improvement over the control mAb. The data indicate a more nuanced relationship between affinity and transcytosis efficiency than what has been previously described, with influence from both on- and off-rates impacting brain concentration. For example, no enhancement in brain exposure for BBBB946 over the control mAb was observed, although it had a K_{D}=65 nM and a fast off-rate at pH 6. This mAb was unique though, having a slower on-rate than other mAbs under study (kₐ≈10³ M⁻¹ s⁻¹ compared with kₐ≈10⁵ M⁻¹ s⁻¹). In fact, when compared with another tripod antibody, BBBB969, with similar K_{D} (K_{D}=81 nM) but 100× faster on-rate the contrast is apparent. BBBB969 enhanced brain concentration by 5.5-fold demonstrating the importance of a sufficiently fast on-rate for efficient brain delivery. The efficiency of transcytosis for the other eight mAbs studied can be best described by their off-rates, with optimal brain delivery occurring at an off-rate that is neither too fast nor too slow (optimal neutral k_{d} of 2×10⁻³ s⁻¹). A strong PK/PD relationship was observed for all tripod mAbs except for BBBB983, which had 5.5× enhancement in brain concentration but no impact on Aβ₁₋₄₀ levels. This mAb has a slow neutral off-rate (<8×10⁻⁵ s⁻¹) which we hypothesize impacts its ability to diffuse in the brain to the target. Taken together the data demonstrate the importance of optimizing both the neutral on- and off-rates for optimal brain PK and PD. We observed no influence of binding epitope on TfR in the study (data not shown).

Selection of mAbs for Cyno Studies and Assessment of Brain Shuttles Fused to Anti-Tau mAb

Critical to confirming the ability of the TfR targeting tripod mAbs to enhance therapeutic antibody brain exposure in humans is demonstrating enhanced brain delivery in non-human primates. The best performing tripod mAbs in the mouse study (BBBB979 and BBBB978) did not bind to the cyno TfR and were therefore excluded from further study. The next best, BBBB970 and BBBB969, both contained a free cysteine residue in the light chain of the anti-TfR brain shuttle (SEQ ID NO: 162 and SEQ ID NO: 218). Since free cysteine residues can contribute to nonideal biophysical properties during manufacturing, the free cysteines were mutated to serine residues (SEQ ID NO: 278 and SEQ ID NO: 291).

The new scFvs were fused to the anti-Tau mAb, PT1B844 (SEQ ID NOs: 310 and 311) to generate BBBB1136 (SEQ ID NOs: 285, 286 and 287)/BBBB1134 (SEQ ID NOs: 288, 289 and 290), and BBBB1133 (SEQ ID NOs: 298, 299 and 300)/BBBB1131 (SEQ ID NOs: 301, 302 and 303) (IgG1 AAS YTE/IgG1). The affinity for huTfR was measured (Table 7).

TABLE 7
Binding affinity of anti-TfR brain shuttles for huTfR
with Cys-Ser mutations fused to the anti-Tau mAb

| | ka (1/Ms) | ka (1/Ms) | KD (M) |
|---|---|---|---|
| BBBB1131 | (2.53 ± 0.08)E+05 | (3.17 ± 0.05)E−02 | (1.25 ± 0.05)E−07 |
| BBBB1133 | (2.38 ± 0.04)E+05 | (3.23 ± 0.29)E−02 | (1.36 ± 0.15)E−07 |
| BBBB1134 | (2.12 ± 0.10)E+05 | (2.49 ± 0.11)E−03 | (1.18 ± 0.10)E−08 |
| BBBB1136 | (2.08 ± 0.15)E+05 | (2.26 ± 0.06)E−03 | (1.09 ± 0.11)E−08 |

BBBB1134/BBBB1136 maintained very similar binding to huTfR as BBBB557/BBBB970, indicating that neither the Cys-Ser mutation or fusion to the anti-Tau mAb perturbed the binding affinity for huTfR. However, BBBB1131/BBBB1133 was approximately 2-fold weaker in binding affinity compared with BBBB543/BBBB969. To determine if the shift in affinity was due to the cys-ser mutation or to fusion to the anti-Tau mAb, the brain shuttle without the mutation but fused to anti-Tau was generated and binding assessed (BBBB1048 (SEQ ID NOs: 178, 179 and 180)/BBBB1046 (SEQ ID NOs: 181, 182 and 183)). The affinity of the non-mutated BBBB1048/BBBB1046 was very similar to BBBB543/BBB969, indicating that the loss of affinity was due to the cys-ser mutation and not due to fusion to the Tau mAb (Table 8).

TABLE 8
Binding affinity of anti-TfR brain shuttles
for huTfR fused to the anti-Tau mAb

| | ka (l/Ms) | ka (l/Ms) | KD (M) |
|---|---|---|---|
| BBBB1046 | (2.63 ± 0.03)E+05 | (1.82 ± 0.15)E−02 | (6.91 ± 0.50)E−08 |
| BBBB1048 | (2.57 ± 0.14)E+05 | (2.06 ± 0.25)E−02 | (7.99 ± 0.71)E−08 |

Similar to previous studies, internalization was also assessed and fusion of the brain shuttle to anti-Tau mAb did not impact its ability to internalize in human brain endothelial cells (example in FIG. 9, mAbs tested are in Table 9).

TABLE 9
Anti-TfR brain shuttles assessed for internalization
in human brain endothelial cells.

| mAb | Internalized |
|---|---|
| BBBB1046 | Yes |
| BBBB1047 | Yes |
| BBBB1048 | Yes |
| BBBB1052 | Yes |
| BBBB1053 | Yes |
| BBBB1054 | Yes |
| BBBB1055 | Yes |
| BBBB1131 | Yes |
| BBBB1132 | Yes |
| BBBB1133 | Yes |
| BBBB1134 | Yes |
| BBBB1135 | Yes |
| BBBB1136 | Yes |

Cyno Pharmacokinetics of Anti-Tau Brain Shuttle mAbs

Cynomolgus monkeys were administered with test articles by IV injection (slow bolus) with the indicated dose. At the scheduled timepoints, cynomolgus monkey brain was collected and rinsed with cold saline solution following upper body perfusion with saline for minimum of 5 minutes. Predefined brain locations were isolated, snap frozen in liquid nitrogen, and stored at −80° C. until tissue homogenization and capillary depletion processing.

BBBB1133, BBBB1136 and BBBB1134 were dosed i.v. at 10 mg/kg along with the non-brain shuttle-enabled mAbs PT1B844 (FIG. 18) and PT1B916 in cynomolgus monkeys. Plasma was sampled at 4, 24 and 72 hours. Cynomolgus monkey brain was collected and rinsed with cold saline solution following upper body perfusion with saline for minimum of 5 minutes. Predefined brain locations were isolated, snap frozen in liquid nitrogen, and stored at −80° C. until tissue homogenization and capillary depletion processing.

For sample preparation of the capillary-depleted brain tissue lysates, individual tissue weights were obtained for the brain locations collected. The brain tissue samples were added to a calculated volume of modified dPBS buffer (2.5 L buffer/1 mg tissue) containing protease inhibitor (Pierce; A32955) and transferred to Lysing Matrix D (MP Biomedicals™; 6913-100) tubes. Tissue samples were homogenized at 2.9 m/s for 15 seconds using a Bead Ruptor 24 Elite (Omni International). The total cell suspension was transferred into a new tube and mixed with an equal volume of a 26% dextran buffer (13% final dextran concentration). Mixed tissue homogenate was centrifuged at 2,000 g for 10 minutes at 4° C. Carefully, the upper layer (capillary-depleted fraction) was separated from the remaining sample and transferred to a new tube containing 10×RIPA lysis buffer (Millipore™; 20-188). Capillary-depleted samples plus lysis buffer were vortexed well, centrifuged at 14,000 rpm for 30 minutes at 4° C., and supernatant transferred to a new tube. Brain tissue sample lysates were either stored frozen at −70° C. or measured for protein concentrations using BCA protein assay kit (Pierce™; 23227). Final brain tissue sample lysates were normalized to 7 mg/mL total protein concentration prior to immunoassay determination of BBB-enabled mAbs.

The concentration of BBB-enabled mAbs in cynomolgus monkey brain tissue for PK assessment was determined using MSD® ECLIA technology developed in a typical sandwich immunoassay format. The assay was performed on MSD Gold™ Small Spot Streptavidin 96-well plates. The streptavidin-coated plates were blocked with 1% bovine serum albumin (BSA) in 1× phosphate buffered saline (PBS) for 30 minutes at room temperature. The standard curve was prepared fresh in 50% naïve cyno brain tissue lysates by serial dilution. Frozen QCs prepared in naïve cyno brain tissue lysates at 2× of the working assay concentration were diluted and tested with each assay. Master mix containing the capture (biotinylated anti-human Fc mAb, 1 μg/mL) and detection (ruthenium-labeled anti-human Fc mAb, 0.5 μg/mL) reagents was combined with diluted standards, QCs, and samples at a 1:1 volume ratio in the assay plate. The mixture was incubated for 1 hour with shaking at room temperature. Assay plates were washed, and MSD T read buffer (lx) was added to all wells. Raw data values were read on an MSD SECTOR® S600 imager. The standard curve range for the assay was tested at 1-512 ng/mL with a minimum required sample dilution (MRD) of 1:2, yielding a limit of sensitivity of 2 ng/mL in brain tissue lysates. The MSD output files with the raw ECL counts were imported into Watson LIMS (Thermo Scientific) and then regressed with a 5-parameter logistic fit with 1/F2 weighting.

The concentration of BBB-enabled mAbs in cynomolgus monkey plasma for PK assessment was determined using MSD® ECLIA technology developed in a typical sandwich immunoassay format. The assay was performed on MSD Gold™ Streptavidin 96-well plates, the streptavidin-coated plates blocked with 1% bovine serum albumin (BSA)+0.5% Tween-20 in 1× phosphate buffered saline (PBS) for 30 minutes at room temperature. The standard curve was prepared fresh in 10% pooled cyno plasma by serial dilution. Frozen QCs prepared in pooled cyno plasma at 10× of the working assay concentration were diluted and tested with each assay. Master mix containing the capture (biotinylated anti-human Fc mAb, 1 μg/mL) and detection (ruthenium-labeled anti-human Fc mAb, 1 μg/mL) reagents was combined with diluted standards, QCs, and samples at a 1:1 volume ratio in the assay plate. The mixture was incubated with shaking for 1 hour at room temperature. Assay plates were washed, and MSD T read buffer (lx) was added to all wells. Raw data values were read on an MSD SECTOR® S600 imager. The standard curve range for the assay was tested at 2-512 ng/mL with a minimum required sample dilution (MRD) of 1:10, yielding a limit of sensitivity of 20 ng/mL in plasma matrix. The MSD output files with the raw ECL counts were imported into Watson LIMS (Thermo Scientific) and then regressed with a 5-parameter logistic fit with 1/y2 weighting.

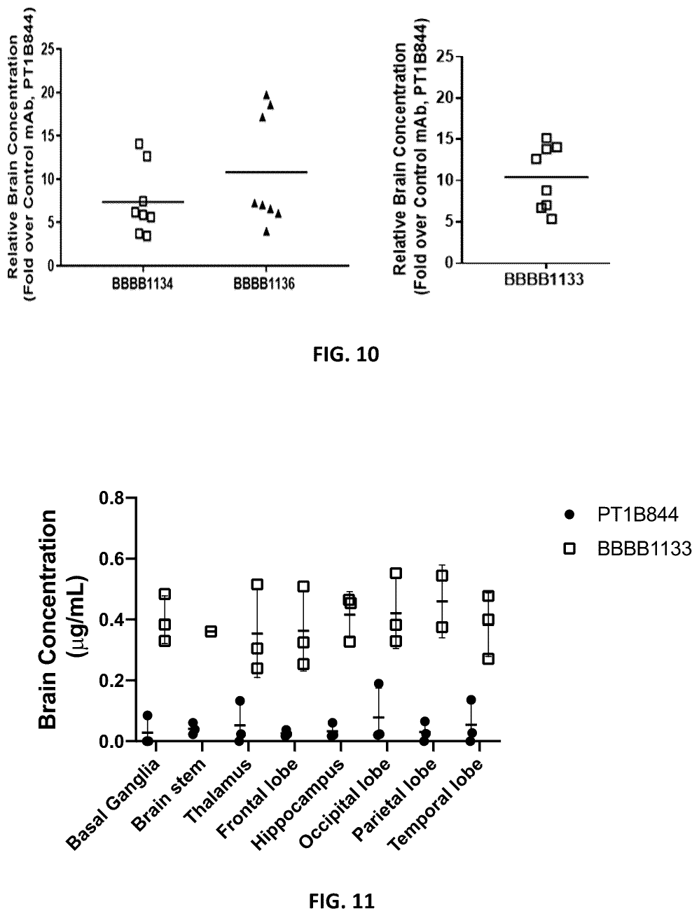

Brain concentration was determined for the mAbs across a variety of areas (FIG. 10). Brain concentration data were averaged across animals, and each symbol represents a region of the brain. A 7×, 11× and 11× greater brain concentration was observed for BBBB1134, BBBB1136 and BBBB1133 respectively, compared with the control mAb. All brain shuttle containing mAbs had increased brain exposure over the non-brain shuttle containing mAbs in every region of the brain (FIG. 11).

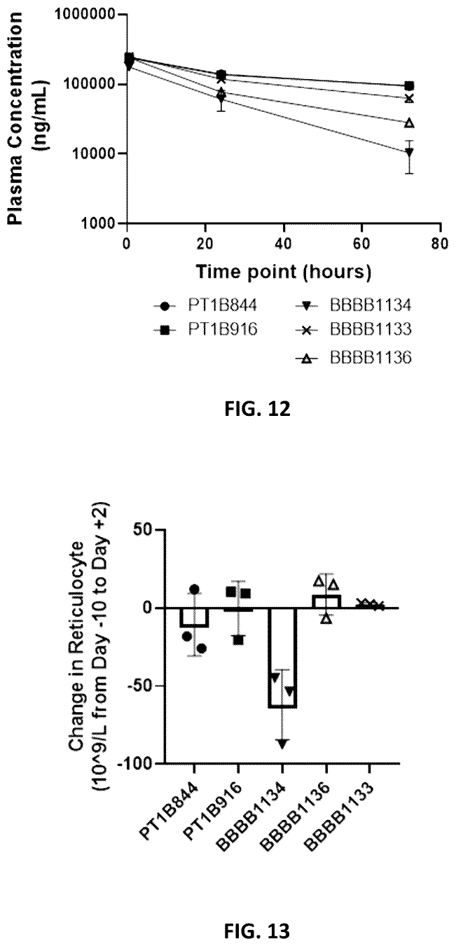

The concentration of mAb in plasma was also determined (FIG. 12). Evidence for TMDD was observed in the periphery, with the tripod mAbs having accelerated clearance over the control mAb (FIG. 18). The impact of binding to the neonatal Fc receptor (FcRn) was evaluated in this study with BBBB1134 and BBBB1136 being identical except in the Fc domain with BBBB1136 having the “YTE” mutation (Dall'Acqua, K, et al. 2006). The “YTE” mutation enhances binding to FcRn at acidic pH and has been demonstrated to increase the half-life of mAbs in multiple species, including humans (Robbie, C, et al. 2013). As would be anticipated, the addition of the “YTE” mutation resulted in an increased plasma concentration for BBBB1136 compared with BBBB1134. While FcRn is a critical receptor in maintaining IgG homeostasis and extending the serum half-life of IgG in humans (Roopenian and Akilesh 2007) it has also been implicated as a reverse transcytosis, or efflux, receptor from the brain (Cooper, C, et al. 2013). We were interested in understanding the interplay between these two functions for FcRn, as improving half-life by increasing the binding affinity for FcRn may have come at the expense of brain exposure with increased brain efflux. Interestingly, the 2-fold increase in plasma concentration was mirrored by a 2-fold increase in brain concentration suggesting that any potential increase efflux is negligible in this system.

BBBB1133 had a peripheral half-life most like the mAbs without the brain shuttle, PT1B844 and PT1B916.

### Reticulocyte Depletion in Cynomolgus Monkey

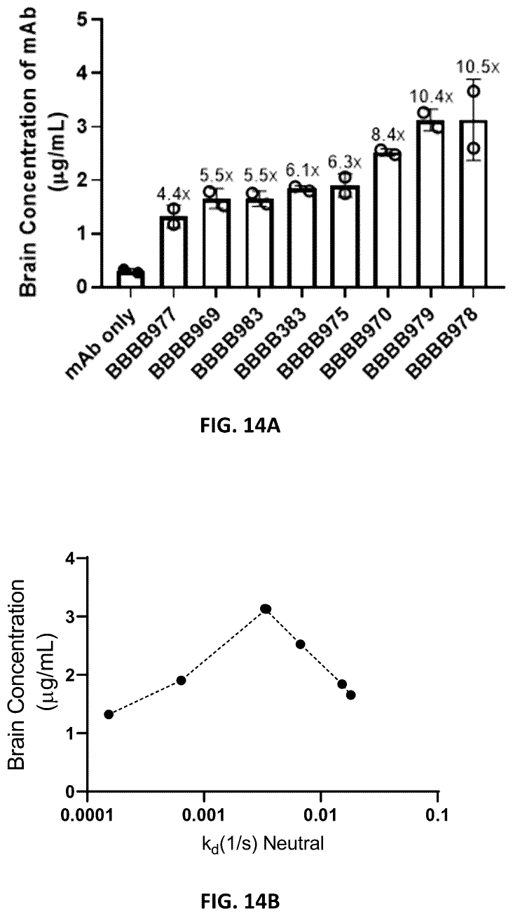

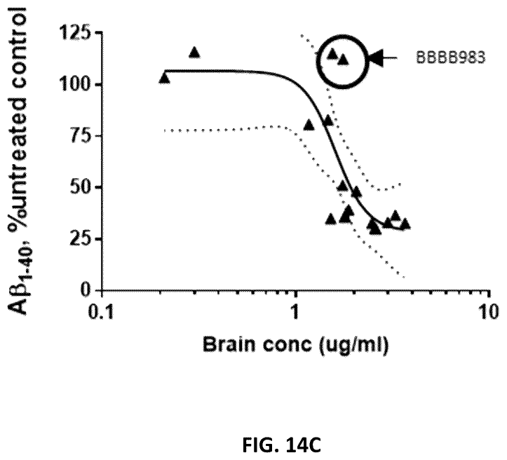

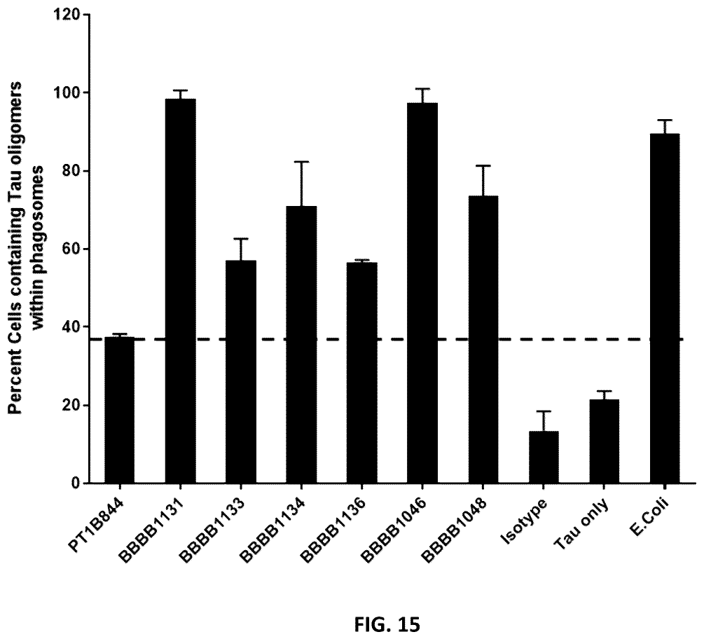

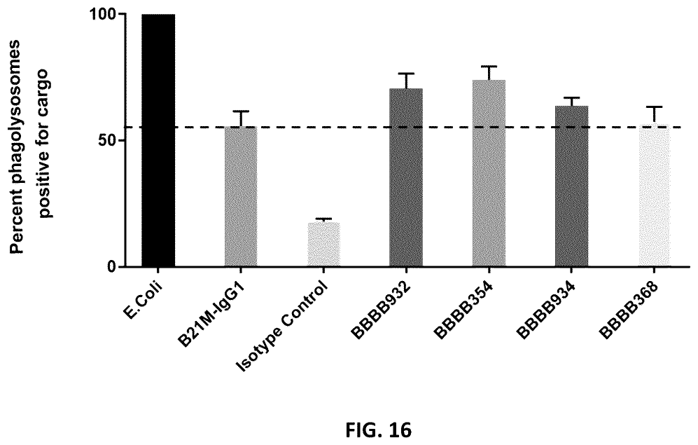

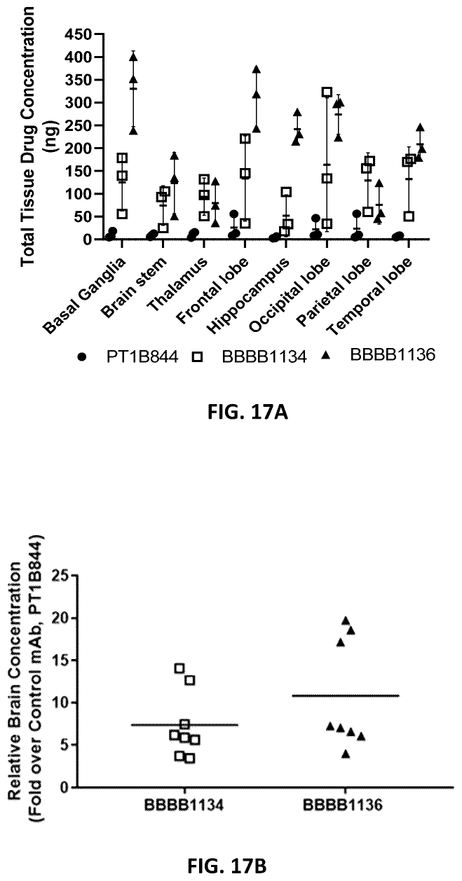

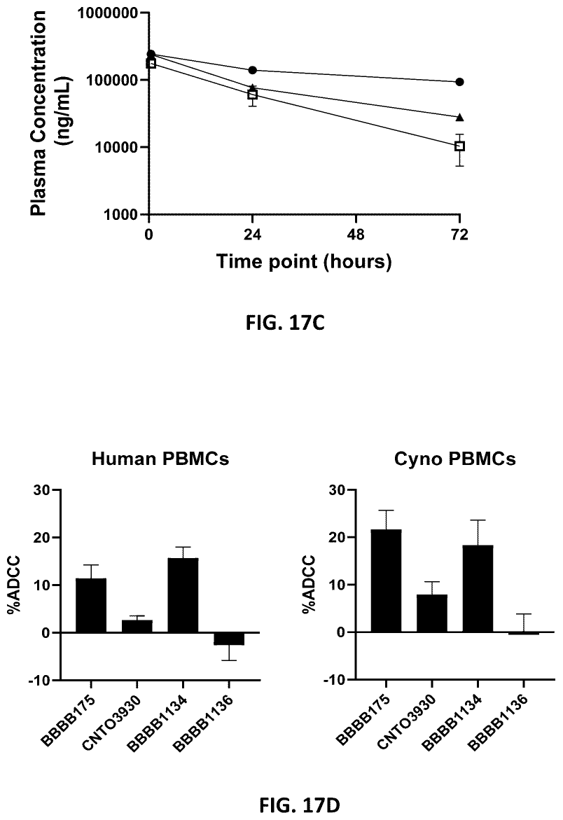

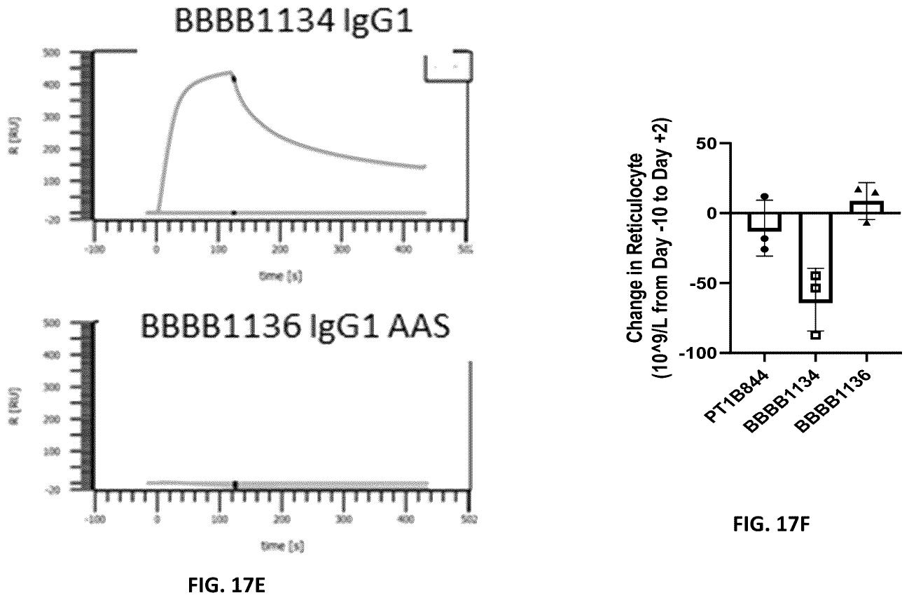

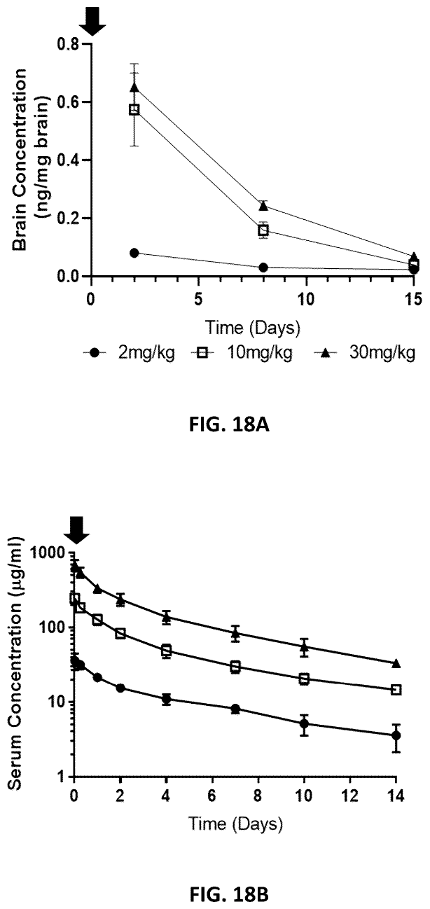

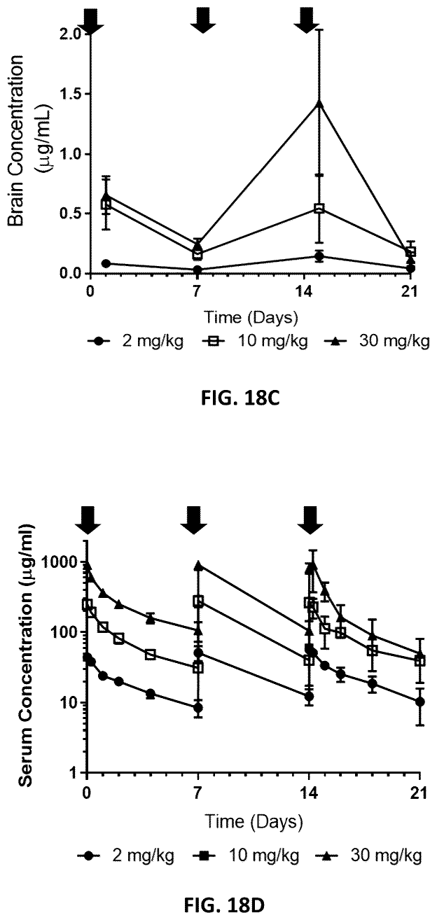

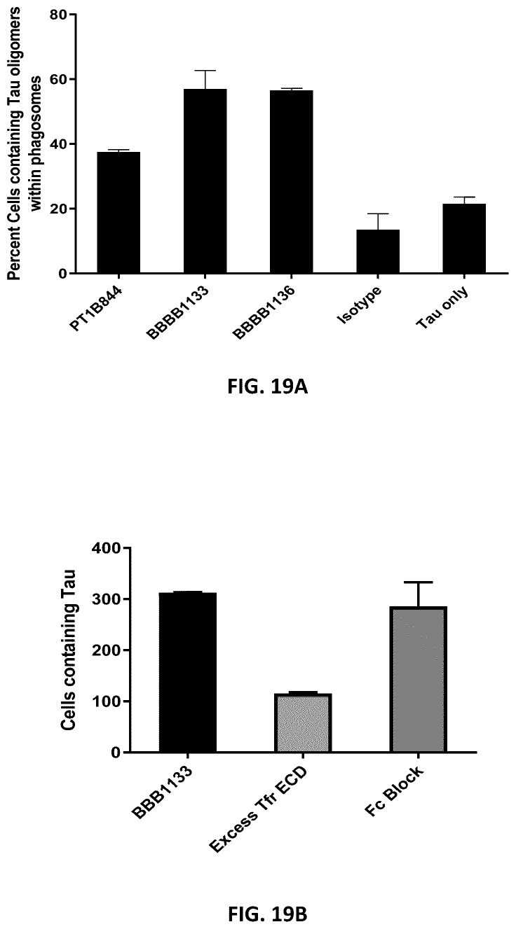

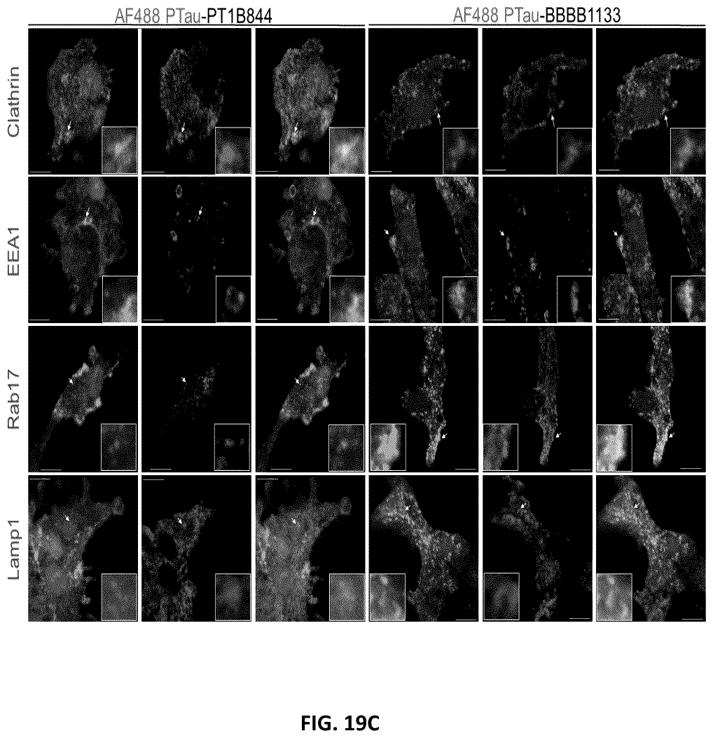

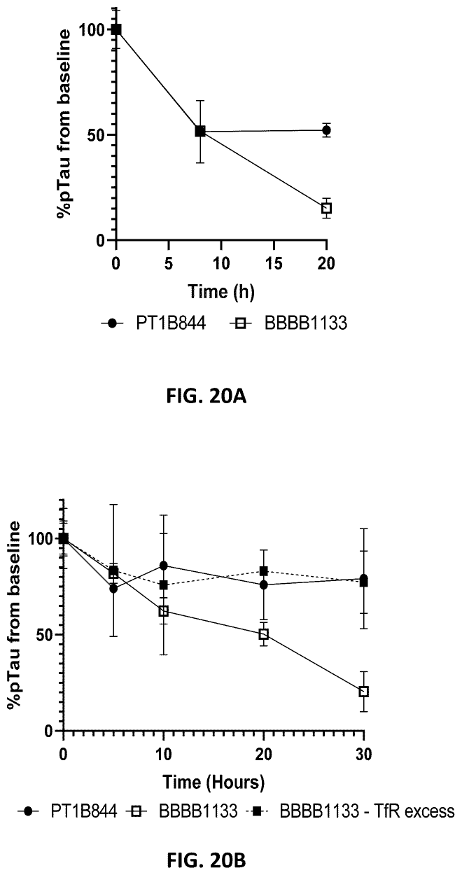

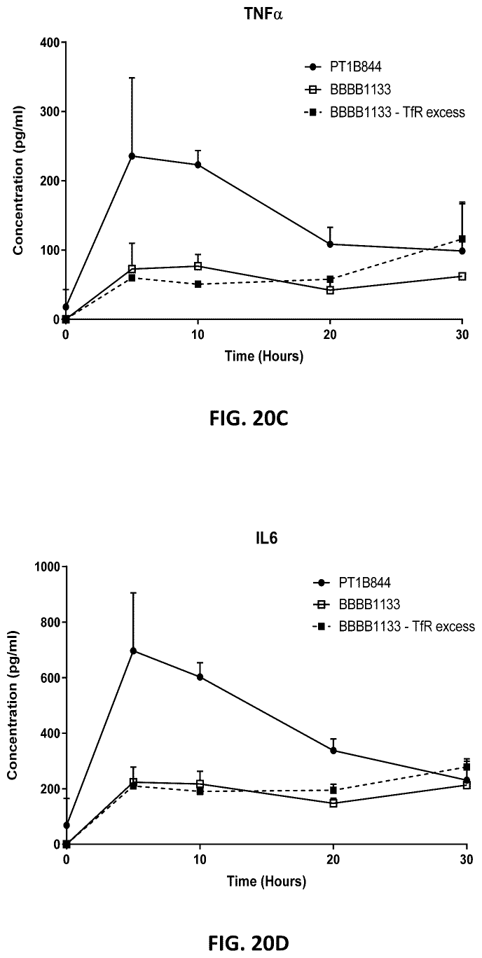

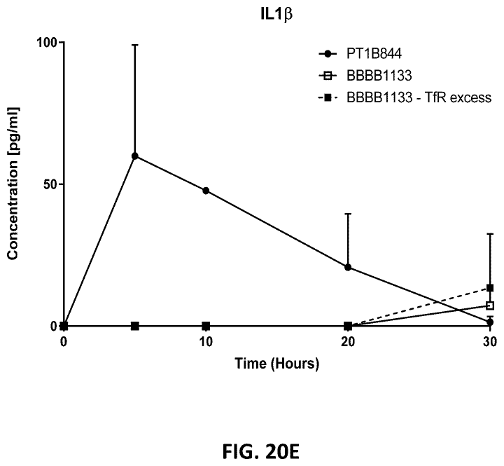

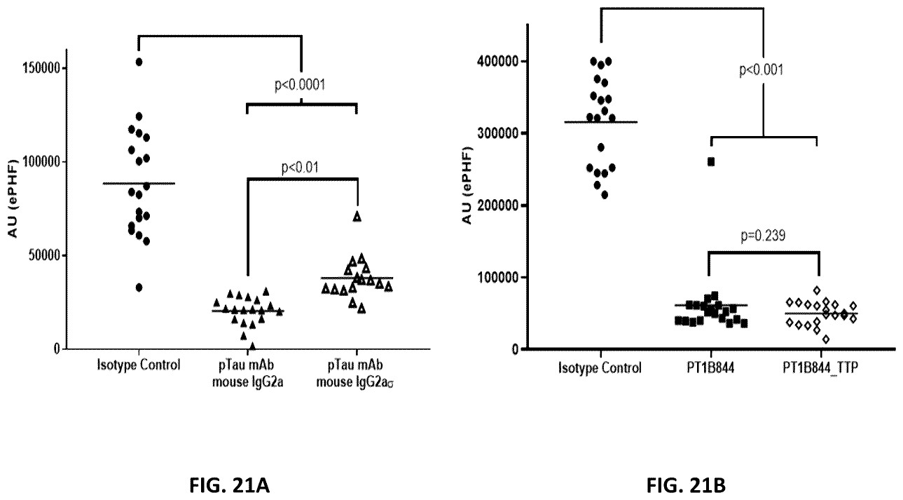

A known liability for TfR targeting to enhance brain exposure is reticulocyte depletion due to antibody-dependent cell-mediated cytotoxicity (ADCC) of reticulocytes in an Fc-dependent fashion (Science Translational Medicine 2013: Vol. 5, 183). mAbs were tested with WT IgG1 (BBBB1134) and the mutations “AAS” (BBBB1136 and BBBB1133) to reduce FcγR binding for reticulocyte depletion in the cyno PK study. As expected, rapid reticulocyte depletion was observed for the WT IgG1 tripod mAb, BBBB1134, but not observed for BBBB1136, BBBB1133 or the non-brain shuttle mAbs PT1B844 and PT1B916 (FIG. 13), confirming the impact of Fc function on TfR binding mAbs and reticulocyte depletion.

A third tripod mAb, BBBB1133, was selected for dose-response and repeat dosing cynomolgus monkey PK. Cynomolgus monkeys were dosed intravenously at 2, 10 and 30 mg/kg and brain PK determine 48 hours, 7- and 14-days later. Plasma PK was assessed over two weeks (FIGS. 18A and B). Linear brain PK was observed between 2 and 10 mg/kg and nonlinear brain PK between 10 and 30 mg/kg. The proposed mechanism of delivery is receptor-mediated, which will be saturable, and the data indicated that 30 mg/kg is a saturating dose in cynomolgus. Linear PK was observed in plasma and CSF with a half-life of approximately 6 days. Repeat dosing was also completed using the same dose ranges dosed weekly for three weeks (FIGS. 18C and D). Evidence for accumulation with repeated dosing of 30 mg/kg was observed and is aligned with the previous observation that 30 mg/kg is a saturating dose. Linear PK was observed once again the periphery with no evidence for PK tolerance with repeat dosing.

The reticulocyte data indicated that effector silent Fc mAbs are required for the safe dosing of this brain delivery platform. While avoiding reticulocyte depletion is an important characteristic for the safety of the therapeutic mAb, this requirement though would prevent using anti-TfR mediated brain delivery for any therapeutic mAb that requires effector function, like ADP, for the therapeutic mechanism of action. For example, one potential therapeutic mechanism of action relies on Fc-dependent microglia phagocytosis of Tau aggregates. By inhibiting the ability of the brain shuttle mAb to bind to FcγR to prevent reticulocyte depletion, the mAb would not be able to bind FcγR on microglia cells to promote phagocytosis of Tau aggregates.

To explore alternative pathways for ADP, we assessed the ability of the effector silent tripod mAbs, BBBB1133 and BBBB1136, to induce phagocytosis of Tau oligomers in human IPSC derived microglia cells. Both tripod mAbs induced greater phagocytosis of Tau oligomers than the control anti-Tau mAb, PT1B844, an IgG1 mAb (FIG. 19A). The ADP of Tau oligomers by BBBB1133 has been demonstrated to occur through TfR mediated internalization and can be blocked with the addition of an excess of soluble TfR extracellular domain. Addition of excess of soluble Fc does not impact ADP, confirming that non-classical ADP utilizes the TfR and not FcγRs (FIG. 19B). Similar intracellular trafficking of Tau was observed for BBBB1133 as the control mAb (PT1B844) through early endosomes (EEA1) to intermediate endosomes (Rab17) and finally the lysosome (LAMP1) (FIG. 19C).

To further validate non-classical ADP as a physiological relevant mechanism for Tau degradation by microglia, we assessed the ability of the tripod mAbs to induce phagocytosis of human postmortem Alzheimer's disease brain-derived tau fibrils (PHF-Tau). ADP of PHF-Tau was measured in both human monocyte derived macrophages and human IPSC derived microglia cells (FIG. 20). Both PT1B844 and BBBB1133 induced the phagocytosis of PHF-Tau at early timepoints. However, at the later timepoints BBBB1133 continues to induce ADP of PHF-Tau while PT1B844 mediated ADP stalls. This could be evidence for macrophage and microglial exhaustion as has been described for classical ADCP (Church, VanDerMeid et al. 2016) and a potential advantage of non-classical ADP mechanism utilized by BBBB1133. Similar to the previously described experiment using Tau oligomers, uptake of Tau is blocked with addition of an excess amount of soluble TfR demonstrating that this is a TfR-dependent mechanism. To probe another potential advantage of non-classical ADP over classical ADP, pro-inflammatory cytokines were measured in the PHF Tau phagocytosis experiment. As expected, classical ADP mediated by PT1B844 resulted in the secretion of proinflammatory cytokines while non-classical ADP mediated by BBBB1133 did not.

To assess the potential of the AAS IgG1 tripod mAbs to promote uptake of Tau aggregates in microglia cell, human microglia derived from Induced Pluripotent Stem Cells (iPSC) were plated onto 384 well Perkin Elmer Cell Carrier Ultra plates at a dilution of 7000 cells per well and maintained in advanced DMEM/F12 media with Glutamax+, Penicillin/Streptomycin, IL34 (100 ng/ml), and GMCSF (10 ng/ml). On the day of the assay, biotinylated phospho-tau oligomers [sequence: SCBiot-(dPEG4)GTPGSRSR(pT)PSLP(pT)PPTREPLL (SEQ ID NO: 315)-amide]were allowed to complex with streptavidin Alexa Fluor 488 (AF488) at 15-fold molar excess. Labelled phospho-tau oligomers were then allowed to bind test mAbs at approximately 2× molar excess at room temperature for 30 minutes. The mAb:tau oligomer complex was then delivered to microglia at 20 ul/well. At 2, 4, and 8 hours post incubation, cells were washed twice with phosphate buffered saline (PBS) and fixed in the presence of 4% paraformaldehyde for 15 minutes at room temperature. Following fixation, cells were once again washed twice in PBS and incubated overnight with LAMP1 primary antibody, a marker for lysosomes, at a concentration of 4 ug/ml in permeabilization buffer (0.1% saponin+1% Fish skin gelatin) at 4° C. Post incubation, cells were washed twice with PBS and stained with 1 ug/ml secondary antibody conjugated to Alexa Fluor 647 in permeabilization buffer for 1 hour at 4° C. Post incubation, cells were washed twice with PBS, counter-stained with Hoechst DNA stain at 1 ug/ml for 10 minutes at room temperature in PBS. The cells were then washed one final time in PBS, resuspended in 20 ul of PBS per well and imaged on the Opera Phenix confocal high content microscope. Acquired images were analyzed using Harmony and ImageJ analysis software. Approximately 500 cells per condition were scored for the presence of Tau oligomers within phagolysosomal structures, and labelled with LAMP1 antibody.

All brain shuttle mAbs promoted more efficient uptake into phagosomes than the non-brain shuttle mAb, PT1B844 (FIG. 15). Within the brain shuttle mAbs those with full effector function (BBBB1131, 1134 and 1046) were more efficient than those without effector function. These data demonstrate that eliminating binding to FcγR to reduce the risk of reticulocyte depletion should not impact therapeutic efficacy of the anti-Tau mAbs. In fact, TfR-mediated internalization and trafficking to the phagolysosome appears more efficient in microglia than did traditional FcγR mediated phagocytosis.

To explore if the observation could be repeated using other targets and cells, the uptake of RSV F-protein was assessed in human macrophages. Primary human macrophages were plated onto 384 well Perkin Elmer Cell Carrier Ultra plates at a dilution of approximately 6000 cells per well and cultured in X-VIVO 10 serum-free hematopoietic cell medium supplemented with 10% FBS, 50 mg/ml macrophage colony-stimulating factor (mCSF) CSF and 25 ng/ml interferon gamma (IFNγ). On the day of the assay, approximately 7-fold molar excess RSV-F protein (His-tagged F protein complexed with anti-His biotinylated antibody and streptavidin Alexa Fluor 488) was allowed to bind anti-RSV mAbs (1 ug/ml) at room temperature for 30 minutes. The mAb:F protein complex was then delivered to macrophages at 20 ul/well. Alexa Fluor 488 labelled E. Coli served as a positive control for phagocytosis. 3 hours post incubation, cells were washed twice with phosphate buffered saline (PBS) and fixed in the presence of 4% paraformaldehyde for 15 minutes at RT. Following fixation, cells were once again washed twice in PBS and incubated overnight with LAMP1 primary antibody, a marker for lysosomes, at a concentration of 4 ug/ml in permeabilization buffer (0.1% saponin+1% Fish skin gelatin) at 4° C. Post incubation, cells were washed twice with PBS and stained with 1 ug/ml secondary antibody conjugated to Alexa Fluor 647 in permeabilization buffer for 1 hour at 4° C. Post incubation, cells were washed twice with PBS, counter-stained with Hoechst DNA stain at 1 ug/ml for 10 minutes at room temperature in PBS. The cells were then washed one final time in PBS, resuspended in 20 ul of PBS per well and imaged on the Opera Phenix confocal high content microscope. Acquired images were analyzed using Harmony and ImageJ analysis software. Approximately 300 cells per condition were scored for the presence of F protein foci within phagolysosomal structures, labelled with LAMP1 antibody.

As was observed for Tau and microglia cells, all brain shuttle mAbs promoted more efficient uptake into phagosomes than the non-brain shuttle mAb, B21M-IgG1 (FIG. 16). However, a difference in uptake between IgG1 (BBBB932 and BBBB934) and IgG1 AAS (BBBB354 and BBBB368) brain shuttle mAbs was not observed. It remains to be determined if a difference between the B21M experiment and the Tau experiment (FIG. 15 and FIG. 16) is due to the target or the cells. Regardless, the data confirm the robustness of the mechanism where TfR-mediated internalization and trafficking to the phagolysosome appears at least as efficient as traditional FcγR mediated phagocytosis.

To the knowledge of the inventors, there has not been any publication describing exploiting this non-classical ADP mechanism for a therapeutic mAb. While not wishing to be bound by theories, it is believed that phagocytosis and endocytosis can both lead to degradation through the convergence of the phagolysosomal paths, such that regardless of the internalization trigger (FcγR mediated phagocytosis or TfR-mediated endocytosis), the internalized cargo is trafficked to and degraded by the phagolysosome.

Assessment of the PK/PD Relationship in the Retina of huTfR Mice

Selected anti-TfR brain shuttles were then fused to a prototypical anti-BACE ((3-secretase) mAb and binding affinity was reassessed using same method as described above. As shown in Table 5, the affinity of the anti-TfR brain shuttles was similar when fused to B21M mAb (anti-human respiratory syncytial virus) and anti-BACE antagonist mAb. Internalization was assessed for selected molecules (FIG. 4) and found unchanged from internalization observed when the anti-TfR brain shuttle was fused to B21M mAb.

Since none of the anti-TfR brain shuttles bound to mouse or rat TfR, in vivo rodent studies will be conducted in huTfR knock-in mice (C57BL/6-Tfrctm2618(TFRC)Arte mice (Taconic Artemis)) using the prototypical anti-BACE antagonist mAb (BBBB970, BBBB978, BBBB983). The anti-BACE antagonist mAb was selected as a model PD system for measuring inhibition of BACE1 (through the concentration of its product peptide, Aβ 1-40), a reflection of the amount of mAb that was trafficked to the brain.

The first in vivo study will assess the PK/PD relationship in the retina of huTfR mice. The knock-in (KI) mice will be dosed at 10 mg/kg i.v. with the BBBB970, BBBB978, BBBB983 and the control BBBB456. Eyes and plasma will be harvested at 4- and 24-hours following dosing. At the scheduled timepoints, mice will be anesthetized by inhalation of isoflurane. Mouse eyes from KI mice will be collected following whole-body perfusion with 5 mL of 0.9% saline solution. The collected eye sample (minus the optic nerve) will be snap frozen in liquid nitrogen, and stored at −70° C. until tissue homogenization or prepared for immunohistochemistry.

BACE activity measurements will be made by homogenizing mouse eyes in lysing matrix D tube (8 μl of 0.4% DEA/50 mM NaCl per mg of brain weight, Fast Prep-24 at 6/shakes/sec for 20 sec). Tubes will then be centrifuged at 4° C. for 5 min in an Eppendorf Centrifuge set to a maximum speed. Homogenate (supernatant) will then transferred to precooled tubes which were then centrifuged for 70 minutes at 13,000 rpm at 4° C. Supernatant will then be transferred to a tube containing 10% of 0.5 M Tris/HCL and frozen at −80° C. until assayed. Aβ 1-40 peptide standards and thawed processed eye homogenate are then pre-complexed at 1:1 with ruthenium (Meso Scale Discovery (MSD), R91AN-1) labeled anti-Aβ antibody. 50 ul of complex will be added to blocked plate containing capture antibody to Aβ 1-40. After overnight incubation at 2-8° C. with no shaking, plates will be washed and 2× read buffer (MSD, R92TC-1) added. Plate will be read using Meso Sector S 600 (MSD, ICOAA).

### Cytokine Secretion Analysis

After different treatments on human iPSC-derived microglia, the relative concentrations of secreted proteins in cell supernatants were measured using antibody-based 29-plex immunoassays (Luminex, R&D systems, Cat. #LXSAHM-29). The 29 secreted proteins were: BDNF, CCL3/MIP1α, CCL20/MIP3α, Grop/MIP2, CXCL10/IP10/CRG2, GCSF, IFNα, IL1α, IL2, IL6, IL10, IL17/IL17α, MCSF, RAGE/AGER, TNFα, CCL2/JE/MCP1, CCL4/MIP1β, CXCL9/MIG, FGFb/FGF2, GMCSF, IFNγ, IL13, IL4, IL8/CXCL8, IL12p70, IL23, MMP9, Resistin.

### PHF Tau

Postmortem tissue from the cortex obtained from 5 histologically confirmed AD patient (Braak stage V-VI) was used to generate a pool of partially purified PHF by a modified method of (Mercken et al., Acta Neuropathologica (1992) 84: 265-272; Greenberg, et al. J. biol. Chem. (1992) 267: 564-569). Typically, 5 g of parietal or frontal cortex was homogenized in 10 volumes of cold buffer H (10 mM Tris, 800 mM NaCl, 1 mM EGTA and 10% sucrose/pH 7.4) using a glass/Teflon Potter tissue homogenizer (IKA Works, Inc; Staufen, Germany) at 1000 rpm. The homogenized material was centrifuged at 27000×g for 20 min at 4° C. The pellet was discarded, and the supernatant was adjusted to a final concentration of 1% (w/v) N-lauroylsarcosine and incubated for 2 h at 37° C. Subsequently, the supernatant was centrifuged at 184000×g for 90 min at 20° C. The pellet was carefully washed in PBS and resuspended in 750 uL PBS, aliquoted and frozen at −80° C. The quality of the PHF-tau preparations was evaluated by the use of AT8/AT8 phospho-aggregate selective MSD ELISA. Tau content was determined by western blotting using hTau10 (Janssen R&D) with recombinant 2N4R tau as calibrant.

### Study Re the Ability of the TfR TTP mAb to Potentiate ADP In Vivo

The ability of the TfR TTP mAb to potentiate ADP in vivo was studied in a mouse model of Tau seeding. The mouse model employed transgenic Tau-P301L mice, expressing the longest human tau isoform with the P301L mutation (tau-4R/2N-P301L) (Terwel, et al. (2005) J Biol Chem; 280(5): 3963-73). Due to the lack of mouse TfR cross-reactivity of the TTPs, a mouse surrogate TTP was developed to have similar binding properties to the lead human TfR TTP and used in this study. The model of Tau seeding involves stereotactic hippocampal injections of PHF-Tau, which induces a dose-dependent increase in tau aggregation (Vandermeeren, et al., J Alzheimers Dis. (2018); 65(1): 265-281). Following co-injection of mAbs, neutralization of Tau seeding by different anti-tau mAbs has demonstrated that the model is partially dependent upon Fc-mediated ADP of Tau (FIG. 21A). While both anti-Tau mAbs neutralized Tau seeding compared with the isotype control, a statistically significant difference is observed between the mAb with effector function (mouse IgG2a) and the mAb without effector function (mouse IgG2aσ (Vafa, et al., Methods. 2014 Jan. 1; 65(1): 114-26)), demonstrating the partial dependence of the model on mAb effector function.

A similar study was completed comparing the anti-Tau mAb, PT1B844 with a mouse IgG2a Fc, to the PT1B844 TTP mAb with a human IgG1 AAS Fc. Co-injection of mAbs was utilized to normalize for any differences in PK properties between the mAb and the TTP mAb. Both anti-Tau mAbs neutralized Tau seeding compared with the isotype control. TTP mAb displayed at least the same compared to the mAb with full Fc effector function, suggesting that non-classical ADP mechanism is functional in vivo (FIG. 21B).

### Stereotactic Injection of PHF in P301L Mice

PHF tau seeding studies, including the currently described study, are performed in compliance with protocols approved by the local ethical committee (628-Tau Spread, Janssen Pharmaceutica) and national institutions adhering to AAALAC guidelines. Mice expressing the longest human tau isoform with the P301L mutation (tau-4R/2N-P301L) (Terwel et al., 2005; Peeraer et al., 2015) were single housed in an enriched environment, individually ventilated cages and under 12/12 h light/dark cycles (light on at 6:00 AM). At the age of 90+/−7 days, mice were randomized over treatment groups and gender and received a unilateral injection in the right hippocampus (CA1) of AD-derived PHFs (in the presence of anti-IgG2a (n=19); anti phospho Tau mouse IgG2a (n=20) or anti-phospho Tau-TTE (n=20).

Tau.P301L mice were deeply anaesthetized with isoflurane (5% in 36% oxygen) and fixed in a stereotactic frame (Stoelting-Neurostar combination). During the further procedure a 2% isoflurane level was maintained. A 30G syringe (Hamilton) was used for injecting 3 L in the right hemisphere at a speed of 0.25 l/min at the selected coordinates: anteroposterior −2.0, mediolateral+1.6 from bregma, dorsoventral 1.4 mm from dura. Body weight was monitored before and weekly after injection, and no differences were observed between treatment and control groups for all injection experiments (not shown).

Two months after injection, mice were sacrificed by decapitation and brain tissue from the ipsilateral hemisphere was snap frozen. Before extraction, tissue was weighed and homogenized in 600 μL of buffer H per 100 mg tissue (10 mM Tris, 800 mM NaCl, 1 mM EGTA and 10% sucrose/pH 7.4). The homogenate was centrifuged at 27 000×g for 20 min and supernatant was frozen at −80° C.

### Biochemical Analysis MesoScale Discovery (MSD)

Coating antibody (AT8) was diluted in PBS (1 μg/mL) and aliquoted into MSD plates (30 μL per well) (L15XA, MSD, Rockville, MD, USA), which were incubated overnight at 4° C. After washing with 5×200 μL of PBS/0.5% Tween-20, the plates were blocked with 0.1% casein in PBS and washed again with 5×200 μl of PBS/0.5% Tween-20. After adding samples and standards (both diluted in 0.1% casein in PBS), the plates were incubated overnight at 4° C. Subsequently, plates were washed with 5×200 μL of PBS/0.5% Tween-20, and SULFO-TAG™ conjugated detection antibodies (AT8) in 0.1% casein in PBS were added and incubated for 2 h at room temperature while shaking at 600 rpm. After a final wash (5×200 μL of PBS/0.5% Tween-20), 150 μL of 2×buffer T (MSD) was added, and plates were read with an MSD imager. Raw signals were normalized against a standard curve consisting of 16 dilutions of a sarcosyl-insoluble prep from postmortem AD brain (PHF) and were expressed as arbitrary units (AU) PHF. Statistical analysis (ANOVA with Bonferroni correction for multiple testing) was performed with the GraphPad prism software. P-values<0.05 were considered as significantly different.

### Discussion

To achieve an optimized brain delivery platform based on receptor mediated transcytosis, mAbs were generated that bind specifically to the human transferrin receptor (huTfR) with a range of affinities in a pH-dependent manner. The relationship between TfR binding affinity and transcytosis efficiency has been covered extensively in numerous publications with a focus on the equilibrium dissociation constant, K_{D}. While K_{D} is an important measure, it has been surprisingly demonstrated in the invention the criticality of the binding kinetics, kₐ and k_{d}, for transcytosis. Inventors discovered that both on- and off-rates need to be optimized for efficient transcytosis and pharmacodynamic activity of the therapeutic mAb delivered. Based on the results, optimal transcytosis occurs when, for example, the kₐ≥10⁵ M⁻¹ s⁻¹ and neutral k_{d}=2×10⁻³ sec⁻¹. While not wishing to be bound by theories, it is hypothesized the interplay between on-rate and off-rate is critical to ensuring efficient transcellular transport through the diverse intracellular vesicles responsible for protein trafficking in polarized cells

It has been demonstrated that dosing tripod mAbs in cynomolgus monkeys results in 6-12× enhancement in brain concentration over a control mAb. Increasing acidic FcRn binding resulted in reduced peripheral clearance and enhanced brain concentration. Under normal physiological conditions, FcRn-mediated efflux of antibodies from the brain is likely crucial in maintaining brain homeostasis by avoiding unwanted inflammation and immune responses in the brain (Schlachetzki, Zhu et al. 2002, Roopenian and Akilesh 2007). While the preponderance of evidence suggests a strong role for FcRn-mediated efflux of antibodies, there does remain some debate about this clearance mechanism (Garg and Balthasar 2009, Abuqayyas and Balthasar 2013). Inventors discovered that increasing the binding affinity for FcRn has a positive impact on both peripheral and brain concentration, suggesting that any enhanced efflux is insignificant in this system. Dose response experiments in cynomolgus monkeys using a tripod mAb demonstrated the saturability of the mechanism of transport, which occurs at 30 mg/kg in this species. Extensive repeat dosing, dose response characterization was also completed in cynomolgus and will aid greatly in predicting human doses and the utility of this platform for specific therapeutic applications.

Reticulocyte depletion is a known safety liability for TfR binding antibodies. It was observed by the inventors that indeed acute and nearly complete reticulocyte depletion can be observed with an effector function competent mAb. Numerous approaches have been described to avoid this depletion, including reducing effector function {Couch, 2013 #589} and through molecular architecture {Weber, 2018 #590}. While inventors utilized a very similar architecture to one that has been described as sterically capable of attenuating peripheral effector function, they observed robust reticulocyte depletion with the effector function competent mAb.

The clear disadvantage to Fc mutagenesis is the elimination of effector function from the therapeutic mAb. For many therapeutic targets in the brain, like beta-amyloid and Tau, ADP is believed critical to efficacy. Previous work has demonstrated that recycling receptors, including TfR, can be excluded from sorting tubules and diverted to lysosomes by multivalent cargo binding (Marsh, 1995, J Cell Biol (1995) 129 (6): 1509-1522; Weflen, 2013 Mol Biol Cell. 2013 Aug. 1; 24(15): 2398-24050. Inventors demonstrated that this endogenous diversion of multivalent cargo can be used as an alternative, non-classical, non-FcγR mechanism of ADP. Tau internalized through non-classical and classical ADP are trafficked similarly in microglia, with Tau aggregates trafficking through the endolysosomal system to the lysosome for degradation. The described non-classical ADP can be exploited for a variety of therapeutic applications where ADP is necessary for efficacy but classical ADP harmful for safety.

The data indicate that non-classical ADP is more efficient than classical ADP, potentially due to inherent differences in the binding and internalization between FcγRs and TfR. FcγR mediated internalization requires receptor clustering by the mAb, while TfR is rapidly internalizing and recycling independent of mAb binding. A second potential explanation is macrophage and microglial exhaustion (Zent, 2017 FEBS J. 2017 April; 284(7):1021-1039). Macrophage exhaustion appears to be dependent upon the length of time the macrophage is exposed to the target (Church, VanDerMeid et al. 2016, Clin Exp Immunol. 2016 January; 183(1):90-101) (Mukundan, 2009, Nat Med. 2009 November; 15(11):1266-72), which is aligned with our observation of that classical ADP stalls with time. Observations for macrophage exhaustion have been made in vitro and in patients, indicating that this exhaustion phenotype may impact therapeutic efficacy of mAbs with effector function. The non-classical ADP offers an efficacy advantage, avoiding this exhaustion phenotype by mediating ADP without activating microglia by binding FcγRs.

Another advantage of non-classical ADP is that by avoiding microglial activation ADP occurs without stimulating the production pro-inflammatory cytokines. There remains debate on the safety of using effector-function competent mAbs in treating diseases in the brain, particularly around increasing neuroinflammation in patients who already suffer from chronic neuroinflammation (reviewed in {Heneka, 2015 #591}). In addition, there is increasing attention to the role that inflammation plays in the pathogenesis of neurodegenerative disease with the implications of increasing inflammation, as well as, the ability to engage or further activate potentially already exhausted microglia under debate. For example, the toxic impact of classic ADP on neurons was been demonstrated and hypothesized that effector function competent mAbs may pose safety risks {Lee, 2016 #592}. The non-classical ADP mechanism described here avoids the potential neuroinflammation liabilities by potentiating efficient clearance of Tau without needing to activate microglia or stimulate release of proinflammatory cytokines. In conclusion, a robust brain delivery platform has been characterized for pharmacokinetics, pharmacodynamics and safety establishing the robust preclinical characterization needed to advance to clinical trials.

When formatted as scFv brain shuttles and fused to a prototypical anti-BACE (beta-secretase) antagonist mAb, a 4-10× improvement in brain concentration was observed over the anti-BACE mAb alone following i.v. dosing of transgenic mice expressing huTfR. A strong PK:PD relationship was also noted, with a dose dependent decrease in beta-amyloid detected. The best performing brain shuttles enhanced brain delivery more than competitor molecules, achieving best-in-class delivery through optimized binding interactions between the brain shuttle and huTfR.

The optimized brain shuttles were then fused to PT1B844, a Tau binding mAb. Brain shuttles fused PT1B844 demonstrated 6 to 16-fold improvement in brain concentration when dosed i.v. in cynomolgus monkey. Similar to the mouse data, the enhancement in brain concentration exceeded the best brain shuttles reported in literature. In addition to superior brain PK, the brain shuttles are engineered to reduce Fc-mediated effector function and do not induce rapid reticulocyte depletion in cynomolgus as has been reported by competitors. Importantly, the loss of Fc-function doesn't impact the effectiveness of the therapeutic Tau mAb, as the brain shuttle is more efficient at mediating microglial uptake of Tau than PT1B844 alone.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### CITATIONS

Abuqayyas, L. and J. P. Balthasar (2013). “Investigation of the role of FcgammaR and FcRn in mAb distribution to the brain.” Mol Pharm 10(5): 1505-1513.
Church, A. K., K. R. VanDerMeid, N. A. Baig, A. M. Baran, T. E. Witzig, G. S. Nowakowski and C. S. Zent (2016). “Anti-CD20 monoclonal antibody-dependent phagocytosis of chronic lymphocytic leukaemia cells by autologous macrophages.” Clin Exp Immunol 183(1): 90-101.
Cooper, P. R., G. J. Ciambrone, C. M. Kliwinski, E. Maze, L. Johnson, Q. Li, Y. Feng and P. J. Hornby (2013). “Efflux of monoclonal antibodies from rat brain by neonatal Fc receptor, FcRn.” Brain Res 1534: 13-21.
Dall'Acqua, W. F., P. A. Kiener and H. Wu (2006). “Properties of human IgG1s engineered for enhanced binding to the neonatal Fc receptor (FcRn).” J Biol Chem 281(33): 23514-23524.
Garg, A. and J. P. Balthasar (2009). “Investigation of the influence of FcRn on the distribution of IgG to the brain.” AAPS J 11(3): 553-557.
Goulatis, L. I. and E. V. Shusta (2017). “Protein engineering approaches for regulating blood-brain barrier transcytosis.” Curr Opin Struct Biol 45: 109-115.
Johnsen, K. B., A. Burkhart, F. Melander, P. J. Kempen, J. B. Vejlebo, P. Siupka, M. S. Nielsen, T. L. Andresen and T. Moos (2017). “Targeting transferrin receptors at the blood-brain barrier improves the uptake of immunoliposomes and subsequent cargo transport into the brain parenchyma.” Sci Rep 7(1): 10396.
Robbie, G. J., R. Criste, W. F. Dall'acqua, K. Jensen, N. K. Patel, G. A. Losonsky and M. P. Griffin (2013). “A novel investigational Fc-modified humanized monoclonal antibody, motavizumab-YTE, has an extended half-life in healthy adults.” Antimicrob Agents Chemother 57(12): 6147-6153.
Roopenian, D. C. and S. Akilesh (2007). “FcRn: the neonatal Fc receptor comes of age.” Nat Rev Immunol 7(9): 715-725.
Schlachetzki, F., C. Zhu and W. M. Pardridge (2002). “Expression of the neonatal Fc receptor (FcRn) at the blood-brain barrier.” J Neurochem 81(1): 203-206.
Vandermeeren, M., M. Borgers, K. Van Kolen, C. Theunis, B. Vasconcelos, A. Bottelbergs, C. Wintmolders, G. Daneels, R. Willems, K. Dockx, L. Delbroek, A. Marreiro, L. Ver Donck, C. Sousa, R. Nanjunda, E. Lacy, T. Van De Casteele, D. Van Dam, P. P. De Deyn, J. A. Kemp, T. J. Malia and M. H. Mercken (2018). “Anti-Tau Monoclonal Antibodies Derived from Soluble and Filamentous Tau Show Diverse Functional Properties in vitro and in vivo.” J Alzheimers Dis 65(1): 265-281.
Yu, Y. J., J. K. Atwal, Y. Zhang, R. K. Tong, K. R. Wildsmith, C. Tan, N. Bien-Ly, M. Hersom, J. A. Maloney, W. J. Meilandt, D. Bumbaca, K. Gadkar, K. Hoyte, W. Luk, Y. Lu, J. A. Ernst, K. Scearce-Levie, J. A. Couch, M. S. Dennis and R. J. Watts (2014). “Therapeutic bispecific antibodies cross the blood-brain barrier in nonhuman primates.” Sci Transl Med 6(261): 261ra154.
Yu, Y. J., Y. Zhang, M. Kenrick, K. Hoyte, W. Luk, Y. Lu, J. Atwal, J. M. Elliott, S. Prabhu, R. J. Watts and M. S. Dennis (2011). “Boosting brain uptake of a therapeutic antibody by reducing its affinity for a transcytosis target.” Sci Transl Med 3(84): 84ra44.
Zent, C. S. and M. R. Elliott (2017). “Maxed out macs: physiologic cell clearance as a function of macrophage phagocytic capacity.” FEBS J 284(7): 1021-1039.

What is claimed is:

1. A conjugate comprising an anti-transferrin receptor (TfR) antibody or antigen-binding fragment thereof coupled to:
(i) a therapeutic or diagnostic agent; or
(ii) a second antibody or an antigen-binding fragment thereof that binds to a brain target;
wherein the anti-TfR antibody or antigen-binding fragment thereof comprises a heavy chain variable region comprising heavy chain complementarity determining regions (HCDRs) HCDR1, HCDR2, and HCDR3 and a light chain variable region comprising light chain complementarity determining regions (LCDRs) LCDR1, LCDR2, and LCDR3, wherein the HCDR1, HCDR2, HCDR3, LCDR1, LCDR2, and LCDR3 have the amino acid sequences of SEQ ID NOs: 292, 293, 294, 295, 296, and 297, respectively.

2. The conjugate of claim 1, wherein the anti-TfR antibody or antigen-binding fragment thereof is covalently linked to the second antibody or antigen-binding fragment thereof.

3. The conjugate of claim 1, wherein the anti-TfR antigen-binding fragment is a single-chain variable fragment (scFv) comprising an amino acid sequence having at least 90% sequence identity to the amino acid sequence of SEQ ID NO: 291 or 162.

4. The conjugate of claim 1, wherein the anti-TfR antigen-binding fragment is an scFv, and wherein the heavy chain variable region is covalently linked to the light chain variable region via a linker having the amino acid sequence of SEQ ID NO: 314.

5. The conjugate of claim 1, wherein the conjugate comprises a second antibody or an antigen-binding fragment thereof and is covalently linked to the carboxy terminus of only one of the two heavy chains of the second antibody or antigen-binding fragment thereof via a linker.

6. The conjugate of claim 5, wherein each of the two heavy chains of the second antibody or antigen-binding fragment thereof comprises:
(i) one or more heterodimeric mutations;
(ii) one or more mutations in the Fc domain that enhance binding of the fusion to the neonatal Fc receptor (FcRn); and/or
(iii) one or more mutations in the Fc domain that reduce or eliminate the effector function.

7. The conjugate of claim 6, wherein:
(i) the heterodimeric mutations comprise a modified heterodimeric CH3 domain or one or more knob and hole mutations, wherein the modified heterodimeric CH3 domain of the first heavy chain comprises amino acid modifications at positions T350, L351, F405, and Y407, and the modified heterodimeric CH3 domain of the second heavy chain comprises amino acid modifications at positions T350, T366, K392 and T394, wherein the amino acid modification at position T350 is T350V, T350I, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V or T366M, the amino acid modification at position K392 is K392F, K392L or K392M, and the amino acid modification at position T394 is T394W;
(ii) the one or more mutations in the Fc domain enhance binding of the conjugate to the neonatal Fc receptor (FcRn) at an acidic pH; and/or
(iii) the Fc domain comprises one or more amino acid modifications at positions L234, L235, D270, N297, E318, K320, K322, P331, and/or P329, and wherein the numbering of amino acid residues is according to the EU index as set forth in Kabat.

8. The conjugate of claim 7, wherein the modified heterodimeric CH3 domain of the first heavy chain comprises the mutations T350V, L351Y, F405A and Y407V; the modified heterodimeric CH3 domain of the second heavy chain comprises the mutations T350V, T366L, K392L and T394W; and the Fc has the M252Y/S254T/T256E (YTE) mutations and/or one, two or three mutations of L234A, L235A and P331S.

9. The conjugate of claim 1, wherein the anti-TfR antibody or antigen-binding fragment thereof is covalently linked to a second antibody or antigen-binding fragment thereof, wherein the anti-TfR antibody or antigen-binding fragment thereof is covalently linked to the carboxy terminus of only one of the two heavy chains of the second antibody or antigen-binding fragment thereof via a linker, wherein the anti-TfR antibody or antigen-binding fragment thereof comprises
an scFv comprising an amino acid sequence having at least 90% sequence identity to the amino acid sequence of SEQ ID NO: 291 or 162.

10. The conjugate of claim 1, wherein the brain target is selected from the group consisting of beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

11. A conjugate comprising an TfR antibody or antigen-binding fragment thereof coupled to:
(i) a therapeutic or diagnostic agent; or
(ii) a second antibody or an antigen-binding fragment thereof that binds to a brain target;
wherein the anti-TfR antibody or antigen-binding fragment thereof comprises:
(1) a heavy chain variable region comprising HCDRs HCDR1, HCDR2, and HCDR3 and a light chain variable region comprising LCDRs LCDR1, LCDR2, and LCDR3, wherein the HCDR1, HCDR2, HCDR3, LCDR1, LCDR2, and LCDR3 have the amino acid sequences of:
(i) SEQ ID NOs: 29, 30, 31, 32, 33 and 34, respectively;
(ii) SEQ ID NOs: 57, 58, 59, 60, 61 and 62, respectively;
(iii) SEQ ID NOs: 85, 86, 87, 88, 89 and 90, respectively;
(iv) SEQ ID NOs: 110, 111, 112, 113, 114 and 115, respectively;
(v) SEQ ID NOs: 135, 136, 137, 138, 139 and 140, respectively;
(vi) SEQ ID NOs: 191, 192, 193, 194, 195 and 196, respectively;
(vii) SEQ ID NOs: 244, 245, 246, 247, 248 and 249, respectively;
(viii) SEQ ID NOs: 263, 264, 265, 266, 267 and 268, respectively;
(ix) SEQ ID NOs: 345, 346, 347, and 348, WAS, and SEQ ID NO: 350, respectively;
(x) SEQ ID NOs: 355, 356, 357, and 358, TLS, and SEQ ID NO: 360, respectively;
(xi) SEQ ID NOs: 365, 366, 367, and 368, TVS, and SEQ ID NO: 370, respectively;
(xii) SEQ ID NOs: 375, 376, 377, and 378, GAS, and SEQ ID NO: 380, respectively;
(xiii) SEQ ID NOs: 385, 386, 387, and 388, KVS, and SEQ ID NO: 390, respectively;
(xiv) SEQ ID NOs: 395, 396, 377, and 398, SAS, and SEQ ID NO: 400, respectively;
(xv) SEQ ID NOs: 405, 406, 407, and 408, AAS, and SEQ ID NO: 410, respectively;
(xvi) SEQ ID NOs: 415, 416, 417, and 418, QDN, and SEQ ID NO: 420, respectively;
(xvii) SEQ ID NOs: 425, 426, 427, and 428, AAS, and SEQ ID NO: 430, respectively;
(xviii) SEQ ID NOs: 435, 436, 437, and 438, ASS, and SEQ ID NO: 440, respectively;
(xix) SEQ ID NOs: 445, 446, 447, and 448, AAS, and SEQ ID NO: 450, respectively;
(xx) SEQ ID NOs: 455, 456, 457, and 458, DNN, and SEQ ID NO: 460, respectively;
(xxi) SEQ ID NOs: 465, 466, 467, and 468, GAS, and SEQ ID NO: 470, respectively;
(xxii) SEQ ID NOs: 475, 476, 477, and 478, QDR, and SEQ ID NO: 480, respectively;
(xxiii) SEQ ID NOs: 485, 486, 487, and 488, QDT, and SEQ ID NO: 490, respectively;
(xxiv) SEQ ID NOs: 495, 496, 497, and 498, DDS, and SEQ ID NO: 500, respectively;
(xxv) SEQ ID NOs: 505, 506, 507, and 508, QDR, and SEQ ID NO: 510, respectively;
(xxvi) SEQ ID NOs: 515, 516, 517, and 518, QDN, and SEQ ID NO: 520, respectively;
(xxvii) SEQ ID NOs: 525, 526, 527, and 528, EVS, and SEQ ID NO: 530, respectively;
(xxviii) SEQ ID NOs: 535, 536, 537, and 538, QDT, and SEQ ID NO: 540, respectively; or
(xxix) SEQ ID NOs: 545, 546, 547, and 548, QDR and SEQ ID NO: 550, respectively; or
(2) a VHH comprising HCDRs HCDR1, HCDR2 and HCDR3 having the amino acid sequences of:
(i) SEQ ID NOs: 7, 8 and 9, respectively;
(ii) SEQ ID NOs: 317, 318 and 319, respectively;
(iii) SEQ ID NOs: 324, 325 and 326, respectively;
(iv) SEQ ID NOs: 331, 332 and 333, respectively; or
(v) SEQ ID NOs: 338, 339 and 340, respectively.

12. The conjugate of claim 11, wherein:
(1) the anti-TfR antigen-binding fragment is an scFv comprising an amino acid sequence having at least 90% sequence identity to the amino acid sequence of SEQ ID NO: 28, 56, 84,109, 134, 190, 218, 243, 262, 344, 354, 364, 374, 384, 394, 404, 414, 424, 434, 444, 454, 464, 474, 484, 494, 504, 514, 524, 534, or 544; or
(2) the anti-TfR antigen-binding fragment is a VHH fragment comprising an amino acid sequence having at least 90% sequence identity to SEQ ID NO: 6, 316, 323, 330, or 337.

13. The conjugate of claim 11, wherein the anti-TfR antibody or antigen-binding fragment thereof is covalently linked to a second antibody or antigen-binding fragment thereof, wherein the anti-TfR antibody or antigen-binding fragment thereof is covalently linked to the carboxy terminus of only one of the two heavy chains of the second antibody or antigen-binding fragment thereof via a linker, wherein the anti-TfR antibody or antigen-binding fragment thereof comprises:
(1) an scFv comprising an amino acid sequence having at least 90% sequence identity to the amino acid sequence of SEQ ID NO: 28, 56, 84, 109, 134, 190, 218, 243, 262, 344, 354, 364, 374, 384, 394, 404, 414, 424, 434, 444, 454, 464,474, 484, 494, 504, 514, 524, 534 or 544; or
(2) a VHH fragment comprising an amino acid sequence having at least 90% sequence identity to SEQ ID NO: 6, 316, 323, 330, or 337.

14. A conjugate comprising an anti-TfR antibody or antigen-binding fragment thereof coupled to:
(i) a therapeutic or diagnostic agent; or
(ii) a second antibody or an antigen-binding fragment thereof that binds to a brain target;
wherein the anti-TfR antibody or antigen-binding fragment thereof comprises
a heavy chain variable region comprising HCDRs HCDR1, HCDR2, and HCDR3 and a light chain variable region comprising LCDRs LCDR1, LCDR2, and LCDR3, wherein the HCDR1, HCDR2, HCDR3, LCDR1, LCDR2, and LCDR3 have the amino acid sequences of SEQ ID NOs: 279, 280, 281, 282, 283 and 284, respectively.

15. The conjugate of claim 14, wherein the anti-TfR antigen-binding fragment is a single-chain variable fragment (scFv) comprising an amino acid sequence having at least 90% sequence identity to the amino acid sequence of SEQ ID NO: 278.

16. The conjugate of claim 14, wherein the anti-TfR antibody or antigen-binding fragment thereof is covalently linked to a second antibody or antigen-binding fragment thereof, wherein the anti-TfR antibody or antigen-binding fragment thereof is covalently linked to the carboxy terminus of only one of the two heavy chains of the second antibody or antigen-binding fragment thereof via a linker, wherein the anti-TfR antibody or antigen-binding fragment thereof comprises an scFv comprising an amino acid sequence having at least 90% sequence identity to the amino acid sequence of SEQ ID NO: 278.
